(19) United States
(12) Patent Application Publication
(10) Pub. No.: US 2003/0003468 A1
(43) Pub. Date: Jan. 2, 2003

(54) Markers for disease susceptibility and targets for therapy

(75) Inventor: Mary K. Crow, New York, NY (US)

Correspondence Address: DARBY & DARBY P.C., 805 Third Avenue, New York, NY, 10022, US

(73) Assignee: Hospital for Special Surgery

(21) Appl. No.: 10/025,201

(22) Filed: Dec. 19, 2001

Related U.S. Application Data
(60) Provisional application No. 60/256,673, filed on Dec. 19, 2000.

(51) Int. Cl. C12Q 1/68

(52) U.S. Cl. 435/6

(57) ABSTRACT

The invention provides novel methods for determining susceptibility for, diagnosing, and/or treating complex diseases such as autoimmune diseases, Alzheimer's disease, and schizophrenia. The novel methods are based upon the finding that the positioning of long interspersed nuclear elements (LINEs) in the genome can play an important role in the induction and progression of complex diseases. Specifically, the distance and interaction between a LINE such as the L1 element and a susceptibility gene associated with a complex diseases can provide information about the risk for the complex disease, and also provide novel targets for therapy.

This application claims priority under 35 U.S.C. §119 of U.S. application Serial No. 60/256,673, filed Dec. 19, 2000.

### FIELD OF THE INVENTION

This invention pertains to complex diseases, including autoimmune diseases, methods to identify potential genes relevant to disease susceptibility, pathogenesis, and treatment; methods to determine an individual's susceptibility to be afflicted by these diseases, and methods to diagnose and treat these diseases.

### BACKGROUND OF THE INVENTION

Complex diseases are those with complex and poorly understood pathogenic mechanisms and that are not attributable to a mutation in a single gene. Among the complex diseases are the autoimmune diseases, as well as diseases such as Alzheimer disease and schizophrenia. Autoimmune diseases include the prototype, systemic lupus erythematosus (SLE), as well as the organ-targeted autoimmune diseases insulin-dependent diabetes mellitus (IDDM) and multiple sclerosis (MS). It has long been understood that genetic factors play an important role in susceptibility to these diseases. With the availability of molecular tools to define the sequence of the human (and model animal) genome, extensive investigations have attempted to define the genes that confer risk of developing these diseases (1-11). Knowing the identity of genes that place an individual at risk of developing a disease may permit identification of those at-risk individuals before disease onset or early in its course, allowing early institution of treatment. Identification of those genes also should lead to new understanding of disease mechanisms, through study of the role of their gene products, and other components of their molecular pathways, in normal human physiology and in patients. The gene or genes in question may be altered, resulting in abnormal function of its protein product, or it may be produced in too much or too little quantity. Most importantly, knowledge of disease susceptibility genes may lead to the development of new therapeutic approaches based on manipulation of the expression or activity of the particular gene product or of other gene products identified through understanding the activity of the disease gene.

The importance of disease genes has led to studies to identify complex disease susceptibility loci, through “genome screens”, often using analysis of microsatellites in human DNA at spaced locations throughout the genome (1). These markers of individual variability can be statistically analyzed to determine an association or linkage to certain phenotypic traits, such as diagnosis of a particular disease or expression of a laboratory or clinical manifestation of that disease. In this way, regions of the human genome that might identify a disease gene can be narrowed down and the specific gene eventually identified (12). In spite of studies attempting to define susceptibility genes in SLE, MS, IDDM, rheumatoid arthritis (RA), Crohn's disease, and schizophrenia, among others, very few of these genes have been identified. It would therefore be highly useful to develop a method to identify potential genes important in susceptibility to or pathogenesis of diseases. Knowing the identity of such disease genes would-provide an approach to predicting who will get disease, how the disease occurs, and most importantly, will advance development of new therapies for diseases.

As the prototype systemic autoimmune disease, SLE has served as an important model to consider the genetic and environmental factors that contribute to complex diseases. The idea that viruses may trigger SLE has always been a consideration, based on the systemic symptoms that are often typical of viral infection. Viruses have been sought, most successfully in animal models of SLE. Viral particles, particularly the gp70 envelope protein characteristic of some retroviruses, have been observed in the kidneys of lupus mice and humans (16). Recent work has documented full-length copies of several classes of endogenous retroviruses in human DNA, and transcription and translation of proteins encoded by these viral parasites have been documented. A role for endogenous retroviruses with long terminal repeat (LTR) sequences has been addressed in both IDDM and SLE. In IDDM, the data are conflicting and controversial (29-32). In SLE, efforts to document viral etiologic agents have had mixed successes. Virus-like inclusion bodies have been observed in the endothelial cells of kidneys from SLE patients, and RNA or DNA with virus-like sequences has been reported (33, 34). An endogenous retroviral sequence encoded on chromosome 1q, a chromosomal location enriched in potential disease susceptibility loci, has been studied (29, 35). Some patients with SLE were shown to produce autoantibodies specific for the product of that HERV sequence. However, no well-documented story has been developed that incorporates a role for viruses or virus-like DNA sequences in the genetic susceptibility factors that underlie complex diseases.

Another class of endogenous retrovirus-like elements, retrotransposons, also has gained increasing interest and has stimulated a novel model for induction of SLE and other complex diseases presented herein. Much of the “junk” DNA that is present in the genome derives from long interspersed nuclear elements (LINEs), comprising up to 20% of mammalian genomes (36). These DNA elements are fragments of a nucleotide sequence that has been distributed at many locations throughout the genome (37,38). Unlike retroviral sequences, LINEs do not have LTR regions at the 5′ and 3′ of the element sequence. When intact, meaning containing all parts of a consensus sequence from 5′ to 3′, they contain a 5′ regulatory region and two open reading frames (ORF) that can encode two proteins. The function of LINEs is to transcribe the two ORFs into mRNA, copy that RNA (or parts of it) into DNA, and insert that DNA back into the genome (39). It has been proposed that LINEs are an important engine of evolutionary change, perhaps mediating the shuffling of exons that generates biologic complexity (40-42).

The full-length human LINE-1 (L1) element is about 6000 bp in length (see, e.g., GenBank Accession No. U09116; SEQ ID NO 1. Other full-length LINE-1 sequences include GenBank Accession Nos. U93562; U93563; U93564; U93565; U93566; U93567; U93568; U93569; U93570; U93571; U93572; U93573; U93574; AF148856; and AF149422. A nearly 900 bp 5′ untranslated regulatory region is followed by a 984 bp ORF that encodes a 40 kD protein (p40; SEQ ID NO:2) with an NH₂-terminal leucine zipper-like domain, possibly mediating protein interactions (44). For both human and murine ORF 1, the 5′ end is highly divergent (36). In common are enrichment in CpG sequences and an absence of TATA boxes (52). Several studies have investigated the 5′ regulatory motifs that are essential for effective L1 gene transcription. An important motif is found within the 5′ 30 bp of the L1 consensus sequence (53). The motif includes a G-rich sequence that binds the YY1 protein, a ubiquitous DNA binding protein that can act either as an activator or repressor. In the case of human L1, alteration of the YY1 binding site substantially reduced transcriptional activity. Of interest, additional sequences upstream of the 5′ consensus sequence also appeared to affect L1 transcription. Those sequences have neither been defined nor functionally characterized. Two additional important regulatory elements have recently been defined. Binding sites for proteins of the SOX family, located between nucleotides 472 and 477 and between nucleotides 572 and 577, have been studied (85). The male-restricted Y chromosome encoded SRY protein, the prototype of the SOX family of transcriptional regulatory proteins, binds to these two elements and inhibits LINE transcription, while other members of the SOX family bind to the same elements and increase transcription. These findings suggest that LINE transcription may be differentially regulated in males and females.

The nucleic acid binding properties of ORF1 p40 have been studied, and the protein has been shown to preferentially bind to single-stranded RNA (45). Interestingly, p40 has relative specificity for sense strand ORF2 RNA coding regions. While the function of p40 is not known, and it bears little sequence homology to known proteins, the basic COOH-domain of the protein has been mutated and shown to be essential for retrotransposition of the element in an in vitro cell culture assay. A short intervening sequence separates ORF1 from an approximately 3800 bp ORF2 coding sequence, encoding the protein represented by SEQ ID NO:3. The full-length L1 transcript, including ORF1, intron, and ORF2, is localized in cytoplasmic ribonucleoproteins (RNPs) particles with p40, and ORF2 is ultimately translated into a protein with both typical reverse transcriptase and endonuclease domains (44,46-48). As is true for ORF1 p40, both endonuclease and reverse transcriptase domains of ORF2 protein are essential for retrotransposition in vitro (49-51).

### SUMMARY OF THE INVENTION

The present invention is based on the surprising discovery that the proximity of a LINE element such as L1 to a region of the genome associated with a diagnosis of a complex disease or susceptibility to a complex disease can indicate the identity of a gene or genes involved in the pathogenesis of that disease. Moreover, individual variability in the presence or nucleotide sequence of a LINE element in proximity to or within an intronic region of one or more genes associated with or involved in the development of a disease can be an indicator of an individual's susceptibility to the disease. Additionally, the detection of DNA, mRNA or protein encoded by a LINE element in the cells or body fluid of a patient with a complex disease can be used to diagnose or measure the activity of that disease, and the detection of antibodies reactive with DNA, RNA, or proteins encoded by a LINE element can be used to diagnose or measure the activity of that disease. Finally, it may be useful to inhibit the expression or activity of LINE nucleotide and protein products as a therapeutic approach in patients with complex disease. In particular, the method is applicable for complex diseases such as, e.g., autoimmune diseases, Alzheimer's disease, and schizophrenia.

Thus, the present invention provides for a method of identifying genes and gene products that are involved in susceptibility to and pathogenesis of a complex disease. Information regarding disease susceptibility loci available in the literature can be used to direct computer-based searches to a region of the genome neighboring a disease-associated marker. Comparison of the sequence of the 5′ regulatory region of a consensus L1 sequence to that genome region is used to localize full-length and full-length high fidelity L1 sequences to the intronic region of genes or predicted genes or to the 5′ or 3′ regulatory region of genes or predicted genes. Those genes containing a full-length L1 element in their intronic region or containing a full-length L1 element with high sequence fidelity to the consensus sequence in their 5′ or 3′ regulatory region are identified as potential disease genes. Alternatively, a catalogue of such genes can be generated and used as a database for study of potential disease genes relevant to various and numerous diseases. The present invention also provides for a method of identifying an individual at risk for or suffering from a complex disease, which method comprises investigating the individual's DNA in the intronic regions of genes containing full-length L1 elements or in the 5′ or 3′ regulatory regions of genes containing a full-length high fidelity consensus L1 sequence. For a given disease, a preferred method would involve directing the DNA study to those areas of the genome associated with a diagnosis of or susceptibility to that complex disease. The DNA sample can suitably be prepared from a tissue sample taken from the individual. By any method commonly used to obtain the sequence of or detect the presence of a genomic DNA segment, the region of DNA including the 5′ regulatory region of the L1 sequence and the adjacent genomic sequence are sequenced or identified. In one embodiment, the high-fidelity L1 sequence is present in the intronic region or 5′ or 3′ regulatory region of a gene in the DNA of the test individual, but not in the DNA of control individuals. In another embodiment, the sequence of the 5′ regulatory region of the L1 element in the DNA of the test individual is of higher fidelity to the L1 consensus sequence than in the DNA of control individuals. In a third embodiment, nucleotides in the 5′ regulatory region of the L1 sequence that have an important role in controlling L1 transcription will be present in the test individual but not in control individuals. Typically, the most 5′ approximately 30 nucleotides from the sequence of SEQ ID NO:1 will be identified in the context of the adjacent genomic sequence to determine the presence of a given L1 element. Alternatively, the sequence of the most 5′ approximately 884 nucleotides of SEQ ID NO:1, or another consensus L1 sequence, will be compared with the corresponding L1 sequence in the DNA of the test individual and control individuals. In one embodiment, a full-length L1 element in the intronic region of a gene has sequence identity to a consensus sequence, as that of SEQ ID NO:1, ranging from 75-100% and includes the full nucleotide sequence, or is only absent up to the first 20 nucleotides of the consensus sequence. In another embodiment, a high-fidelity L1 sequence in the intronic region or in the 5′ or 3′ regulatory region of a gene can be at least about 97% similar to the sequence of nucleotides 1-884 of SEQ ID NO:1, or, alternatively, identical to residues 1-884 of SEQ ID NO:1. In another embodiment, the DNA of the test individual will have a nucleotide alteration in a putative regulatory region contained within residues 1-884 of SEQ ID NO:1. The method is applicable for a variety of complex diseases, including systemic lupus erythematosus (SLE), multiple sclerosis (MS), insulin-dependent diabetes mellitus (IDDM), rheumatoid arthritis (RA), phemphigus, psoriasis, autoimmune thyroid disease, scleroderma, mixed connective tissue disease, polymyositis, dermatomyositis, Sjögren's syndrome, pemphigoid, vitiligo, primary biliary cirrhosis, chronic active hepatitis, Crohn's disease, ulcerative colitis, pernicious anemia, schizophrenia, and Alzheimer disease.

In addition, the invention provides for a method of identifying an individual susceptible to or at risk for or with activity of a complex disease by detecting the level of L1 DNA, mRNA or a protein encoded by an L1 element in the tissue, cell, or body fluid sample taken from the individual, wherein the individual is susceptible to or at risk for or currently affected by the complex disease if the level is higher than the level in a control sample. The tissue, cell, or body fluid sample can be taken from blood, serum, saliva, urine, tears, sweat, synovial fluid, cerebrospinal fluid, or from a solid tissue. The L1 DNA is preferably detected in a body fluid and is at least 80% identical to SEQ ID NO:1. L1 mRNA is preferably complementary to SEQ ID NO:1, or to a sequence preferably at least 95%, homologous to SEQ ID NO:1 and extending to within 20 nucleotides, preferably 10 nucleotides, of the 5′ end of a consensus sequence identical to SEQ ID NO:1. A protein encoded by an L1 element can be encoded by ORF1 or ORF2 of a sequence preferably at least 95% homologous to SEQ ID NO:1. The L1mRNA may be part of a ribonucleoprotein, and the protein encoded by an L1 element can be either ORF1 and ORF2, or a combination of both.

Furthermore, the invention provides for a method to identify an individual susceptible to or at risk for or with activity of a complex disease by detecting antibodies to DNA or RNA with at least 80% sequence identity to SEQ ID NO:1 or by detecting antibodies to the protein products of an L1 element. The antibodies for the L1 protein product can bind to the protein encoded by either ORF1 and ORF2, or a combination of both, and they may detect DNA, RNA, or ORF1 or ORF2 proteins that are part of a ribonucleoprotein particle.

Furthermore, the invention provides for a method of treating or preventing a complex disease, comprising administering a therapeutically effective amount of an agent such as an L1 antisense oligonucleotide, an agent that inhibits the transcription of L1 mRNA, an antibody directed against L1 mRNA, and/or an antibody or other molecule directed against a protein encoded by an L1 element.

In one aspect, the present invention provides a method of identifying a gene involved in a complex disease comprising the steps of identifying a region of the genome neighboring a disease-associated marker; comparing the sequence of the 5′ regulatory region of a consensus L1 sequence to the intronic region of genes or predicted genes or to the 5′ or 3′ regulatory region of genes or predicted genes; and identifying genes containing a full-length L1 element in their intronic region or containing a full-length L1 element with high sequence fidelity to the L1 consensus sequence in their 5′ or 3′ regulatory region, wherein said genes identified in step (iii) are involved in a complex disease.

In another aspect, the present invention provides a method of identifying an individual at risk for or suffering from a complex disease comprising the steps of providing a sample from the individual; identifying intronic regions of genes containing full-length L1 elements or in 5′ or 3′ regulatory regions of genes containing a full-length high fidelity consensus L1 sequence of the individual's DNA from the sample; and comparing said intronic regions of genes or said 5′ or 3′ regulatory regions of step (ii) with a control sample of DNA taken from an individual not susceptible to or at risk for or currently suffering from a complex disease wherein said genes identified in step (ii) are involved in a complex disease.

In yet another aspect, the present invention provides a method of identifying an individual at risk for or suffering from a complex disease comprising the steps of providing a sample from the individual suffering from a complex disease; detecting the amount of L1 DNA, mRNA or a protein encoded by an L1 element in the sample; and comparing the amount of step (ii) with an amount of L1 DNA, mRNA or a protein obtained from an individual not susceptible to or at risk for or suffering from a complex disease, wherein if the amount detected in the sample obtained from the individual is greater than the amount of the control, the individual is at risk for or suffering from a complex disease.

In a further aspect, the present invention provides A method for identifying an individual at risk for or suffering from a complex disease comprising the steps of providing a sample obtained from the individual; detecting antibodies directed against ribonucleo-protein particles having L1 mRNA complements in the sample wherein the individual is at risk for or is suffering from a complex disease if the antibodies are present in the sample.

In yet a further aspect, the present invention provides a method of identifying an individual at risk for or suffering from a complex disease comprising the steps of providing a sample obtained from the individual; analyzing the sample for the presence of auto antibodies directed against L1 DNA, nRNA or protein products wherein the individual is at risk for or suffering from a complex disease if the antibodies are present in the sample.

These and other aspects of the present invention will be apparent to those of ordinary skill in the art in light of the present specification, claims and drawings.

### BRIEF DESCRIPTION OF THE DRAWINGS

FIG. 1. This figure shows the DNA sequence of the primer pairs for PCR amplification of an L1 element on human chromosome 1q. Nucleotides 15721 to 14892 (SEQ ID NO:4) of BAC clone AL162431 were analyzed to identify nucleotide sequences of primary 5′ and 3′ PCR primers (solid lines) and secondary nested 5′ and 3′ primers (dotted lines), shown bracketed, for amplification of a chromosomal segment that is specific to the chromosome 1q location 5′ to the L1 sequence, along with the adjacent 5′ regulatory region of the L1 element. 5′ primary and secondary nested primers are identical to the indicated sequences. 3′ primary and secondary nested primers are complementary to the indicated sequences.

FIG. 2. This figure shows that SLE susceptibility loci with high LOD scores are associated with proximity to full-length, high fidelity L1 elements or full-length L1 elements within the coding sequences of genes on chromosome 1q. The location of L1 elements is indicated with a bar, and a free-hand drawing replicating the data from microsatellite analysis of SLE susceptibility loci, derived from reference 4, is superimposed on the figure representing chromosome 1q.

FIG. 3. This figure shows that SLE susceptibility loci with high LOD socres are associated with proximity to full-length, high fidelity L1 elements or full-length L1 elements within the coding sequences of genes on chromosome 16. The location of L1 elements is indicated with a bar, and a free-hand drawing replicating the data from microsatellite analysis of SLE susceptibility loci, derived from reference 4, is superimposed on the figure representing chromosome 16.

FIG. 4. This figure shows that 3 genes on chromosome 21 contain full-length L1 elements in their coding regions. The location of L1 elements is indicated with a bar, and a free-hand drawing replicating the data from microsatellite analysis of SLE susceptibility loci, derived from reference 4, is superimposed on the figure representing chromosome 21.

FIG. 5. This figure shows expression of L1 ORF1 mRNA in NTERA-D1 cells. NTERA and HeLa cell line cells were cultured for 48 h with medium or with 5-azacytidine (5-Aza) at 0.5, 1, or 5 micromolar. Total RNA was isolated, reverse transcribed, and amplified in a competitive PCR assay for L1 ORF1 mRNA. 1 ml of cDNA, 1 ml of each of three concentrations of an ORF1-containing MIMIC (20, 10, and 5 attomoles/ml; generated using the Clontech MIMIC construction kit), 0.5 ml of ORF1-specific primers, and 22.5 ml of PCR super mix (Life Technologies, Gaithersburg, Md.) were combined and PCR was carried out by denaturing at 94° C. for 45 sec, annealing at 55° C. for 45 sec, and with extension at 72° C. for 1 min. The dilution of mimic which produced a band of equal intensity to that of target cDNA was determined. The 3 mimic concentrations are shown sequentially across the gel in triplicate FIGS. 6(A, B and C). This figure shows Western blot analysis of L1 ORF1 p40 protein. (A) Total cellular extracts were prepared from NTERA-D1 and HeLa cell line cells. Extracts were enriched in RNP particles by centrifugation at 160,000 g for 2.5 h. Proteins (50 microg/lane) were resolved on a 10% gel, transferred to an Immobilon-P membrane and immunoblotted with 1:1000 rabbit anti-p40 antibody. (B) T and non-T cells were fractionated from peripheral blood isolated from an SLE patient. The RNP fraction was isolated, 10 mg protein loaded per lane, and resolved proteins immunoblotted with rabbit anti-p40 antibody. T and non-T cells were fractionated from peripheral blood samples from three SLE patients and one healthy control individual. (C) Cell protein extracts were prepared, 50 mg protein loaded per lane and electrophoresed, and the resolved proteins immunoblotted with rabbit anti-p40antibody. The bands corresponding to the 40 kD ORF1 protein and a non-specific band at 95 kD are indicated by arrows.

FIG. 7. Western blot analysis of sera from SLE patients, healthy controls, a lupus mouse, and a control mouse. Recombinant human L1 ORF1 p40 protein was electrophoresed, transferred to a nitrocellulose filter, and then overlayed with sera . Antibody reactive with the p40 L1 protein is detected in sera from the MRL/lpr mouse, several SLE sera, and faintly in one control serum sample.

### DETAILED DESCRIPTION OF THE INVENTION

All patent applications, patents and literature references cited herein are hereby incorporated by reference in their entirety.

The present invention is directed to the use of endogenous DNA elements with sequence properties of viruses, but that do not meet the definition of true viruses, that are involved in the development of “complex” diseases such as, but not limited to systemic autoimmune diseases, organ-specific autoimmune diseases, SLE, Alzheimer disease, and schizophrenia. In a preferred embodiment of the present invention, the endogenous DNA elements are LINE retrotransposons.

The present invention further provides a method for evaluating L1 elements as markers of disease genes, susceptibility factors, pathogenic triggers or mediators of complex diseases, including systemic and organ targeted autoimmune diseases. Additionally, the present invention discloses the use of L1 elements and their products as therapeutic targets in systemic and organ targeted autoimmune diseases and other complex diseases.

### Definitions

As used herein, “complex diseases” are defined as multigenic diseases characterized by complex and poorly understood pathogenic mechanisms. Non-limiting examples of complex diseases include SLE, MS, IDDM, RA, psoriasis, autoimmune thyroid disease, scleroderma, mixed connective tissue disease, polymyositis, dermatomyositis, Sjögren's syndrome, pemphigoid, pemphigus vulgaris, pemphigus foliaceus, vitiligo, primary biliary cirrhosis, chronic active hepatitis, Crohn's disease, ulcerative colitis, pernicious anemia, schizophrenia, and Alzheimer disease. An individual “at risk for”, “predisposed to”, or “susceptible to” a disease or condition means that the risk for the individual to contract or develop the disease or condition is higher than in the average population.

A “high fidelity” L1 element means a sequence that shows at least about 97%, about 98%, about 99%, or up to about 100% sequence homology to a consensus L1 element or sequence, preferably a human consensus L1 element. A “moderate fidelity” L1 element means a sequence that shows at least about 75%, about 80%, about 85%, about 90%, or about 95% sequence homology to a consensus L1 sequence.

A “consensus sequence” is the sequence that reflects the most common choice of base or amino acid at each position of a series of related DNA, RNA or protein sequences. Areas of particularly good agreement frequently, although not necessarily, represent conserved functional domains. SEQ ID NO:1 is denoted as an L1 consensus sequence, or consensus element, herein.

A “consensus L1 element” can comprise at least about 30, about 200, about 400, about 600, about 800, or about 1000 nucleotide residues of an L1 element, and is preferably derived from the 5′ regulatory region. A preferred L1 element consensus sequence is a sequence derived from or corresponding to GenBank Accession No. U09116 (SEQ ID NO:1). In one embodiment, the L1 consensus sequence comprises, at least, about 30, about 200, about 400, about 600, about 800, or about 1000 nucleotides of the first (5′) 1000 or 2000 nucleotides of SEQ ID NO:1. In a preferred embodiment, the L1 consensus sequence comprises nucleotides 1-884 of SEQ ID NO:1. In another preferred embodiment, the L1 consensus sequence comprises the full-length 5′ regulatory region and approximately 5′ one third of the 5′ ORF1 sequence.

A “susceptibility locus” for a particular disease is a sequence or gene locus implicated in the initiation or progression of the disease. The susceptibility locus can be, for example, a gene or a microsatellite repeat, as identified by a microsatellite marker, or can be identified by a defined single nucleotide polymorphism. The specific genes associated with most susceptibility loci have not been identified, although many putative disease genes have been investigated. Examples of complex disease/proposed susceptibility gene locus pairs include: Graves disease/thyroid stimulating hormone receptor; primary biliary cirrhosis/S P100; pemphigus vulgaris or foliaceus/desmoglein 1 or 3; vitiligo/tyrosinase related protein 2; SLE/FcgRIIb; Alzheimer disease/APP; schizophrenia/DISC1 and CHRNA7; IDDM/insulin. Various disease susceptibility markers for SLE are also provided in Table 1 and for schizophrenia in Table 2.

Generally, susceptibility genes implicated in specific diseases and their loci can be found in scientific publications, but may also be determined experimentally. For purposes of the present invention, the “locus” of a susceptibility gene refers to the most 5′ nucleotide in the coding sequence for the susceptibility gene. As the sequencing of the human genome is still in progress, precise locations and DNA sequences of genes and disease loci remain subject to revision pending completion of the full genome analysis in multiple individuals.

A “microsatellite repeat” or “microsatellite” can also be an indicator to “susceptibility” of certain complex diseases, such as Crohn's disease, schizophrenia, and SLE as described herein. The term “microsatellite repeat” refers to a short sequence of repeating nucleotides within a nucleic acid. Typically, a microsatellite repeat comprises a repeating sequence of two (i.e., a dinucleotide repeat), three (i.e., a trinucleotide repeat), four (i.e., a tetranucleotide repeat) or five (i.e., a pentanucleotide repeat) nucleotides. Microsatellites of the invention therefore have the general formula (N₁, N₂, . . . Nᵢ)ₙ, wherein N represents a nucleic acid residue (e.g., adenine, thymine, cytosine or guanine), “i” represents the number of the last nucleotide in the microsatellite, and “n” represents the number of times the motif is repeated in the microsatellite locus. In one embodiment the number of nucleotides in a microsatellite motif “i” is about six, preferably between two and five, and more preferably two, three or four. The total number of repeats “n” in a microsatellite repeat may be, e.g., from one to about 60, preferably from 4 to 40, and more preferably from 10 to 30 when i=2; is preferably between about 4-25, and more preferably between about 6-22 when i=3; and is preferably between about 4-15, and more preferably between about 5-10 when i=4.

A “control”, “control value” or “reference value” in an assay is a value used to detect an alteration in, e.g., transcriptional activity of a gene, levels of a protein or mRNA detected in a sample taken from a patient or measured in a reconstituted system, or any other assays described herein. For instance, the presence or expression of an L1 element can be tested or verified by measuring the levels of mRNA or ORF protein in a tissue sample from an individual at risk and compare the results to a control. In addition, modulation, i.e., up- or down-regulation, of the transcriptional activity of an L1 element or the inhibitory/stimulatory effect of an agent on modulation can be evaluated by comparing the measured value of transcriptional activity to that of a control value. The control or reference value may be, e.g., a predetermined reference value, or may be determined experimentally. For example, in such an assay, a control or reference may be, e.g., the transcriptional activity of a gene in the absence of an agent (to comparison with transcriptional activity in the presence of the agent); or any other suitable control or reference. In a diagnostic assay, a reference or control value may be obtained by comparing e.g., a nucleotide sequence, or a nucleotide or protein level measured, in a sample taken from a patient predisposed to or suspected of suffering from, a disease, to a corresponding sequence or measured value of a sample taken from a healthy, or “control” individual.

### General

A “sample” refers to a biological material which can be tested for the presence of L1 elements. Such samples can be obtained from subjects, such as humans and non-human animals, and include tissue, especially glands, biopsies, blood and blood products; plural effusions; cerebrospinal fluid (CSF); ascites fluid; and cell culture.

The term “ability to elicit a response” includes the ability of a ligand to agonize or antagonize activity.

The term “transformed cell” refers to a modified host cell that expresses a functional protein expressed from a vector encoding the protein of interest. Any cell can be used, but preferred cells are mammalian cells.

A “test compound” is any molecule, that can be tested for its ability to modulate L1 expression and/or activity.

### Molecular Biology

In accordance with the present invention there may be employed conventional molecular biology, microbiology, and recombinant DNA techniques within the skill of the art. Such techniques are explained fully in the literature. See, e.g., Sambrook, Fritsch & Maniatis, Molecular Cloning: A Laboratory Manual, Second Edition (1989) Cold Spring Harbor Laboratory Press, Cold Spring Harbor, N.Y. (herein “Sambrook et al., 1989”); DNA Cloning: A Practical Approach, Volumes I and II (D. N. Glover ed. 1985); Oligonucleotide Synthesis (M. J. Gait ed. 1984); Nucleic Acid Hybridization (B. D. Hames & S. J. Higgins eds. (1985)); Transcription And Translation (B. D. Hames & S. J. Higgins, eds. (1984)); Animal Cell Culture (R. I. Freshney, ed. (1986)); Immobilized Cells And Enzymes (IRL Press, (1986)); B.Perbal, A Practical Guide To Molecular Cloning (1984); F. M. Ausubel et al. (eds.), Current Protocols in Molecular Biology, John Wiley & Sons, Inc. (1994).

A “nucleic acid molecule” refers to the phosphate ester polymeric form of ribonucleosides (adenosine, guanosine, uridine or cytidine; “RNA molecules”) or deoxyribonucleosides (deoxyadenosine, deoxyguanosine, deoxythymidine, or deoxycytidine; “DNA molecules”), or any phosphoester analogs thereof, such as phosphorothioates and thioesters, in either single stranded form, or a double-stranded helix. Double stranded DNA-DNA, DNA-RNA and RNA-RNA helices are possible. The term nucleic acid molecule, and in particular DNA or RNA molecule, refers only to the primary and secondary structure of the molecule, and does not limit it to any particular tertiary forms. Thus, this term includes double-stranded DNA found, inter alia, in linear (e.g., restriction fragments) or circular DNA molecules, plasmids, and chromosomes. In discussing the structure of particular double-stranded DNA molecules, sequences may be described herein according to the normal convention of giving only the sequence in the 5′ to 3′ direction along the nontranscribed strand of DNA (i.e., the strand having a sequence homologous to the mRNA). A “recombinant DNA molecule” is a DNA molecule that has undergone a molecular biological manipulation.

A “polynucleotide”, “nucleotide sequence”, or “oligonucleotide” is a series of nucleotide bases (also called “nucleotides”) in DNA and RNA, and means any chain of two or more nucleotides. A nucleotide sequence typically carries genetic information, including the information used by cellular machinery to make proteins and enzymes. These terms include double or single stranded genomic and cDNA, RNA, any synthetic and genetically manipulated polynucleotide, and both sense and anti-sense polynucleotide (although only sense stands are being represented herein). This includes single- and double-stranded molecules, i.e., DNA-DNA, DNA-RNA and RNA-RNA hybrids, as well as “protein nucleic acids” (PNA) formed by conjugating bases to an amino acid backbone. This also includes nucleic acids containing modified bases, for example thio-uracil, thio-guanine and fluoro-uracil.

An oligonucleotide comprising at least 10, preferably at least 15, and more preferably at least 20 nucleotides, preferably no more than 100 nucleotides, can be hybridizable to a genomic DNA molecule, a cDNA molecule, or an mRNA molecule encoding a gene, mRNA, cDNA, or other nucleic acid of interest. Oligonucleotides can be labeled, e.g., with ³²P-nucleotides or nucleotides to which a label, such as biotin, has been covalently conjugated. In one embodiment, a labeled oligonucleotide can be used as a probe to detect the presence of a nucleic acid. In another embodiment, oligonucleotides (one or both of which may be labeled) can be used as PCR primers, either for cloning full length or a fragment of L1, or to detect the presence of nucleic acids encoding L1. In a further embodiment, an oligonucleotide of the invention can form a triple helix with a L1 DNA molecule. Generally, oligonucleotides are prepared synthetically, preferably on a nucleic acid synthesizer. Accordingly, oligonucleotides can be prepared with non-naturally occurring phosphoester analog bonds, such as thioester bonds, etc.

The present invention also provides antisense nucleic acids (including ribozymes), which may be used to inhibit expression of L1 elements of the invention. An “antisense nucleic acid” is a single stranded nucleic acid molecule which, on hybridizing under cytoplasmic conditions with complementary bases in an RNA or DNA molecule, inhibits the latter's role. If the RNA is a messenger RNA transcript, the antisense nucleic acid is a countertranscript or mRNA-interfering complementary nucleic acid. As presently used, “antisense” broadly includes RNA-RNA interactions, RNA-DNA interactions, ribozymes and RNase-H mediated arrest. Antisense nucleic acid molecules can be encoded by a recombinant gene for expression in a cell (e.g., U.S. Pat. Nos. 5,814,500; 5,811,234), or alternatively they can be prepared synthetically (e.g., U.S. Pat. No. 5,780,607).

As used herein, “sequence-specific oligonucleotides” refers to related sets of oligonucleotides that can be used to detect allelic variations or mutations in the L1 element.

“Amplification” of DNA as used herein denotes the use of polymerase chain reaction (PCR) to increase the concentration of a particular DNA sequence within a mixture of DNA sequences. For a description of PCR see Saiki et al., Science, 239:487, 1988.

Specific non-limiting examples of synthetic oligonucleotides envisioned for this invention include oligonucleotides that contain phosphorothioates, phosphotriesters, methyl phosphonates, short chain alkyl, or cycloalkyl intersugar linkages or short chain heteroatomic or heterocyclic intersugar linkages. Most preferred are those with CH₂—NH—O—CH₂, CH₂—N(CH₃)—O—CH₂, CH₂—O—N(CH₃)—CH₂, CH₂—N(CH₃)—N(CH₃)—CH₂ and O—N(CH₃)—CH₂—CH₂ backbones (where phosphodiester is O—PO₂—O—CH₂). U.S. Pat. No. 5,677,437 describes heteroaromatic olignucleoside linkages. Nitrogen linkers or groups containing nitrogen can also be used to prepare oligonucleotide mimics (U.S. Pat. Nos. 5,792,844 and No. 5,783,682). U.S. Pat. No. 5,637,684 describes phosphoramidate and phosphorothioamidate oligomeric compounds. Also envisioned are oligonucleotides having morpholino backbone structures (U.S. Pat. No. 5,034,506). In other embodiments, such as the peptide-nucleic acid (PNA) backbone, the phosphodiester backbone of the oligonucleotide may be replaced with a polyamide backbone, the bases being bound directly or indirectly to the aza nitrogen atoms of the polyamide backbone (82). Other synthetic oligonucleotides may contain substituted sugar moieties comprising one of the following at the 2′ position: OH, SH, SCH3, F, OCN, O(CH₂)ₙNH₂ or O(CH₂)ₙCH₃ where n is from 1 to about 10; C₁ to C₁₀ lower alkyl, substituted lower alkyl, alkaryl or aralkyl; Cl; Br; CN; CF3; OCF₃; O-; S-, or N-alkyl; O-, S-, or N-alkenyl; SOCH₃; SO₂CH₃; ONO₂; NO₂; N₃; NH₂; heterocycloalkyl; heterocycloalkaryl; aminoalkylamino; polyalkylamino; substitued silyl; a fluorescein moiety; an RNA cleaving group; a reporter group; an intercalator; a group for improving the pharmacokinetic properties of an oligonucleotide; or a group for improving the pharmacodynamic properties of an oligonucleotide, and other substituents having similar properties. Oligonucleotides may also have sugar mimetics such as cyclobutyls or other carbocyclics in place of the pentofuranosyl group. Nucleotide units having nucleosides other than adenosine, cytidine, guanosine, thymidine and uridine, such as inosine, may be used in an oligonucleotide molecule.

The polynucleotides herein may be flanked by natural regulatory (expression control) sequences, or may be associated with heterologous sequences, including promoters, internal ribosome entry sites (IRES) and other ribosome binding site sequences, enhancers, response elements, suppressors, signal sequences, polyadenylation sequences, introns, 5′- and 3′-non-coding regions, and the like. The nucleic acids may also be modified by many means known in the art. Non-limiting examples of such modifications include methylation, “caps”, substitution of one or more of the naturally occurring nucleotides with an analog, and internucleotide modifications such as, for example, those with uncharged linkages (e.g., methyl phosphonates, phosphotriesters, phosphoroamidates, carbamates, etc.) and with charged linkages (e.g., phosphorothioates, phosphorodithioates, etc.). Polynucleotides may contain one or more additional covalently linked moieties, such as, for example, proteins (e.g., nucleases, toxins, antibodies, signal peptides, poly-L-lysine, etc.), intercalators (e.g., acridine, psoralen, etc.), chelators (e.g., metals, radioactive metals, iron, oxidative metals, etc.), and alkylators. The polynucleotides may be derivatized by formation of a methyl or ethyl phosphotriester or an alkyl phosphoramidate linkage. Furthermore, the polynucleotides herein may also be modified with a label capable of providing a detectable signal, either directly or indirectly. Exemplary labels include radioisotopes, fluorescent molecules, biotin, and the like.

A “coding sequence” or a sequence “encoding” an expression product, such as a RNA, polypeptide, protein, or enzyme, is a nucleotide sequence that, when expressed, results in the production of that RNA, polypeptide, protein, or enzyme, i.e., the nucleotide sequence encodes an amino acid sequence for that polypeptide, protein or enzyme. A coding sequence for a protein may include a start codon (usually ATG) and a stop codon.

The term “gene”, also called a “structural gene” means a DNA sequence that codes for or corresponds to a particular sequence of amino acids which comprise all or part of one or more proteins or enzymes, and may or may not include introns and regulatory DNA sequences, such as promoter sequences, 5′-untranslated region, or 3′-untranslated region which affect for example the conditions under which the gene is expressed. Some genes, which are not structural genes, may be transcribed from DNA to RNA, but are not translated into an amino acid sequence. Other genes may function as regulators of structural genes or as regulators of DNA transcription.

A “promoter sequence” is a DNA regulatory region capable of binding RNA polymerase in a cell and initiating transcription of a downstream (3′ direction) coding sequence. For purposes of defining the present invention, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation site (conveniently defined for example, by mapping with nuclease S1), as well as protein binding domains (consensus sequences) responsible for the binding of RNA polymerase.

An “intron” is a non-coding sequence of DNA within a gene, that is transcribed into hnRNA but is then cut out by RNA splicing in the nucleus, leaving a mature mRNA that is then translated in the cytoplasm. Introns are poorly conserved and of variable length, but the regions at the ends are self complementary, allowing a hairpin structure to form naturally in the hnRNA, this is the cue for removal by RNA splicing. Introns are thought to play an important role in allowing rapid evolution of proteins by exon shuffling. Genes may contain as many as 80 introns.

An “exon” is a sequences of the primary RNA transcript (or the DNA that encodes them) that exits the nucleus as part of a messenger RNA molecule. In the primary transcript neighboring exons are separated by introns.

A coding sequence is “under the control of” or “operatively associated with” transcriptional and translational control sequences in a cell when RNA polymerase transcribes the coding sequence into mRNA, which is then trans-RNA spliced (if it contains introns) and translated, in the case of mRNA, into the protein encoded by the coding sequence.

The terms “express” and “expression” mean allowing or causing the information in a gene or DNA sequence to become manifest, for example producing a protein by activating the cellular functions involved in transcription and translation of a corresponding gene or DNA sequence. A DNA sequence is expressed in or by a cell to form an “expression product” such as a protein. The expression product itself, e.g. the resulting protein, may also be said to be “expressed” by the cell. An expression product can be characterized as intracellular, extracellular or secreted. The term “intracellular” means something that is inside a cell. The term “extracellular” means something that is outside a cell. A substance is “secreted” by a cell if it appears in significant measure outside the cell, from somewhere on or inside the cell.

The terms “vector”, “cloning vector” and “expression vector” mean the vehicle by which a DNA or RNA sequence (e.g. a foreign gene) can be introduced into a host cell, so as to transform the host and promote expression (e.g. transcription and translation) of the introduced sequence. Vectors include plasmids, phages, viruses, etc.; they are discussed in greater detail below.

Vectors typically comprise the DNA of a transmissible agent, into which foreign DNA is inserted. A common way to insert one segment of DNA into another segment of DNA involves the use of enzymes called restriction enzymes that cleave DNA at specific sites (specific groups of nucleotides) called restriction sites. A “cassette” refers to a DNA coding sequence or segment of DNA that codes for an expression product that can be inserted into a vector at defined restriction sites. The cassette restriction sites are designed to ensure insertion of the cassette in the proper reading frame. Generally, foreign DNA is inserted at one or more restriction sites of the vector DNA, and then is carried by the vector into a host cell along with the transmissible vector DNA. A segment or sequence of DNA having inserted or added DNA, such as an expression vector, can also be called a “DNA construct.” A common type of vector is a “plasmid”, which generally is a self-contained molecule of double-stranded DNA, usually of bacterial origin, that can readily accept additional (foreign) DNA and which can readily introduced into a suitable host cell. A plasmid vector often contains coding DNA and promoter DNA and has one or more restriction sites suitable for inserting foreign DNA. Coding DNA is a DNA sequence that encodes a particular amino acid sequence for a particular protein or enzyme. Promoter DNA is a DNA sequence which initiates, regulates, or otherwise mediates or controls the expression of the coding DNA. Promoter DNA and coding DNA may be from the same gene or from different genes, and may be from the same or different organisms. A large number of vectors, including plasmid and fungal vectors, have been described for replication and/or expression in a variety of eukaryotic and prokaryotic hosts. Non-limiting examples include pKK plasmids (Clonetech), pUC plasmids, pET plasmids (Novagen, Inc., Madison, Wis.), pRSET or pREP plasmids (Invitrogen, San Diego, Calif.), or pMAL plasmids (New England Biolabs, Beverly, Mass.), and many appropriate host cells, using methods disclosed or cited herein or otherwise known to those skilled in the relevant art. Recombinant cloning vectors will often include one or more replication systems for cloning or expression, one or more markers for selection in the host, e.g. antibiotic resistance, and one or more expression cassettes.

The terms “mutant” and “mutation” mean any detectable change in genetic material, e.g. DNA, or any process, mechanism, or result of such a change. This includes gene mutations, in which the structure (e.g. DNA sequence) of a gene is altered, any gene or DNA arising from any mutation process, and any expression product (e.g. protein or enzyme) expressed by a modified gene or DNA sequence. The term “variant” may also be used to indicate a modified or altered gene, DNA sequence, enzyme, cell, etc., i.e., any kind of mutant.

The term “homologous”, in all its grammatical forms and spelling variations, refers to the relationship between two proteins that possess a “common evolutionary origin”, including proteins from superfamilies (e.g., the immunoglobulin superfamily) in the same species of organism, as well as homologous proteins from different species of organism (for example, myosin light chain polypeptide, etc.; see, Reeck et al., Cell 1987;50:667). Such proteins (and their encoding nucleic acids) have sequence homology, as reflected by their sequence similarity, whether in terms of percent identity or by the presence of specific residues or motifs and conserved positions.

The term “heterologous” refers to a combination of elements not naturally occurring. For example, heterologous DNA refers to DNA not naturally located in the cell, or in a chromosomal site of the cell. Preferably, the heterologous DNA includes a gene foreign to the cell. A heterologous expression regulatory element is such an element operatively associated with a different gene than the one it is operatively associated with in nature. In the context of the present invention, an L1 gene is heterologous to the vector DNA in which it is inserted for cloning or expression, and it is heterologous to a host cell containing such a vector, in which it is expressed, e.g., a HUVEC cell.

The term “sequence similarity”, in all its grammatical forms, refers to the degree of identity or correspondence between nucleic acid or amino acid sequences that may or may not share a common evolutionary origin (see, Reeck et al., supra). However, in common usage and in the instant application, the term “homologous”, when modified with an adverb such as “highly”, may refer to sequence similarity and may or may not relate to a common evolutionary origin.

In specific embodiments, two nucleic acid sequences are “substantially homologous” or “substantially similar” when at least about 80%, and more preferably at least about 90%, at least about 95%, or at least about 99% of the nucleotides match over a defined length of the nucleic acid sequences, as determined by a sequence comparison algorithm known such as BLAST, FASTA, DNA Strider, CLUSTAL, etc. Sequences that are substantially homologous may also be identified by hybridization, e.g., in a Southern hybridization experiment under, e.g., stringent conditions as defined for that particular system.

Similarly, in particular embodiments of the invention, two amino acid sequences are “substantially homologous” or “substantially similar” when greater than about 80%, about 90%, about 95% or about 99% of the amino acid residues are identical or similar (i.e., are functionally identical). Preferably the similar or homologous polypeptide sequences are identified by alignment using, for example, the GCG (Genetics Computer Group, Program Manual for the GCG Package, Version 7, Madison Wis.) pileup program, or using any of the programs and algorithms described above (e.g., BLAST, FASTA, CLUSTAL, etc.).

A nucleic acid molecule is “hybridizable” to another nucleic acid molecule, such as a cDNA, genomic DNA, or RNA, when a single stranded form of the nucleic acid molecule can anneal to the other nucleic acid molecule under the appropriate conditions of temperature and solution ionic strength (see Sambrook et al., supra). The conditions of temperature and ionic strength determine the “stringency” of the hybridization. For preliminary screening for homologous nucleic acids, low stringency hybridization conditions, corresponding to a Tm (melting temperature) of 55° C., can be used, e.g., 5× SSC, 0.1% SDS, 0.25% milk, and no formamide; or 30% formamide, 5× SSC, 0.5% SDS. Moderate stringency hybridization conditions correspond to a higher Tm, e.g., 40% formamide, with 5× or 6× SSC. High stringency hybridization conditions correspond to the highest Tm, e.g., 50% formamide, 5× or 6× SSC. SSC is a 0.15M NaCl, 0.015M Na-citrate. Hybridization requires that the two nucleic acids contain complementary sequences, although depending on the stringency of the hybridization, mismatches between bases are possible. The appropriate stringency for hybridizing nucleic acids depends on the length of the nucleic acids and the degree of complementation, variables well known in the art. The greater the degree of similarity or homology between two nucleotide sequences, the greater the value of Tₘ for hybrids of nucleic acids having those sequences. The relative stability (corresponding to higher Tₘ) of nucleic acid hybridizations decreases in the following order: RNA:RNA, DNA:RNA, DNA:DNA. For hybrids of greater than 100 nucleotides in length, equations for calculating Tm have been derived (see Sambrook et al., supra, 9.50-9.51). For hybridization with shorter nucleic acids, i.e., oligonucleotides, the position of mismatches becomes more important, and the length of the oligonucleotide determines its specificity (see Sambrook et al., supra, 11.7-11.8). A minimum length for a hybridizable nucleic acid is at least about 10 nucleotides; preferably at least about 15 nucleotides; and more preferably the length is at least about 20 nucleotides.

In a specific embodiment, the term “standard hybridization conditions” refers to a Tₘ of 55° C., and utilizes conditions as set forth above. In a preferred embodiment, the Tm is 60× C; in a more preferred embodiment, the Tₘ is 65° C. In a specific embodiment, “high stringency” refers to hybridization and/or washing conditions at 68° C. in 0.2× SSC, at 42° C. in 50% formamide, 4× SSC, or under conditions that afford levels of hybridization equivalent to those observed under either of these two conditions.

### Complex Diseases

In both the systemic and organ targeted autoimmune diseases, as well as other complex diseases such as schizophrenia and Alzheimer disease, there is good evidence for a genetic component. In some cases, familial forms of the disease have led to identification of altered genes that are also important in sporadic forms of the disease. For example, Alzheimer disease, the most common form of dementia, can be inherited as an autosomal dominant trait in some families. A study of four large kindreds first demonstrated linkage of early onset-Alzheimer disease with DNA markers on chromosome 21 (87). A number of subsequent studies have localized the AD1 locus to the site of the amyloid precursor protein (APP) gene on chromosome 21q. A review of multiplex Alzheimer pedigrees indicated that the APP locus accounted for 63±11% of those pedigrees, although only a subset of those families have mutations in the APP protein (88). While other genes, including presenilin-2 and apolipoprotein E, have also been associated with Alzheimer disease, it has been suggested by Hardy that the common feature of the many forms of Alzheimer disease is that they all involve altered processing of APP (89).

Genetic factors have been proposed to be involved in the etiology of schizophrenia for many years, but it is only with the advent of the use of microsatellite markers for genome analysis that regions of the genome that might be associated with the disease have been identified. As in Alzheimer disease, large extended pedigrees of families enriched for schizophrenia have been studied to more convincingly identify disease loci. A recent study of thirteen large families using 365 microsatellite markers identified five distinct loci with LOD scores>3.0 in the entire sample or in individual pedigrees (84). None of the specific genes within these loci have been determined.

In the case of autoimmune disorders the diseases can also run in families, and interestingly, some families have members with various autoimmune diseases. For example, a family might have one individual with SLE, another with IDDM, and another with an autoimmune thyroid disease. Genome studies have defined multiple loci that seem to be statistically associated with a diagnosis of one or another of these diseases. Some of these loci seem to be in common to multiple autoimmune diseases (9). There is the concept of “autoimmunity genes” and the idea of threshold. In contrast to single gene diseases, such as cystic fibrosis or sickle cell anemia, where there is one particular mutation or any of a number of alterations in one specific gene, in both systemic and organ targeted autoimmune diseases there is not one locus that is identified as linked to the disease. Rather there are many loci that seem to have a low level association. The current idea of threshold suggests that these loci represent sites of individual variability (not necessarily abnormality) in multiple genes that confer either altered levels of expression or subtly different quality or quantity of function, such that if an individual has a few of the variants that may confer disease susceptibility, they are unlikely to get the autoimmune disease. Comparatively, if they have several gene variants that confer susceptibility, their immune/inflammatory function would be such that they are more likely to develop the autoimmune disease. It has been proposed by others that most of the autoimmune disease susceptibility loci encode genes that are associated with the immune or inflammatory systems (e.g., IL-2, FcR, MHC molecules, cytokines, apoptosis molecules).

Pathogenic agents of autoimmune diseases such as, but not limited, to demethylating drugs and ultraviolet light, have been well studied and evaluated. Several autoantigens and autoantibodies have been characterized, the cytokines that are increased or decreased are known, and the roles of complement activation products and immune complexes have been studied. The mechanism or mechanisms that underlie the etiology of these disease states, however, is currently unclear. Despite the fact that some pathogenic agents are known, the mechanism of action of these agents (e.g. the mechanism by which these agents induce activation of an immune response to self antigens) is not known. Additionally, it is not understood why the targeted antigens in SLE tend to be components of particles that contain both proteins and nucleic acids or how the immune system becomes activated by these particles.

The term “breaking tolerance”, as used herein, is used to address the question as to what triggers an immune response to the relevant autoantigens in each of these disease states. During development, thymocytes that have high affinity for self antigens are removed from the system, and peripheral tolerance mechanisms operate in the mature immune system to discourage activation of self reactivity. T cells specific for some self antigens have probably not been efficiently deleted, but those antigens are likely to be those that are hidden away in “immune privileged” sites, such as the eye and testis, and for that reason an immune response is never generated.

There are features of DNA and RNA that can promote or induce immune responses. The CpG motifs (pairs of C's and G's) are particularly enriched in viral and bacterial DNA and can activate NF-kB and generally act as immune adjuvants. When these motifs are present in mammalian DNA they are usually methylated, resulting in “hiding” the DNA. The effect of the methylation would be to inhibit those motifs that can act as immune adjuvants and, should those motifs be present in a regulatory region of a gene, to inhibit their participation in transcriptional activation. RNA also can activate adjuvant activity that promotes immune system activation. Double stranded RNA can, through somewhat unclear mechanisms, induce the production of interferon-α, which in turn can promote dendritic cell (i.e., antigen presenting cell) function. Either of these events can provide sufficient immune stimulation to inappropriately trigger an immune response.

Another consideration in mechanisms of breaking tolerance is exposure of “cryptic” or altered epitopes. When an antigen or self antigen is processed by an antigen presenting cell, there are characteristic sites of protein cleavage that generate peptides expressed on major histocompatability class molecules to T cells. In the case of self antigens, self peptides are probably presented to thymocytes during development and those with high affinity for that peptide are removed from the system. If the self protein is then presented to T cells in an alternate situation (e.g., in association with another protein), or if the self-antigen is handled in a different manner in the antigen presenting cell, resulting in presentation of a different or altered epitope, the T cell component of the immune response may recognize that antigen.

Possible mechanisms through which self tolerance could be broken would include association of self-antigen with an effective adjuvant, such as DNA enriched in CpG motifs or RNA that can induce interferon-α, or presentation of a self antigen that looks different to the immune system, either because an atypical peptide is presented or because a typical peptide is presented in a different manner or context (like in association with an epitope from an immunogenic peptide). Finally, antigen dose may be important. If the immune system experienced sustained or recurrent exposure of a self-antigen, probably in the presence of an adjuvant activity, that self-antigen may be reacted to.

When proteins, whether foreign to the organism or self-proteins, interact with the immune system of an organism in the absence of pro-inflammatory stimuli, that immune system often fails to respond. In contrast, when a protein is introduced to the immune system together with a substance that triggers inflammation, an “adjuvant”, the properties of antigen presenting cells are altered to facilitate antigen-specific activation of T-cells. In the case of intracellular self-antigens, it is proposed that when the protein components of intranuclear or intracellular particles are associated with non-specific immune stimulants, an antigen-specific immune response to the protein components of that particle might be triggered. Those non-specific immune stimulants, or endogenous adjuvants, might include DNA enriched in CpG motifs, known to trigger activation of the pro-inflammatory transcription factor NF-kB, a protein that mediates tumor necrosis factor (TNFα) transcription, or RNA that can achieve a double stranded conformation and acquire the capacity to induce production of interferon-α. These are all known to induce the maturation and increase the antigen presenting capacity of dendritic cells.

Another concept that has been discussed in the context of “breaking tolerance” is the concept of “altered self”. The idea here is that a self-antigen might appear foreign to the immune system if it achieved a different amino acid sequence or conformation than its typical sequence or structure, if an antigen presenting cell processed the protein in an atypical manner, or if the peptide generated by the antigen presenting cell bound to the groove of MHC molecules in an atypical orientation. Somatic mutations in genes such as p53 are known to induce an immune response to the altered p53. Chromosomal translocations can generate fusion proteins of two genes. Activation of caspases in the setting of apoptosis generates cleavage products of self-proteins that might be capable of immune system activation.

### Systemic Autoimmune Diseases

Prototypical systemic autoimmune diseases include SLE, scleroderma, mixed connective tissue disease, Sjögren's syndrome and other systemic disorders. Epidemiological studies indicate that typical onset of these diseases occurs in the teenage years to the 20's (i.e., post-puberty). Additionally, studies indicate that these diseases affect women in significantly greater numbers than men, in a ratio of about 8-9:1.

These disease states are characterized by generalized immune system activation, but with evidence for antigen-specific induction of T cell-dependent autoantibodies. For example, in SLE autoantibody specificities are very characteristic. Autoantigens include nucleosomes (particles containing histones and DNA); ribonucleoprotein (RNP) particles (containing RNA and proteins that mediate specialized functions in the RNP particle); and double stranded DNA. An example of proteins that mediate specialized functions in the RNP particles is the Sm protein, which has spliceosome function. It is theorized that an inappropriate immune response is initiated in SLE. The response appears to be initiated to a component of one of the intracellular particles, which then spreads to other components of the particle. Tissue damage mostly occurs through actions of the autoantibodies, including activation of the complement system, although antigen-specific T cells also may play a direct role in tissue damage. The tissue damage may be triggered or exacerbated by drugs that may demethylate DNA and by sunlight (e.g. UV light).

### Organ Targeted Autoimmune Diseases

Organ targeted autoimmune diseases include IDDM, MS, autoimmune thyroid disease, RA, pemphigus, psoriasis, polymyositis, dermatomyositis, pemphigoid, vitiligo, primary bilary cirrhosis, chronic active hepatitis, Crohn's disease, ulcerative colitis and pernicious anemia. Epidemiological studies indicate that these diseases have variable onset. Gender distribution studies indicate that some are more common in females, whereas others have a more even gender distribution.

These disease states are characterized by an inappropriate immune response to a self-protein. The response often spreads to include other antigens, notably those that are enriched in the target organ. For example in IDDM, the earliest detectable immune response is directed at the protein glutamic acid decarboxylase (GAD) with later responses directed toward insulin. The self-proteins targeted in some other organ-specific autoimmune diseases include, desmoglein 3 in pemphigus vulgaris, desmoglein 1 in pemphigus foliaceus, myelin oligodendrocyte glycoprotein in MS, tyrosinase related protein in vitiligo, thyroid stimulating hormone receptor in autoimmune thyroid disease, bullous pemphigoid antigen 1 in bullous pemphigoid, and SP100 in primary bilary cirrhosis. There are other complex autoimmune diseases, rheumatoid arthritis for example, in which the relevant autoantigens have not been identified. Antigen-specific T-cells triggered by these antigens mediate tissue damage in the target organ. Cytokines and autoantibodies also may contribute to development of the disease state.

### Long Interspersed Nuclear Elements (LINEs)

As described above, LINEs are believed to be fragments of a nucleotide sequence that has been distributed at many locations throughout the genome, and contain a 5′ regulatory region and two open reading frames (ORF) that can encode two proteins (ORF1 and ORF2). These two ORFs are transcribed into mRNA, which are copied back (or parts of it) into DNA, and the DNA inserted back into the genome.

Thus, a key role for LINEs in driving the increasing sophistication and diversity of the immune system throughout evolution is supported by the heavy load of those elements in the segments of the genome encoding the major histocompatibility (MHC) complex, immunoglobulin heavy and light chains, and T cell receptors. In addition, L1 elements may have been important in the evolution of genomes in general, by generating diverse genomic substrates of sequence modules, along with mutations superimposed on those modules, that could be selected, or not selected, for improved function at the molecule, cell, or organism level. Such a function would justify the maintenance of these potentially damaging genetic elements: they continually build the integrity of the host defense system and the effective function of the organism. Genes that jump into various places in the genome could significantly alter the function of various proteins. Therefore, it is believed that few of these LINEs are capable of doing so. It has been estimated that of the more than 100,000 LINE sequences, there are only approximately 30-60 functional (able to transpose) LINEs in the diploid human genome (43,59). Tight regulatory control of those potentially active elements is likely.

### Expression and Function of L1 Products

In studies on the tissue and cell expression of the products of L1 elements, L1 products have been observed in both germ cells and non-germ cells of testis and ovary, in syncytiotrophoblast cells of the placenta, as well as in breast carcinoma cells (56, 57, 67). The best-studied systems are several teratocarcinoma cell lines, which have been used to define the compartmentalization of the ORF1 p40 in cytoplasmic RNP particles (54). The testis is a fairly well-protected immune privileged site, and germ cells are constantly generated without stimulation of the male immune system. Comparatively, the ovary is more accessible to the immune system, and its products, the ova and shed follicular cells, may be found in various areas within the body such as, but not limited to, the peritoneal cavity. Additionally, eggs are generated episodically, a kinetic pattern which is proposed to be more conducive to immune system triggering (e.g., priming, followed be monthly boosting). While the immune system is somewhat suppressed during pregnancy, if L1 proteins are expressed in the placenta, there might be the opportunity for some immune reactivity to them. The placenta is a target of disease in some lupus patients. Thus, these proteins can play a role in generating diversity in the germ cell, as a supplementary mechanism to crossing-over/recombination. Sex-specific differences in the regulation of L1 gene regulation, as by SOX family proteins for example, may modulate their expression in males more than females. In addition, their limited distribution may mean that they are not so available to effectively induce immune tolerance and if present in sufficiently high levels post puberty, may be able to trigger an immune response to themselves or their associated proteins or nucleic acid. Expressed sequence tags (ESTs) from normal breast tissue also encode ORF1 p40.

Beyond this location of L1 proteins in reproductive organs, there have been a few reports, mostly in mouse literature, showing L1 products in lymphocytes (55). B cells can act as antigen presenting cells when activated, so the B cell could be both a source of L1-derived self antigens as well as the cells that present those antigens to T cells, thus initiating an autoreactive immune process. One paper has suggested that L1-containing particles (proteins and nucleic acid) can assist in repairing double stranded DNA breaks (60). Of the 3 unique processes that B cells undergo, VDJ recombination, Ig class switching, and somatic hypermutation, all three require cleavage of double stranded DNA. Without wishing to be bound by theory, it is possible that the protein products of L1 elements might be recruited to perform a physiologic function that is DNA repair related. Interestingly, the classic autoantigen targeted by autoantibodies in SLE is a double stranded DNA. If the immune system were exposed to double stranded DNA in association with L1 proteins, to which the immune system is not tolerant, along with the adjuvant activities (such as interferon-″) induced by the presence of L1 RNA, the double stranded DNA may be targeted by the immune response. L1 products may also be present at sites of inflammation. Expression of L1 ORF1 p40 mRNA and protein has been observed in RA synovial tissue and has been suggested to have the capacity to trigger intracellular kinase pathways that mediate inflammation (58).

Several of these elements have been roughly localized on their appropriate chromosomes. Some of the L1 elements are polymorphic (43,44). Interestingly, L1 sites of a small population study showed that the African American ethnic group had the highest frequency of a particular L1 element located to chromosome Iq (43). Beyond individual variability in the presence or absence of a given L1 element, the 5′ regulatory region as well as the 5′ part of ORF1 are quite variable. So there might be base differences from one individual to another that would affect the efficiency of transcription or function of the encoded protein product of ORF1 (the p40 protein).

While the human 5′ regulatory region of the gene encoding ORF1 is a single stretch of nearly 900 bp, the mouse 5′ regulatory region comprises variable numbers of tandem repeats of a CpG island, along with a short tether that anchors the modules to the ORF1 coding sequence. The 5′ 40% of mouse and human ORF1 sequences are unrelated. Although this application focuses on human diseases, disease genes, and susceptibility and triggers for human disease, it is predicted that murine L1 elements will be found near murine susceptibility loci as preliminarily found in human chromosomes.

L1 proteins are usually not expressed. Therefore, there must be reasonably effective controls in place that inhibit transcription of L1's. One potential mechanism is the methylation of CpG motifs in the 5′ regulatory region. There are studies indicating the importance of these motifs in regulation of L1 expression. It is of interest that many of the drugs that typically induce lupus have the effect of demethylating DNA. Moreover, a murine model of lupus has been established in which treatment of mouse lymphocytes with 5-azacytidine can result in the capacity of those lymphocytes to induce lupus. Similarly, there are suggestions that UV light, a classic exogenous trigger of disease exacerbation in SLE, can promote gene transcription of L1 elements. In addition, the inhibitory capacity of the SRY male-specific transcription factor in regulation of L1 transcription suggests that L1 may be more stringently regulated in males compared to females.

### Role of L1 Retrotransposons in Human Disease

Documentation of functional activity of L1 elements has been provided by instances of gene inactivation following insertion of a retrotransposon (61-64). Such genetic diseases have been documented in man, mice, and dogs (36). Among the first and best studied germline insertions are those into the factor VIII and dystrophin genes of individuals with sporadic (i.e., no family history) hemophilia and muscular dystropy, respectively (61,64). Therefore, it was proposed that the L1 transposed into the previously normal gene, disrupting its expression. Kazazian described insertion of the 3′ end of L1 into exon 14 of the factor VIII gene in two unrelated patients with hemophilia (61).The limitation of the transposed element to its 3′ portion is typical; it is a rare L1 sequence in which the 5′ segment is not truncated. The Fas mutation that accounts for the lupus accelerating phenotype in MRL/lpr mice represents an insertion of a retrotransposon into that gene (65). These rare instances of gene disruption are striking but may not represent the most significant impact of L1 elements in human disease. Some instances of chromosomal translocation in malignancy are associated with insertion of a partial or full-length L1 element into one of the transposed gene partners.

Most relevant to the pathogenesis of complex diseases, particularly autoimmune diseases, transcriptionally active L1 elements may provide the trigger for disease initiation. At least eight mechanisms can be postulated through which retrotransposons could mediate human disease: 1) gene disruption; 2) gene transposition; 3) induction of mutations in nearby genes; 4) altered transcriptional regulation of a gene by a nearby L1 element; 5) altered splicing or translation of a mRNA based on inclusion of L1 elements in its intronic or untranslated segments; 6) induction of an immune response to the transcribed and translated products of the retrotransposon; 7) induction of an immune response to co-transcribed genes adjacent to a retrotransposon; 8) induction of an immune response to proteins, DNA, or RNA physically associated with L1 DNA, RNA or protein.

The present invention discloses that the complex pattern of multiple SLE genetic susceptibility loci identified in microsatellite total genome studies can represent replicate copies of one family of genes, the L1 retrotransposon elements, rather than many discrete genes. This model can also apply to other systemic autoimmune diseases, as well as complex diseases not known to be autoimmune in nature. While polymorphisms in individual genes that regulate immune system activity or tissue response may play a role in disease expression, the bulk of SLE genetic susceptibility can be attributable to variable expression or efficiency of transcription of members of the L1 element family. The RNA and protein products of those L1 elements would act in a threshold manner to trigger immune reactivity to intracellular RNP particles, co-transcribed gene products, and possibly to double stranded DNA breaks, RNA, or proteins to which L1 products bind. The present invention further identifies potential therapeutic targets.

L1 retrotransposon elements or their products can be the primary triggers of the antigen-specific immune system activation that results in the inflammatory and tissue destructive manifestations of complex diseases such as SLE. Although the individual whose genome is enriched in full-length L1 elements capable of retrotransposition will be particularly susceptible to these diseases, successful transposition would not be a requirement for disease induction. If the L1 coding region is transcribed into mRNA and that RNA into ORF1 p40 protein, those events might be sufficient to trigger complex disease, the prototype being SLE. The presence of the specific L1 RNA, with sequence features common to RNA viruses, along with the p40 protein in cytoplasmic RNP particles, also might trigger autoimmunity through a compound mechanism. Expression of p40 is highly restricted in both time and location (56,57). In view of this limited expression, central immune tolerance to p40 might be only partial, resulting in an immune system ready for activation should antigen load pass a threshold. The presence in a particle of RNA with the sequence features of viral RNA might stimulate cellular production of interferon-α, a cytokine that provides a mechanistic bridge between innate and adaptive immunity. The effect would be an immune system milieu supportive of an antigen-specific response to components of the RNP particle itself, as well as any associated proteins or nucleic acid fragments. The chronic and recurrent immune response stimulated in this way would result in the spectrum of pathogenic autoantibodies typical of SLE, as well as the secondary manifestations of immune system activation and dysfunction that are well described (69, 70). An additional method of induction of autoimmune disease by retrotransposons, described in the fourth mechanism above also may have some role in these diseases. Increased transcription of a gene may be mediated by effects of a nearby L1 element on the promoter region of the gene. The increased production of that gene product might be sufficient to cross a threshold for induction of an immune response under appropriate immunostimulatory conditions. Another related method of induction of autoimmune disease is described in the seventh mechanism described above. Transcriptionally competent L1 elements might activate an immune response to the products of nearby genes. Transcription of nearby genes can generate “readthrough” transcripts that include L1 sequences, and conversely, transcription of the LINEs may activate or modulate transcription of genes 3′ to the L1 element. In either case, the presence of L1 nucleotide sequences and p40 protein together with a normal gene product might trigger immune reactivity to that gene product.

Those individuals with highly active L1's that encode ORF1 and ORF2 proteins with perfect or near-perfect sequence (meaning the proteins translated will function effectively) may be susceptible to random insertions into genes, disrupting the function of those genes and causing significant disease mediated by impaired function of a single gene (as in the noted case of hemophilia). Such individuals may also be more likely than others to produce L1 RNA transcripts, along with ORF1 and/or 2 proteins, that cluster together, along with other proteins, in RNP particles in whatever cells are most likely to make these products, such as ova and follicular cells in the ovary, cells in the testis, placental trophoblast cells, breast tissues, and possibly B (and/or T) lymphocytes. The RNA, known to fold into 3-dimensional conformations, and with sequence features with some similarities to viral RNA, may trigger production of interferon, an immunostimulant. The DNA copied from that RNA will be rich in CpG motifs with adjuvant properties. If the immune system (CD4+ T cells) becomes exposed to these L1 proteins, L1 RNAs, and/or L1 DNAs, along with the adjuvant factors (interferon, etc.), “breaking tolerance” and triggering an immune response to any or all of the components of those particles will be set up. The immune response is known to undergo determinant spreading from an initial triggering epitope in a particulate antigen to other epitopes. As such a response developed, a spectrum of autoantibodies would emerge that are characteristic of those seen in patients with SLE. This autoantibody response might also include some directed toward double stranded DNA, targeted because it associates with the L1 products at sites of DNA cleavage, or proteins. Those individuals with genetic susceptibility to SLE would correspond to those individuals with either more L1 elements in their genome and/or more functional (transcribable) L1 elements. Those individuals could be identified by generating a map of the location of high fidelity (with DNA sequence very similar to or identical to the characterized active L1 elements), full-length (able to encode ORF1 and/or ORF2 protein) L1 elements, sequencing the DNA of an individual in those regions of the genome, and determining the presence of the elements, their fidelity to consensus, and whether they are full-length (with full regulatory region, ORF1 and ORF2). The location of such L1 elements on chromosome 1q and 16 are proximate to several of the markers that have been identified for lupus susceptibility loci. Individuals with L1 elements that are located in intronic segments of genes would also be identified by mapping such elements and the genes they are associated with and then sequencing or otherwise characterizing the DNA of the individual. The sequencing and DNA analysis can be performed using any method known in the art such as, polymerase chain reaction, SSCP, or Southern blotting.

Additionally, L1 elements which confer susceptibility may be those L1 genes situated near genes such that they either confer increased transcription immunogenicity on the nearby gene or confer increased immunogenicity on the nearby gene product. If an L1 element is sufficiently intact to initiate gene transcription, but not of sufficient fidelity to the consensus sequence to produce functional ORF1 and 2 proteins, it might produce a transcript that is a hybrid of the L1 transcript and the neighboring gene transcript. If the host gene mRNA is translated into protein and remains associated with the L1 transcript, tolerance to the gene product might be broken by virtue of the induction of adjuvant activity by the L1 transcript. Or conversely, activation of the host gene in the normal physiologic course of events, or in the setting of infection or stress, would result in transcription of L1 mRNA, along with the host gene mRNA. Either way, host gene products would be physically associated with potentially immunogenic L1 products. From the data obtained, these L1's are of fairly high fidelity (usually 85 to 9596%), but probably not sufficiently high fidelity to represent a fully active element. This level of sequence fidelity may reflect competence sufficient to initiate transcription but not to produce functional proteins. These locations can be mapped and individual DNA samples tested to determine the presence and the degree of fidelity and intactness of these L1 sequences.

### Methods for Identifying Genes and Gene Products Involved in Human Disease

The present invention provides a method that allows the identification of genes and gene products that are candidates for involvement in human disease. In view of the important role that L1 elements have likely played in the evolution of the human and other genomes, and the requirement that a functional L1 element be full-length (including the entire or nearly entire approximately 6000 nucleotide sequence) and capable of being transcribed and translated into functional protein, the identification in the genome of the location of full-length L1 elements of high level identity to the consensus sequence of a known functional L1 element can be used to identify genes relevant to human disease. Moreover, the identification of genes or mRNAs in which full-length L1 elements are included in intronic or untranslated segments can be used to predict candidate disease genes, mRNAs, and proteins important in human disease. This invention is based on the hypothesis that individual genomic variability can be reflected in disease, and the location of L1 elements can provide an important predictor of the sites of disease-relevant genomic variability.

In general, the method can be exercised by cataloguing the location of L1 elements in the genome, without prior information regarding disease susceptibility loci, or it may be exercised by studying a segment of the genome in a region encompassing that locus. The method in either case involves the comparison of the sequence of a known segment of DNA with the DNA sequence of the 5′ segment of a known functional L1 element. The known segment of DNA may be derived from a contig, a bacterial artificial chromosome (BAC), or a gene sequence published in a publicly available database or any proprietary DNA sequence of more limited availability. In some cases, RNA sequences may also be useful for analysis.

The L1 sequence used for comparison can be derived from a publicly available sequence of a full-length L1 element that has been demonstrated to be capable of transposition. As the genome is composed of thousands of fragments of L1 elements derived from the 3′ end of the consensus sequence, it is cumbersome to conduct comparison searches of the entire L1 sequence with a test genome sequence. The method is therefore most effectively conducted by use of the 5′ region of a consensus L1 element.

For example, in Examples 8-11 herein, approximately the most 5′ 900 nucleotides of the L1 sequence located on chromosome 1q, termed LRE2 and published in the GenBank database under accession number U09116, was used in Pairwise BLAST “BLAST 2 sequences” searches against one or a series of contigs, BAC clones, or any published DNA sequence. The method is also effective using shorter segments of the 5′ region of the consensus L1 sequence. The important aspect of the method is that the most 5′ segment of the sequence, whether it be the most 5′ 100 or the most 5′ 900 bases, is used.

Matches with the test DNA segment are scored as positive if they meet either of three criteria: 1) the tested DNA sequence has about 97%, about 98%, about 99%, or about 100% identity to the 5′ region of the consensus L1 element, specifically nt 1-884 of U09116. and is located within about 200,000, more preferably about 100,000 bases, and even more preferably about 50,000 bases of a gene2) the tested DNA sequence includes the 5′ region of a consensus L1 element in an intron or untranslated segment. 3) In addition, full-length high fidelity are scored positive if their 5′ sequence is about 98%, about 99%, or about 100% identical to nt 1-883 of the L1 consensus sequence from U091 16, even if they are not located in close proximity to a gene or predicted gene. For the first criteria, the selection of 100,000 bases for the margins of proximity of the high fidelity L1 element to a neighboring gene is assigned arbitrarily based on studies indicating that gene regulation can be modified by sequences as distant as 100,000 bases, but these criteria to do not strictly limit the method to that DNA distance (90). The distance between the first nucleotide of the L1 element and the first nucleotide of the susceptibility gene can be measured as bp. A potential disease gene is identified as being less than about 200,000 bp, preferably less than 100,00 bp, and most preferably less than about 50,000 bp from the 5′ end of the L1 element. The second criterion does not require that the L1 sequence must be of 98, 99, or 100% sequence identity to the consensus L1 sequence. Typically, full-length L1 elements included in intronic gene segments range from 80-99% fidelity to the consensus L1 sequence. It should be noted that occasionally L1 sequences do not extend to the very 5′ extent of the consensus sequence, but may rarely lack up to the most 5′ 10 bases.

Once a list of genes proximal to a full length high fidelity L1 element, or containing an L1 element in their intronic regions, has been generated, those genes can then be further explored for a role in disease pathogenesis, as sites of individual variability that confers susceptibility to disease, as participants in disease-relevant molecular pathways, and as potential targets for therapy.

### Methods for Determining Disease Susceptibility or Diagnosing a Complex Disease

In order to determine susceptibility to, or diagnose, a complex disease in an individual, the presence on a particular chromosome in an individual's genome of an L1 element that is capable of being transcribed can be assessed. The presence of an intact 5′ regulatory region in the context of the adjacent DNA sequence specific to that chromosomal location can be determined. Some L1 elements will either be present or absent. Additionally, some L1 elements may be present but contain variable nucleotides (nt) in different individuals.

PCR and nested PCR techniques may be used to amplify sequences of interest. Nested primer sets for PCR are designed using the nucleotide sequence that includes approximately 800 nt 5′ of the initiation of the 5′ regulatory region of the L1 element and the first approximately 50 nt in the L1 regulatory region. DNA can be isolated from a variety of sources including, but not limited to, peripheral blood cells or another cell source, from a patient with an autoimmune or complex disease or who may be suspected to be susceptible to or possibly developing an autoimmune or complex disease. The presence of a PCR amplified product can then be associated with the presence or absence of an autoimmune disease in a population of patients, or in a subpopulation of patients expressing particular clinical or laboratory features of the disease, and compared to the presence of a similar band in control subjects. The same method may also be used to study individuals suspected to be susceptible to or possibly developing a complex disease that is not traditionally considered an autoimmune disease. Examples of such diseases are Alzheimer disease and schizophrenia, but the method is not limited to those diseases.

The presence or absence of an L1 element containing an intact 5′ regulatory segment at a particular chromosomal site also can be determined with Southern blot analysis under conditions of high stringency using well known techniques. As in the case of PCR and nested PCR, the presence of the 5′ regulatory region of the L1 element of interest can be determined by the presence of a band indicating reaction of the labeled probe with the particular DNA segment of interest.

In some cases the presence or absence of the 5′ regulatory region of the L1 element will be observed, in other cases, the 5′ regulatory element will be present, but it will have nt variations in the study individual compared with DNA from healthy or disease control individuals. These nt variations can be detected by direct sequencing of the products of either the initial PCR reaction described above, or the nested PCR reaction. The PCR product can either be directly sequenced, using an automated sequencing instrument, or the PCR product can be subcloned into a cloning vector, positive clones picked, plasmid DNA prepared and directly sequenced. Alternative approaches to mutation detection can also be used to identify individual differences in nt sequences in the amplified PCR product. The presence or absence of nucleotide changes at a particular site in the 5′ regulatory region can be studied for association with a diagnosis of autoimmune or other complex disease, or clinical or laboratory features of the disease.

Once it has been determined that an individual contains in their genome an L1 element that is located at a particular chromosomal location, the presence in that L1 element of a full-length 5′ regulatory region and the approximately 5′ one third of ORF1 that are of high fidelity to a consensus L1 sequence can be determined using a 5′ primer and a 3′ primer that is located at the approximate mid-point of ORF 1. The PCR product can be directly sequenced or subcloned and sequenced as described above. The presence of an L1 element at the particular chromosomal location that is full-length and/or is of high fidelity compared to a consensus sequence can be determined using DNA isolated from cells or tissue of an individual with or suspected to have or to be susceptible to an autoimmune disease, and compared to DNA from a healthy or control individual. Other approaches can be taken to identify individual nt differences in these regions between and among DNA from different individuals. For example, high pressure liquid chromatography can be used to determine heteroduplex formation between two strands of DNA spanning the 5′ regulatory region and 5′ segment of ORF1 of an L1 element located at a particular chromosomal site in order to identify nt differences between the DNA strands of two individuals.

The presence of an L1 element within the regulatory region or in an intron of a gene can modify the expression of that gene. If that gene product is important in the immune or inflammatory pathways, altered expression of the gene product can contribute to autoimmune disease. Alternatively or additionally, the presence of an L1 element in a location proximate to a gene or within the introns of a gene may result in generation of an RNA product that includes RNA sequences encoded by the L1 element as well as RNA sequences encoded by the neighboring or surrounding gene. Such an RNA transcript may promote an autoimmune reaction to the product of the neighboring or the surrounding gene. The presence of an L1 element within or near a gene can be determined by identifying the location of that gene of interest, identifying a DNA sequence in the Genbank that includes an L1 sequence within or proximate to the gene of interest, and identifying PCR primers that will amplify a segment of that L1 element in the context of the chromosomal site in which it is located. DNA from an individual can then be assessed for the presence of that L1 element, or for the particular sequence of that L1 element, using PCR or nested PCR, Southern blots, direct sequencing, or other techniques.

The presence of an insertion in an individual with an autoimmune disease, or one who is suspected to be susceptible to developing an autoimmune disease, can be detected by isolating DNA from blood or tissue cells, or any other DNA source, from that individual and designing PCR primers that will amplify the L1 insertion in the context of the chromosomal locus of interest. The presence of a PCR amplified product can then be associated with the presence or absence of an autoimmune disease in a population of patients, or in a subpopulation of patients expressing particular clinical or laboratory features of the disease, and compared to the presence of a similar band in control subjects. Such an L1 element can also be identified using ³²P-labeled DNA probes in a Southern blot.

### Transcription of L1 Elements

Transcriptional activity of L1 elements can be assessed by techniques that detect and quantitate mRNA encoded by the L1 element ORF1 or ORF2. Production of the protein products of L1 elements can be detected and quantified by techniques that identify a specific protein. Cells, tissues or body fluids (e.g., blood, serum, saliva, urine, tears, sweat, synovial fluid, cerebrospinal fluid and the like) can be isolated from an individual with an autoimmune disease or suspected to be susceptible to developing an autoimmune disease in order to measure L1 encoded mRNA or protein. In situ hybridization can also be used to detect the mRNAs encoded by L1 elements. In some cases, it may be desirable to induce the expression of L1 mRNA products by treating an individual's cell sample, such as peripheral blood mononuclear cells with an agent that stimulates the transcription of L1 mRNA, including but not limited to 5-azacytidine.

Detection of the protein products of L1 elements, either ORF1 or ORF2 gene products, can be used to indicate the presence in cells, tissue, or body fluids of potential immune system triggers that can induce or exacerbate autoimmune disease. Proteins can be detected by several techniques well known to those of ordinary skill in the art, including immunoprecipitation or Western blot using polyclonal or monoclonal antibodies to the ORF1 or ORF2 products, immunofluorescence or flow cytometry to detect intracellular or cell surface expression of these proteins, immunohistochemistry to detect the proteins in tissue samples, or ELISA to detect L1-encoded ORF1 or ORF1 proteins in plasma, serum, or other body fluids. In some cases, it may be desirable to isolate cells from an individual, as from peripheral blood, and treat those cells with a demethylating agent such as 5-azacytidine or an agent that promotes histone acetylation before isolation of proteins.

Characterization of the nucleotide and protein components of ribonucleoprotein particles can be performed to detect the presence of potentially immunostimulatory L1 products, L1 protein products that can serve as autoantigens, or gene sequences that are expressed in association with L1 products and the protein products of which might become immunogenic when expressed in association with those L1 products. Ribonucleoprotein particles can be isolated from cells derived from an individual suspected of having autoimmune disease (71). The presence of L1 mRNA components in the ribonucleoprotein particles can be detected by generating cDNA followed by PCR amplification using specific primers, or unknown mRNA sequences can be characterized by generating cDNA, followed by direct sequencing. Such RNA transcripts of unknown sequence within ribonucleoprotein particles may identify RNA sequences encoded by genes neighboring or surrounding L1 elements and their protein products may represent putative autoantigens.

Patients with autoimmune disease, particularly those with SLE, often make antibodies with specificity for nucleotide or protein components of intracellular particles, including ribonucleoprotein particles. The presence of autoantibodies specific for L1 DNA or mRNA sequences or for L1 protein products may indicate a diagnosis of autoimmune disease. Detection of a change in the titer or level of those antibodies may be associated with a change in the clinical disease activity of the patient. Serum autoantibodies specific for L1 products can be detected by the techniques of ELISA or immunoblot (72), or other newer techniques such as autoantigen-coupled beads or antigen microarray, with patient serum used to detect the L1 DNA, RNA or protein products, or by immunoprecipitation (72), in which the patient serum is used to precipitate cellular components containing L1 products, or purified L1 products.

### Identifying L1-Elements—Experimental Techniques

This section describes various specific embodiments of the methods of the invention, and includes techniques for identifying L1 elements, their transcription products, and translation products. The L1 sequence found on chromosome 1q25, at approximately 184.5M bases from the 1p telomere, serves as an example of these approaches.

### Identification of High Fidelity L1 Elements With Intact 5′ Regulatory Segments

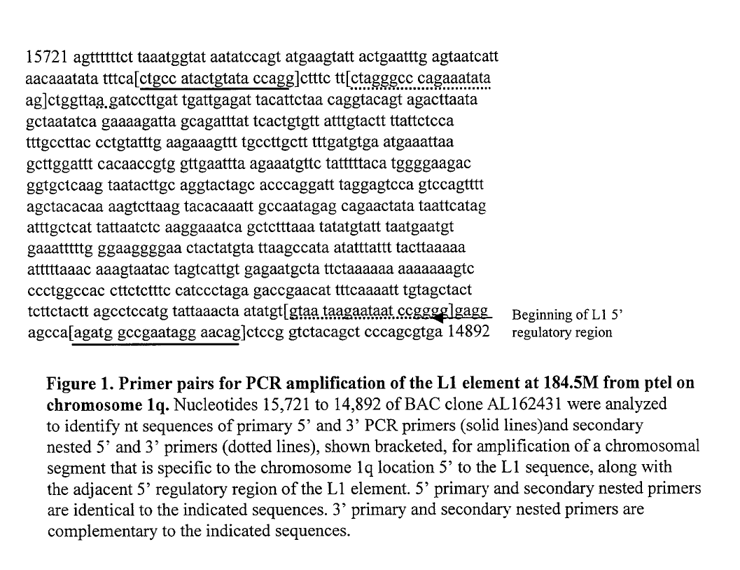

A bacterial artificial chromosome (BAC) clone that includes the L1 element on chromosome 1q25, at approximately 184.5M bases from the 1p telomere, is identified by Genbank accession number AL162431. This sequence is also contained within the contig with GenBank accession number NT_{—}004552. Nested primer sets for PCR are designed using the nucleotide sequence that includes approximately 800 nt 5′ of the initiation of the 5′ regulatory region of the L1 element (the beginning of the L1 5′ regulatory region is considered to be located at nucleotide 14,948 in clone AL162431) and the first approximately 50 nucleotides in the L1 regulatory region (FIG. 1). For example, DNA can be isolated from peripheral blood cells, or another cell source, from a patient with an autoimmune disease, with a family member with an autoimmune disease, or who may be suspected to be susceptible to or possibly developing an autoimmune disease. DNA can also be isolated from blood or another source of cells from a healthy control individual or from an individual with a non-autoimmune disease. In this example, for BAC clone AL162431, a 5′ primer of sequence

CTG CCA TAC TGT ATA CCA GG (SEQ ID NO:5)

identifying a region of chromosome 1q that is 5′ of the L1 5′ regulatory region, and a 3′ primer of sequence

CTG TTC CTA TTC GGC CAT CT (SEQ ID NO:6)

identifying a segment of the L1 5′ regulatory region, can be used to amplify the DNA segment spanning nt 14,927 and 15,656. The PCR product is run on an agarose gel and the presence or absence of a band, representing the product of the PCR reaction, observed. The specificity of the PCR amplification can be further increased by performing a nested PCR reaction, in which the PCR product from the first reaction is excised from the gel, passed through a spin column to remove the first pair of primers, and the product then used as a template in a second PCR reaction that uses primers internal to the first set. For example, a 5′ internal primer of sequence

CTA GGG CCC AGA AAT ATA AG (SEQ ID NO:7)

and a 3′ internal primer of sequence

CCC CGG ATT ATT CTT ATT AC (SEQ ID NO:8)

can be used to amplify the first PCR product (FIG. 1). The resulting product, corresponding to nucleotides 15,619 to 14,946 of BAC clone AL162431, is run on an agarose gel, and the presence or absence of a product observed. The presence of a PCR amplified product can then be associated with the presence or absence of an autoimmune disease in a population of patients, or in a subpopulation of patients expressing particular clinical or laboratory features of the disease, and compared to the presence of a similar band in control subjects.

### Southern Blot Analysis

Other techniques can be used to determine the presence or absence of an L1 element containing an intact 5′ regulatory segment at a particular chromosomal site. For example, the primers described above can be used to amplify the segment that includes the chromosomal region 5′ of the L1 as well as a portion of the 5′ regulatory region of the L1 element. This PCR product can be labeled with ³²P and used as a probe to determine the presence of the complementary DNA fragment in the genome of an individual. DNA is isolated from the individual, and run on an agarose gel after digestion with a restriction enzyme, and then the DNA probed with the ³²P-labeled DNA fragment. As in the case of PCR and nested PCR, the presence of the 5′ regulatory region of the L1 element of interest can be determined by the presence of a band indicating reaction of the labeled probe with the particular DNA segment of interest.

### Direct Sequencing of PCR Products

While in some cases the presence or absence of the 5′ regulatory region of the L1 element will be observed, in other cases, the 5′ regulatory element will be present, but it will have nt variations in the study individual compared with DNA from healthy or disease control individuals. For example, the two BAC clones that identify a particular DNA region may contain nt variations. These nt variations can be detected by direct sequencing of the products of either the initial PCR reaction described above, or the nested PCR reaction. The PCR product can either be directly sequenced, using an automated sequencing instrument, or the PCR product can be subcloned into a cloning vector, positive clones picked, plasmid DNA prepared and directly sequenced. Alternative approaches to mutation detection can also be used to identify individual differences in nt sequences in the amplified PCR product. The presence or absence of nucleotide changes at a particular site in the 5′ regulatory region can be studied for association with a diagnosis of autoimmune disease, or clinical or laboratory features of the disease.

### Sequencing of ORF1 L1 Elements Identified at Particular Chromosomal Locations

Once it has been determined that an individual contains in their genome an L1 element that is located at a particular chromosomal location, the presence in that L1 element of a full-length 5′ regulatory region and the approximately 5′ one third of ORF1 that are of high fidelity to a consensus L1 sequence can be determined using the 5′ primer described above (identifying the non-L1 chromosome-specific sequence) and a 3′ primer that is located at the approximate mid-point of ORF 1. The PCR product can be directly sequenced or subcloned and sequenced as described above. The presence of an L1 element at the particular chromosomal location that is full-length and/or is of high fidelity compared to a consensus sequence can be determined using DNA isolated from cells or tissue of an individual with or suspected to have or to be susceptible to an autoimmune disease, and compared to DNA from a healthy or control individual. Other approaches can be taken to identify individual nt differences in these regions between and among DNA from different individuals. For example, high pressure liquid chromatography can be used to determine heteroduplex formation between two strands of DNA spanning the 5′ regulatory region and 5′ segment of ORF1 of an L1 element located at a particular chromosomal site in order to identify nt differences between the DNA strands of two individuals (83).

### L1 Elements as Disease Genes or Autoantigens

L1 elements inserted within or near genes may be implicated in the pathogenesis of an autoimmune disease or may themselves serve as autoantigens in an autoimmune disease. The presence of an L1 element within the regulatory region or in an intron of a gene may modify the expression of that gene. If that gene product is important in the immune or inflammatory pathways, altered expression of the gene product can contribute to autoimmune disease. Alternatively or additionally, the presence of an L1 element in a location proximate to or within a gene may result in generation of an RNA product that includes RNA sequences encoded by the L1 element as well as RNA sequences encoded by the neighboring or surrounding gene. Such an RNA transcript might promote an autoimmune reaction to the product of the neighboring or surrounding gene. Alternatively or additionally, the presence of an L1 element in or near the regulatory element of a nearby gene may alter the transcription of that gene, resulting in increased production of the gene product, and altered capacity to induce immune system activation. In addition, the presence of and L1 element in the intron or untranslated region of a gene may alter the splicing, mRNA stability, or translation of the mRNA or alter the folding or degradation of the encoded protein. The presence of an L1 element within or near a gene can be determined by identifying the location of that gene of interest, identifying a DNA sequence in the Genbank that includes an L1 sequence within or proximate to the gene of interest, and identifying PCR primers that will amplify a segment of that L1 element in the context of the chromosomal site in which it is located. DNA from an individual can then be assessed for the presence of that L1 element, or for the particular sequence of that L1 element, using PCR or nested PCR, Southern blots, direct sequencing, or other techniques.

For example, at least three BAC clones published in the Genbank include the DNA sequence of the region on chromosome 1q that encodes members of the family of receptors for the Fc segment of immunoglobulin (FcR), as well as several other genes including ATF6. BAC clone AL359541, located approximately 162.3M bases from ptel, contains an L1 insertion in an intron of the FcR/ATF6 locus that includes portions of the 5′ regulatory region, situated in the 3′ to 5′ orientation within the locus. Another clone, AL391825, contains a more complete L1 sequence overlapping the ATF6 gene. Other BAC clones, such as AC027205 do not contain this L1 sequence. The presence of this L1 insertion in an individual with an autoimmune disease, or one who is suspected to be susceptible to or developing an autoimmune disease, can be detected by isolating DNA from blood or tissue cells, or any other DNA source, from that individual and designing PCR primers that will amplify the L1 insertion in the context of the chromosomal locus of interest. The PCR product is run on an agarose gel and the presence or absence of a band, representing the product of the PCR reaction, observed. The specificity of the PCR amplification can be further increased by performing a nested PCR reaction, in which the PCR product from the first reaction is excised from the gel, passed through a spin column to remove the first pair of primers, and the product then used as a template in a second PCR reaction that uses primers internal to the first set. The presence of a PCR amplified product can then be associated with the presence or absence of an autoimmune disease in a population of patients, or in a subpopulation of patients expressing particular clinical or laboratory features of the disease, and compared to the presence of a similar band in control subjects. Such an L1 element can also be identified using ³²P-labeled DNA probes as in a Southern blot after digestion of DNA with a restriction enzyme. When such an insertion of an L1 element is identified, or when an L1 element is identified proximate to a gene of interest, or a full-length high fidelity L1 element is identified of 98%, 99%, or 100% identity to the L1 consensus sequence regardless of its location, the specific nucleotide sequence of that element can be determined by sequencing the PCR product, subclones of that PCR product, or products that include DNA segments adjacent to the 5′ regulatory region of the L1 element.

### Identification of mRNA and Protein Products of L1 Elements

Transcriptional activity of L1 elements can be assessed by techniques that detect and quantify mRNA encoded by the L1 element ORF1 or ORF2. Production of the protein products of L1 elements can be detected and quantitated by techniques that identify a specific protein. Cells, tissues or body fluids can be isolated from an individual with an autoimmune disease or suspected to be susceptible to or developing an autoimmune disease in order to measure L1 encoded mRNA or protein. Total RNA or poly-A RNA is isolated from the sample, cDNA generated, and specific primers used to amplify the L1 mRNA. As sequences from the 3′ end of L1 elements are often transcribed as “readthrough” transcripts in association with mRNA encoded by other genes, it is most effective to use primer sets that amplify the 5′ regulatory region or 5′ region of the ORF1 product. The presence or absence of a band representing the PCR product can be visualized after running the product on an agarose gel. To more quantitatively assess the mRNA products of L1 elements, a quantitative “mimic” PCR can be performed in which composite mimic primers are designed by incorporating a L1 ORF1 (or ORF2) sequence (from Genbank Accession #U09116) along with a v-erbB fragment provided in a PCR mimic kit (Clontech, Palo Alto, Calif.). For competitive PCR, 1 ml of cDNA, 1 ml of each of 10-fold dilutions of the MIMIC (from 5 to 20 attomoles/ml), 0.5 μl of specific primers, and 22.5 ml of PCR super mix (Life Technologies, Gaithersburg, Md.) are combined and PCR carried out in a thermocycler by denaturing at 94° C. for 45 sec, annealing at 55° C. for 45 sec, and with extension at 72° C. for 1 min. The dilution of mimic which produces a band of equal intensity to that of target DNA is determined. The expression of L1 ORF1 or ORF2 mRNA can also be detected by real-time PCR or by northern blot. In situ hybridization can also be used to detect the mRNAs encoded by L1 elements. In some cases, it may be desirable to induce the expression of L1 mRNA products by treating an individual's cell sample, peripheral blood mononuclear cells for example, with 5-azacytidine or other agents that promote demethylation of DNA prior to isolation of RNA or poly-A RNA. Peripheral blood mononuclear cells can be incubated for 24 to 48 hours with 1-5 mM 5-azacytidine, in the presence or absence of a lymphocyte stimulant such as anti-CD3 and anti-CD28 monoclonal antibodies, RNA or poly-A RNA isolated from the cells, and then competitive mimic PCR or real time PCR performed as described to quantitate L1 ORF1 or ORF2 mRNA. Other agents that promote histone acetylation may also be effective in inducing expression of L1 products.

Detection of the protein products of L1 elements, either ORF1 or ORF2 gene products, can be used to indicate the presence in cells, tissue, or body fluids of potential immune system triggers that can induce or exacerbate autoimmune disease. Proteins can be detected by several techniques, including immunoprecipitation or Western blot using polyclonal or monoclonal antibodies to the ORF1 or ORF2 products, immunofluorescence or flow cytometry to detect intracellular or cell surface expression of these proteins, immunohistochemistry to detect the proteins in tissue samples, or ELISA to detect L1-encoded ORF1 or ORF1 proteins in plasma, serum, or other body fluids. In some cases, it may be desirable to isolate cells from an individual, as from peripheral blood, and treat those cells with a demethylating agent such as 5-azacytidine (commercially available from Sigma, St. Louis, Mo.) or an agent that promotes histone acetylation (such as suberoylanilide hydroxamic acid or tricostatin A₁, the latter commercially available from Sigma) before isolation of proteins.

### Identification of L1 Elements and Products in Ribonucleoprotein Particles

This section describes the identification of mRNA and protein products of L1 elements, and associated gene sequences, in ribonucleoprotein particles. Characterization of the nucleotide and protein components of ribonucleoprotein particles can be performed to detect the presence of potentially immunostimulatory L1 products, L1 protein products that can serve as autoantigens, or gene sequences that are expressed in association with L1 products and the protein products of which might become immunogenic when expressed in association with those L1 products. Ribonucleoprotein particles can be isolated from cells derived from an individual suspected of having autoimmune disease by preparing cellular extracts and then centrifuging that preparation at 160,000 g for 2.5 h. The protein components of those particles can be characterized by resolving the proteins on a gel, transferring the proteins to a membrane, and then immunoblotting with an antibody specific for predicted protein components. To identify unknown protein components, a band can be excised from the gel and the amino acid sequence determined. The presence of L1 mRNA components in the ribonucleoprotein particles can be detected by generating cDNA followed by PCR amplification using specific primers, or unknown mRNA sequences can be characterized by generating cDNA, followed by direct sequencing. Such RNA transcripts of unknown sequence within ribonucleoprotein particles identify RNA sequences encoded by genes neighboring L1 elements and their protein products represent putative autoantigens.

### Detection of Serum Autoantibodies Specific for L1 Products

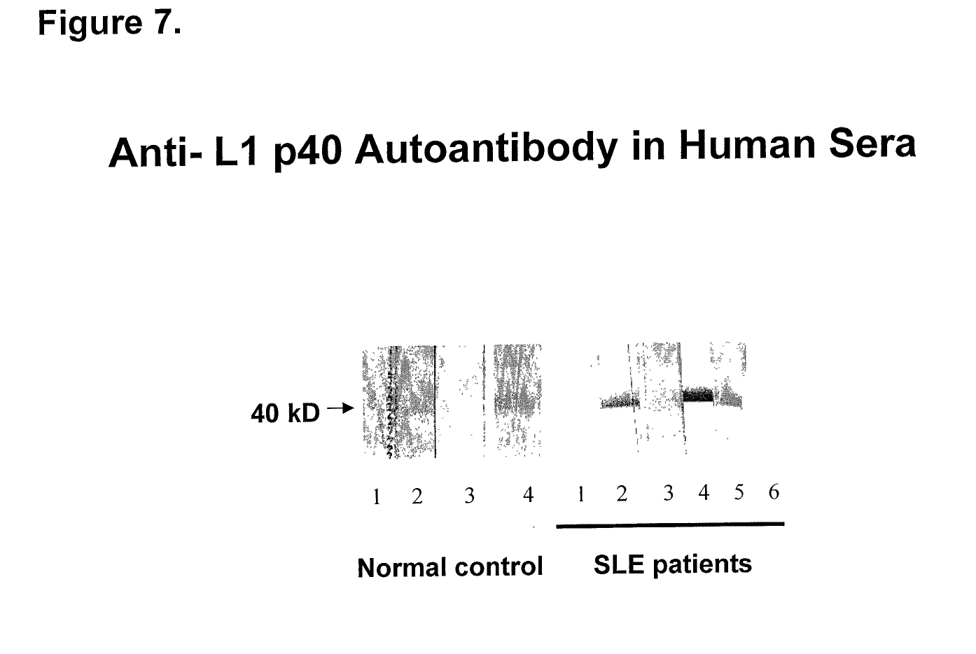

Patients with autoimmune disease, particularly those with SLE, often make antibodies with specificity for nucleotide or protein components of intracellular particles, including ribonucleoprotein particles. In accordance with the present invention, the presence of autoantibodies specific for L1 DNA or mRNA sequences or for L1 protein products indicating a diagnosis of autoimmune disease and detection of a change in the titer or level of those antibodies is associated with a change in the clinical disease activity of the patient. Serum autoantibodies specific for L1 products can be detected by the techniques of ELISA, with a recombinant form of the L1 protein product adsorbed to a plastic microwell and then reacted with patient or control serum, or by immunoblot, with patient serum used to detect the L1 DNA, RNA or protein products (FIG. 7) or by immunoprecipitation, in which the patient serum is used to precipitate cellular components containing L1 products, or purified L1 products.

### Disease-Specific Examples

The previous sections have described the general methodology for detecting disease genes, susceptibility to, or diagnosing, complex diseases via L1 element analysis. This section provides strategies for determining susceptibility to specific complex diseases such as autoimmune diseases. Examples include several organ specific autoimmune diseases, in which putative autoantigens can be localized in the genome; SLE, the prototype autoimmune disease; Alzheimer disease, a common dementia in which a region of chromosome 21 has been implicated; and schizophrenia, a common psychotic disease for which recent genome studies have identified genomic loci with statistically significant associations with disease.

### Pemphigus Foliaceus

In order to determine susceptibility to pemphigus foliaceus, the region of chromosome 18q12 encoding desmoglein 1 (sequence in contig NT_{—}010966) and an L1 element with 95% sequence homology to the consensus sequence in the 5′ region is characterized in DNA from study subjects using PCR amplification, a region-specific DNA probe, or by direct DNA sequencing. This L1 element is contained within the coding sequence of DSG1. The results of those assays are compared to results using DNA from control individuals. Expression of desmoglein 1 mRNA or protein in association with L1 mRNA or protein can also be assayed using tissue from skin biopsies. Elevated levels (as described above) of L1 mRNA or protein in serum, plasma, or urine also indicates susceptibility to or diagnosis of autoimmune disease, such as pemphigus.

### Autoimmune Thyroid Disease

In order to determine susceptibility to autoimmune thyroid disease, the region of chromosome 14q31 encoding thyroid stimulating hormone receptor that contains an L1 element with 94% sequence homology to the consensus sequence in the 5′ region contained within the coding region of TSHR on contig NT_{—}010140 is characterized in DNA from study subjects using PCR amplification, a region-specific DNA probe, or by direct DNA sequencing and the results of those assays compared to results using DNA from control individuals. Expression of thyroid stimulating hormone receptor mRNA or protein in association with L1 mRNA or protein can also be assayed using peripheral blood lymphocytes or tissue from thyroid biopsies. Elevated levels (as described above) of L1 mRNA or protein in serum, plasma, or urine also indicates susceptibility to or diagnosis of autoimmune disease, such as autoimmune thyroid disease.

### Primary Biliary Cirrhosis

In order to determine susceptibility to primary biliary cirrhosis, the region of chromosome 13q37 encoding the protein identified as similar to nuclear antigen SP100 protein (LOC93350) (sequence in contig NT_{—}026242) and the nearby L1 element with 95% sequence homology to the consensus sequence in the 5′ region contained within the coding sequence of LOC 93350 is characterized in DNA from study subjects using PCR amplification, a region-specific DNA probe, or by direct DNA sequencing and the results of those assays compared to results using DNA from control individuals. Expression of SP100 mRNA or protein in association with L1 mRNA or protein can also be assayed using peripheral blood lymphocytes or tissue from liver biopsies. Elevated levels (as described above) of L1 mRNA or protein in serum, plasma, or urine also indicates susceptibility to or diagnosis of autoimmune disease, such as primary biliary cirrhosis.

### Systemic Autoimmune Diseases

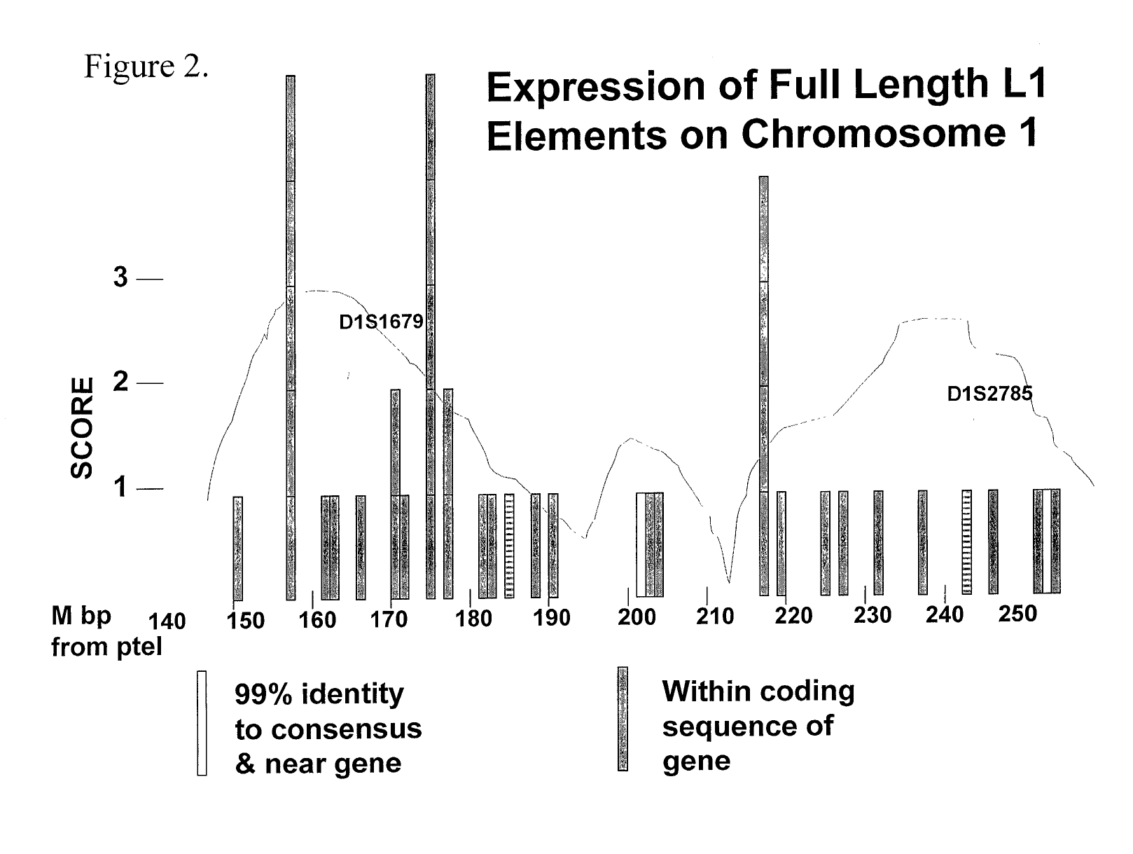

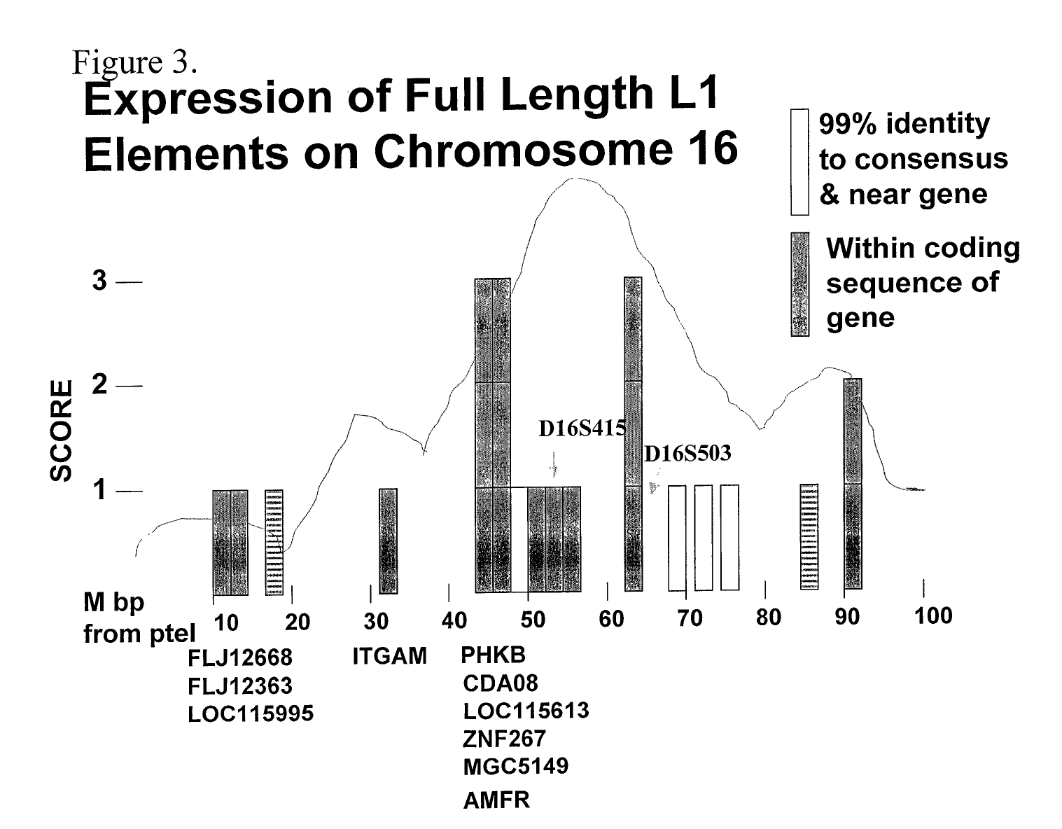

Systemic autoimmune diseases include, e.g., SLE, mixed connective tissue disease, scleroderma, and Sjögren's syndrome. These autoimmune diseases can be initiated by an immune response to cellular components containing products of L1 elements. The procedure to determine susceptibility to a systemic autoimmune disease is outlined above. Briefly, a map of the location of high fidelity intact L1 elements or full-length L1 elements located in coding regions of genes, or within 100,000 bases of the 5′ or 3′ extent of a gene, is generated, the DNA in those regions of the genome is characterized in subjects being studied for susceptibility to a systemic autoimmune disease, and the number and DNA sequences of those regions compared to healthy control subjects. Alternatively, investigation can be focused on the genomic loci identified in genome screens by microsatellite loci or single nucleotide polymorphism studies, with the full length L1 elements in the approximately 5 million bases on either side of the identified locus searched. The map of full length L1 elements within coding sequences of genes (FIG. 2) and high fidelity full length L1 elements within 100,000 bases of a gene on chromosome 1q serves as an example of the procedure, but all such L1 elements across the genome should be studied (as in Table 3 for all of chromosome 1q and in FIG. 3 for all of chromosome 16). On chromosome 1q, such L1 elements are found in: contig NT_{—}029226 (L1 of 89% identity to consensus sequence in coding sequence of CEZANNE at 150M from ptel); contig NT_{—}4858 (five L1 of 94, 87, 85, 84, and 84% identity to consensus sequence in coding sequence of LOC 128249 at 157.95M from ptel; L1 of 98% identity to consensus sequence within 100,000 bases of FLJ0024 at 167.4 from ptel; L1 of 94 and 91% identity to consensus sequence in coding sequence of NME7 at 170.2-170.5M bases from ptel; L1 of 89% identity to consensus in coding sequence of ATF6 at 162.3M bases from ptel; L1 of 87% identity to consensus in coding sequence of DDR2 at 163.8M bases from ptel; L1 of 88% identity to consensus in coding sequence of ALDH9A1 at 166.75M bases from ptel; and L1 of 81% identity to consensus in coding sequence of KIFAP3 at 171.08M bases from ptel); contig NT_{—}029874 (five L1 of 95, 93, 87, 81, and 79% identity to consensus in LOC127100 at 175.5M bases from ptel); contig NT_{—}029868 (L1 of 98 and 92% identity to consensus sequence in LOC127055 at 178.54M bases from ptel); contig NT_{—}026949 (L1 of 83% identity to consensus in FLJ10244 at 180.6M bases from ptel; L1 of 89% identity to consensus in NPHS2 at 181.8M bases from ptel); contig NT_{—}004552 (L1 of 99% identity to consensus in XPR1 at 184.28M bases from ptel); contig NT_{—}029219 (L1 of 98% identity to consensus within 100,000 bases of LOC126918 at 186.7M bases from ptel); contig NT_{—}004487 (L1 of 98% identity to consensus in coding sequence of C1ORF24, NIBAN, at 189M bases from ptel; L1 of 98% identity to consensus within 100,000 bases of LOC 127523 at 191.9M bases from ptel; L1 of 98% identity to consensus within 100,000 bases of LOC127522 and LOC127521 at 191.5M bases from ptel; L1 of 91% identity to consensus within coding sequence of FIBL-6 at 190.65M bases from ptel); contig Nt_{—}004671 (l1 OF 98% identity to consensus within coding sequence of LOC127964 at 198.58M bases from ptel); contig NT_{—}004416 (L1 of 99% identity to consensus within 100,000 bases of LOC127387 at 202.65M bases from ptel; L1 of 93% identity to consensus within coding sequence of LOC127388 at 202.5M bases from ptel); contig NT_{—}029862 (L1 with 98% identity to consensus witin 100,000 bases of LOC127012 at 204.1M bases from ptel; L1 of 88% identity to consensus in coding sequence of FHR5 at 203.22M bases from ptel; L1 of 96% identity to consensus in coding region of F13B at 203.26M bases from ptel); contig NT_{—}021877 (L1 of 98% identity to consensus in coding sequence of LOC126615 at 217M bases from ptel); contig NT_{—}030578 (four L1 with 90, 89, 88, and 83% identity to consensus in coding sequence of KCNH1 at 217.84-218.23M bases from ptel); contig NT_{—}004993 (L1 with 85% identity to consensus in coding sequence of FLJ10874 at 119.68M bases from ptel); contig NT_{—}004817 (L1 of 98% identity to consensus within 100,000 bases of LOC128150 and LOC 128149 at 224.6M bases from ptel; L1 of 88% identity to consensus within coding sequence of FLJ10252 at 225.3M bases from ptel); contig NT_{—}029871 (L1 with 96% identity to consensus in coding sequence of RAB3-GAP150 at 228.1M bases from ptel); contig NT_{—}004861 (L1 with 85% identity to consensus in coding sequence of FLJ10052 at 231.6M bases from ptel); contig NT_{—}004753 (L1 with 91% identity to consensus in coding sequence of DISC1 at 238.61 to 239.13M bases from ptel); contig NT_{—}004836 (L1 of 99% identity to consensus in coding sequence of RYR2 at 244.3M bases from ptel; L1 of 88% identity to consensus in coding sequence of TM7SF1 at 243.38M bases from ptel); contig NT_{—}004771 (L1 of 98% identity to consensus within 100,000 bases of LOC114922 at 248.38M bases from ptel; L1 of 91% identity to consensus in coding sequence of RGS7 at 247.52M bases from ptel); contig NT_{—}004734 (L1 of 86% identity to consensus in coding sequence of AKT3 at 251.3 to 251.4M bases from ptel); and contig NT_{—}004536 (L1 of 99% identity to consensus within 100,000 bases of LOC127615 and LOC127616 at 252.6M bases from ptel; L1 of 95% identity to consensus within coding sequence of FLJ21080 at 254.2M bases from ptel). These identified genes and predicted genes represent candidate disease genes from among the approximately 1600 genes and predicted genes on human chromosome 1q and as such, may warrant consideration for further study for involvement in the pathogenesis of autoimmune and other diseases, for involvement in a molecular pathway involved in the pathogenesis of autoimmune and other diseases, for susceptibility genes for these diseases, and as potential targets for therapy of such diseases. Similar analyses can be performed in any other region of the genome. Each of these chromosomal regions can be characterized in a study subject by PCR amplification, a region-specific DNA probe, or by direct DNA sequencing and the results of those assays compared to results using DNA from control individuals. The presence of an increased number of productive L1 sequences in an individual's genome or in the coding regions, particularly intronic regions, of genes would be associated with increased susceptibility to systemic autoimmune disease or other disease.

In addition to increased numbers of productive L1 elements, altered expression of a gene product implicated in immune system function, inflammation, or other pathway relevant to pathogenesis of autoimmune disease based on proximity of an L1 element to that gene may confer susceptibility to systemic autoimmune diseases. A map of genes may that are proximate to L1 elements can be constructed and the DNA sequences in those regions be determined by characterizing DNA from study subjects using PCR amplification, a region-specific DNA probe, or by direct DNA sequencing and the results of those assays compared to results using DNA from control individuals. For example, to determine susceptibility to SLE, the region of chromosome 1q encoding FcgRIIb (contig NT_{—}004668) and the nearby L1 element with 89% sequence homology to the consensus sequence in the 5′ region and contained within the coding sequence of ATF6, a cAMP dependent transcription factor, is characterized in DNA from study subjects using PCR amplification, a region-specific DNA probe, or by direct DNA sequencing and the results of those assays compared to results using DNA from control individuals. The presence of the L1 element in this region would predict susceptibility to SLE.

### Alzheimer Disease

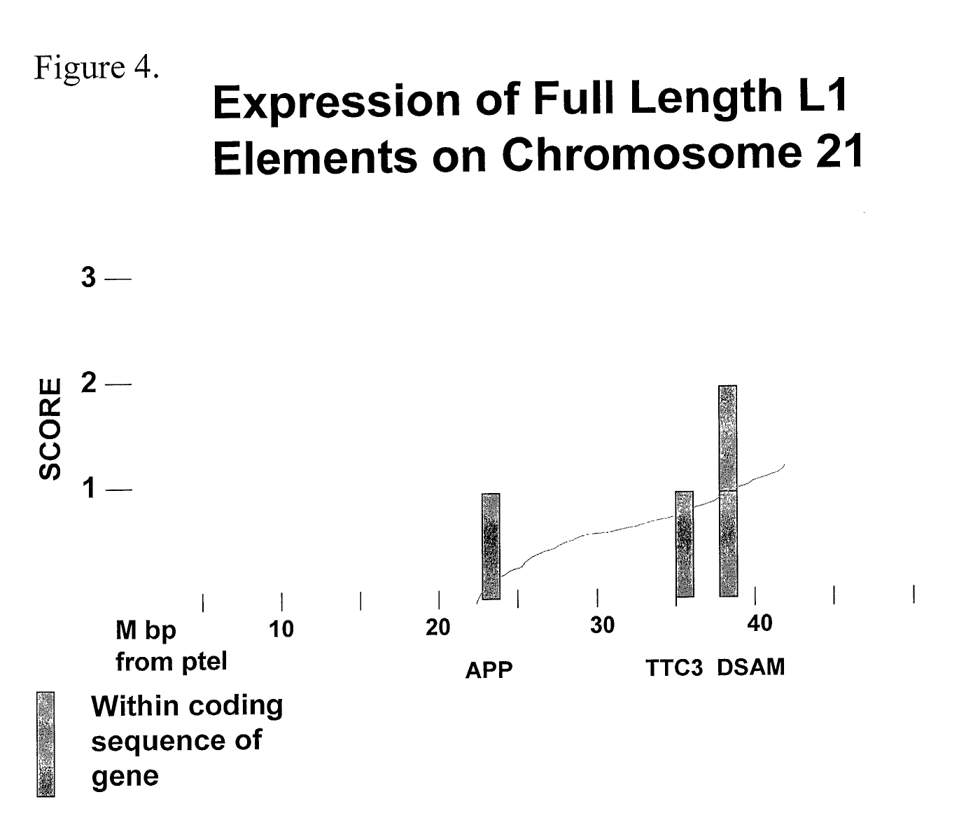

Identification of genes and genes products relevant to the pathogenesis of Alzheimer disease, and identification of individuals susceptible to developing the disease, can be determined by using a similar approach to that described for chromosome 1q, with the study directed toward those genomic regions that have been implicated in the disease. The entire genomic sequence of chromosome 21, which had been associated with a diagnosis of Alzheimer disease in family studies and sporadic cases, was analyzed for expression of full length and full length high fidelity L1 sequences within 100,000 bases of a gene or predicted gene. FIG. 4 shows the results of such analysis and demonstrates only three positive results: on contig NT_{—}011512 (L1 of 97% identity to consensus in coding sequence of APP at 23.0M bases from ptel; L1 of 90% identity to consensus in coding sequence of TTC3 at 35.1M bases from ptel; two L1 of 93 and 87% identity to consensus in coding sequence of DSCAM at 38.1M bases from ptel). APP encodes amyloid precursor protein, documented to be mutated in some familial cases of Alzheimer disease and proposed to be involved in a common pathogenic pathway in Alzheimer disease. The other two identified genes, are also excellent candidates for disease genes. TTC3 encodes a protein with a tetratricopeptide domain and DSCAM is Down's syndrome cell adhesion molecule.

### Schizophrenia

Identification of genes that may be important in the pathogenesis of schizophrenia are similarly identified by analysis of chromosomal regions adjacent to loci statistically associated with a diagnosis of schizophrenia. Table 2 lists some susceptibility loci and some candidate genes, based on analysis of L1 sequences. These loci and associate candidate genes include: DIS196 located 170.1M bases from ptel on chromosome 1 with associated L1-containing genes KIFAP1 (kinensin associated protein, expressed in cerebellum, at 171.08M bases from ptel) and DDR2 (neurotrophic receptor tyrosine kinase receptor related protein at 163.8M bases from ptel); D4S430 located 115.4M bases from ptel on chromosome 4 with associated gene CAMK2D (calcium calmodulin delta 2 kinase, expressed in hippocampal and pyramidal cells at 113.9M bases from ptel); D5S422 located 167.97 from ptel on chromosome 5 and associated gene GLRA1 (glycine receptor alpha 1, implicated in startle disease and stiff man syndrome, at 161.9M bases from ptel); D8S503 located 7.28M bases from ptel on chromosome 8 and associated genes DLGAP2 (concentrated in synaptic junctions and in hippocampus at 1.5M bases from ptel), CSMD1 (with domains abundant in complement control proteins at 3.8M bases from ptel), and FDFT1 (famesyltransferase, active in cholesteral biosynthesis, at 12.1M bases from ptel); D8S1771 located 26.48M bases from ptel on chromosome 8 and associated genes BNIP3L (a proapoptotic protein at 27.3M bases from ptel), LOC137822 (gene with protein of unknown function containing an L1 with 99% identity to the consensus in its coding region, at 27.9M bases from ptel), and WRN.RECQL2 (Werner's syndrome gene at 31M bases from ptel); D11S934 located 132.8M bases from ptel and associated genes TEKTA (encoding a protein associated with deafness at 129M bases from ptel) and GRIK4 (a glutamate receptor gene expressed in brain located at 129M bases from ptel); and D20S112 located at 17.25M bases from ptel with associated genes PGAM-B (similar to brain phosphoglycerate mutase at 9.55M bases from ptel) and LOC96688 (a neuroendocrine convertase 2 precursor at 17.16M bases from ptel). All of these genes containing full length L1 elements in their coding regions and are proposed as potential candidate disease genes in schizophrenia.

### Prevention and Treatment of Complex Diseases

According to the invention, inhibition of the expression or function of L1 mRNA or protein products can be used to prevent or treat autoimmune diseases. The expression of relatively increased cellular levels of mRNA transcripts of L1 elements, the protein products of ORF1 or ORF2, or L1 mRNA products in close association with mRNA or protein products of other host genes can confer an autoimmune or other pathogenic state on an individual. Therefore, decreasing the quantity or activity of such L1 products in order to inhibit or decrease the disease activity in an individual patient, or to prevent the initiation of autoimmune disease in a susceptible individual is a preferred embodiment of the present invention.

There are many standard or proposed approaches to inhibiting the expression or function of gene products, including both mRNA and protein products. These approaches can act on the conformation or biochemical composition of DNA or the proteins, such as histones, that associate with DNA. Promotion of DNA methylation, inhibition of DNA demethylation, promotion of histone deacetylation, and inhibition of histone acetylation are examples of such approaches. Transcription factors that bind to regulatory DNA elements can be specifically targeted to inhibit gene transcription. mRNA can either be specifically inhibited using agents such as anti-sense, or mRNA stability can be manipulated by augmenting or inhibiting proteins that bind to the specific mRNA and modify the degradation of that mRNA.

For example, hypermethylation mediated by proteins such as DNA-methyltransferase is associated with transcriptional inactivation in both normal cells and in some cancers (73, 74, 75). Demethylation with 5-aza can restore gene transcription (75). Alternatively, histone acetyltransferases contribute to relaxation of chromatin structure and gene transcription (76), and histone deacetylases can function as transcriptional repressors (77). Biochemical modifiers of this process include suberoylanilide hydroxamic acid, a histone deacetylase inhibitor, or trichostatin (78). Transcription factors that bind to regulatory DNA elements can be specifically targeted to inhibit gene transcription. The SRY protein is an example of a protein that inhibits transcription of L1 elements. mRNA can be specifically inhibited or degraded using agents such as anti-sense or mediators of RNA interference (79). mRNA stability can also be manipulated by augmenting or inihibiting proteins that bind to the specific mRNA and modify the degradation of that mRNA. For example, proteins that bind to the 3′ untranslated region of an mRNA and stabilize that mRNA, the suggested role of members of the HuR family of proteins, might be inhibited, or proteins that mediate mRNA degradation, such as tristetraprolin, might be induced (80, 81). It should be noted that the state of the art regarding regulation of mRNA stability does not at present define all proteins that regulate mRNA stability or their functions.

The protein products of L1 elements, the ORF1 and ORF2 proteins, can also be targeted for inhibition by antibodies, such as specific monoclonal antibodies, or small protein inhibitors that block the actions of those proteins. Therapeutic inhibition of the mRNA or protein products of L1 elements is expected to decrease the availability or activity of the immunologic stimulus for autoimmune disease, to improve the clinical activity of that disease, or to inhibit the initiation of the initial disease state. In one embodiment, monoclonal antibodies immunoreactive with the ORF1 and/or ORF2 proteins are generated using routine procedures well known to those of ordinary skill in the art.

### Monoclonal Antibodies Against ORF1 and/or ORF2

The general methodology for making monoclonal antibodies by hybridomas is well known. See, e.g., Kohler et al., 1980, Hybridoma Techniques, Cold Spring Harbor Laboratory, New York; Tijssen, 1985, Practice and Theory of Enzyme Immunoassays, Elsevier, Amsterdam; Campbell, 1984, Monoclonal Antibody Technology, Elsevier, Amsterdam; Hurrell, 1982, Monoclonal Hybridoma Antibodies: Techniques and Applications, CRC Press, Boca Raton, Fla. Purification methods for antibodies are disclosed, e.g., in The Art of Antibody Purification, 1989, Amicon Division, W.R. Grace & Co.

In a preferred embodiment, when the antibodies are used therapeutically to treat humans, the antibodies are “humanized”, i.e., human Fc sequences are present in the antibody molecule to prevent an adverse immune response in a patient to whom the antibodies are administered. When used to treat patients suffering from a complex disease as defined herein, such antibodies can be administered in amounts effective to treat or prevent the manifestation of the symptoms of these diseases. These effective amount broadly ranges between about 1 and 1000 mg per kg body weight of said mammal. The antibodies can be administered systemically, preferably parenterally and most preferably subcutaneously or intravenously.

### Other Ligands

The identification of L1 elements provides for development of screening assays, particularly for high throughput screening of molecules that modify, up- or down-regulate, i.e., inhibit or stimulate, agonize or antagonize, the transcription or translation activity of the L1 element. Alternatively, anti-sense oligonucleotides can be used to prevent L1 transcripts from translation, or to prevent L1 transcipts, ORF1, or ORF2 from associating to susceptibility genes, their corresponding mRNA or translation products. The present invention contemplates screens for small molecule ligands or ligand analogs and mimics, as well as screens for natural ligands to L1 molecules.

Any screening technique known in the art can be used to screen for compounds which up- or down-regulates the transcription or translation activity of the L1 element. For instance, a screening assay can be based on measurement of the amount or formation rate of transcribed L1 mRNA by a suitable method, or transcription of the L1 gene resulting in the formation or release of a reporter molecule which can be easily measured. Generally, a screening assay involves contacting the L1 gene, mRNA, or protein sequence with a compound which interacts or otherwise affects the conformation or activity of the sequence. The L1 promoter sequence can be linked to cDNA encoding for a reporter protein, or another polypeptide or protein. The transcriptional activity of the promoter is measured in the presence of the compound, and compared to a control value. This control value could be, for example, transcriptional activity of the promoter in the absence of the compound, transcriptional activity of the promoter in the presence of a reference compound with a known effect on transcriptional activity, or another theoretically or experimentally derived value.

### Gene Therapy to Modulate L1 Transcription or Biological Activity

A LINE gene such as L1, or alternatively a negative regulator of the L1 element such as an antisense nucleic acid, intracellular antibody (intrabody), can be introduced in vivo, ex vivo, or in vitro using a viral or a non-viral vector, e.g., as discussed above. Expression in targeted tissues can be effected by targeting the transgenic vector to specific cells, such as with a viral vector or a receptor ligand, or by using a tissue-specific promoter, or both. Targeted gene delivery is described in International Patent Publication WO 95/28494, published October 1995.

Preferably, for in vivo administration, an appropriate immunosuppressive treatment is employed in conjunction with the viral vector, e.g., adenovirus vector, to avoid immuno-deactivation of the viral vector and transfected cells. For example, immunosuppressive cytokines, such as interleukin-12 (IL-12), interferon-γ (IFN-γ), or anti-CD4 antibody, can be administered to block humoral or cellular immune responses to the viral vectors (see, e.g., Wilson, Nature Medicine, 1995). In that regard, it is advantageous to employ a viral vector that is engineered to express a minimal number of antigens.

Adenovirus Vectors.

Adenoviruses are eukaryotic DNA viruses that can be modified to efficiently deliver a nucleic acid of the invention to a variety of cell types in vivo, and has been used extensively in gene therapy protocols. Various serotypes of adenovirus exist. Of these serotypes, preference is given to using type 2 or type 5 human adenoviruses (Ad 2 or Ad 5) or adenoviruses of animal origin (see W094/26914). Those adenoviruses of animal origin which can be used within the scope of the present invention include adenoviruses of canine, bovine, murine (example: Mavl, Beard et al., Virology 75 (1990) 81), ovine, porcine, avian, and simian (example: SAV) origin. Preferably, the adenovirus of animal origin is a canine adenovirus, more preferably a CAV2 adenovirus (e.g., Manhattan or A26/61 strain (ATCC VR-800), for example). Various replication defective adenovirus and minimum adenovirus vectors have been described for gene therapy (WO94/26914, WO95/02697, WO94/28938, WO94/28152, WO94/12649, WO95/02697 WO96/22378). The replication defective recombinant adenoviruses according to the invention can be prepared by any technique known to the person skilled in the art (Levrero et al., Gene 101:195 1991; EP 185 573; Graham, EMBO J. 3:2917, 1984; Graham et al., J. Gen. Virol. 36:59 1977). Recombinant adenoviruses are recovered and purified using standard molecular biological techniques, which are well known to one of ordinary skill in the art.

Adeno-Associated Viruses.

The adeno-associated viruses (AAV) are DNA viruses of relatively small size which can integrate, in a stable and site-specific manner, into the genome of the cells which they infect. They are able to infect a wide spectrum of cells without inducing any effects on cellular growth, morphology or differentiation, and they do not appear to be involved in human pathologies. The AAV genome has been cloned, sequenced and characterized. The use of vectors derived from the AAVs for transferring genes in vitro and in vivo has been described (see WO 91/18088; WO 93/09239; U.S. Pat. Nos. 4,797,368, 5,139,941, EP 488 528). The replication defective recombinant AAVs according to the invention can be prepared by co-transfecting a plasmid containing the nucleic acid sequence of interest flanked by two AAV inverted terminal repeat (ITR) regions, and a plasmid carrying the AAV encapsidation genes (rep and cap genes), into a cell line which is infected with a human helper virus (for example an adenovirus). The AAV recombinants which are produced are then purified by standard techniques.

Retrovirus Vectors.

In another embodiment the gene can be introduced in a retroviral vector, e.g., as described in Anderson et al., U.S. Pat. No. 5,399,346; Mann et al., 1983, Cell 33:153; Temin et al., U.S. Pat. No. 4,650,764; Temin et al., U.S. Pat. No. 4,980,289; Markowitz et al., 1988, J. Virol. 62:1120; Temin et al., U.S. Pat. No. 5,124,263; EP 453242, EP178220; Bernstein et al. Genet. Eng. 7 (1985) 235; McCormick, BioTechnology 3 (1985) 689; International Patent Publication No. WO 95/07358, published Mar. 16, 1995, by Dougherty et al.; and Kuo et al., 1993, Blood 82:845. The retroviruses are integrating viruses which infect dividing cells. The retrovirus genome includes two LTRs, an encapsidation sequence and three coding regions (gag, pol and env). In recombinant retroviral vectors, the gag, pol and env genes are generally deleted, in whole or in part, and replaced with a heterologous nucleic acid sequence of interest. These vectors can be constructed from different types of retrovirus, such as MoMuLV (“murine Moloney leukaemia virus”), MSV (“murine Moloney sarcoma virus”), HaSV (“Harvey sarcoma virus”); SNV (“spleen necrosis virus”); RSV (“Rous sarcoma virus”) and Friend virus. Suitable packaging cell lines have been described in the prior art, in particular the cell line PA317 (U.S. Pat. No. 4,861,719); the PsiCRIP cell line (WO 90/02806) and the GP+envAm-12 cell line (WO 89/07150). In addition, the recombinant retroviral vectors can contain modifications within the LTRs for suppressing transcriptional activity as well as extensive encapsidation sequences which may include a part of the gag gene (Bender et al., J. Virol. 61:1639, 1987). Recombinant retroviral vectors are purified by standard techniques known to those having ordinary skill in the art.

Retrovirus vectors can also be introduced by recombinant DNA viruses, which permits one cycle of retroviral replication and amplifies transfection efficiency (see WO 95/22617, WO 95/26411, WO 96/39036, WO 97/19182).

Lentivirus Vectors.

In another embodiment, lentiviral vectors are can be used as agents for the direct delivery and sustained expression of a transgene in several tissue types, including brain, retina, muscle, liver and blood. The vectors can efficiently transduce dividing and nondividing cells in these tissues, and maintain long-term expression of the gene of interest. For a review, see, Naldini, Curr. Opin. Biotechnol., 9:457-63, 1998; see also Zufferey, et al, J. Virol., 72:9873-80, 1998). Lentiviral packaging cell lines are available and known generally in the art. They facilitate the production of high-titer lentivirus vectors for gene therapy. An example is a tetracycline-inducible VSV-G pseudotyped lentivirus packaging cell line which can generate virus particles at titers greater than 106 IU/ml for at least 3 to 4 days (Kafri, et al., J. Virol., 73: 576-584, 1999). The vector produced by the inducible cell line can be concentrated as needed for efficiently transducing nondividing cells in vitro and in vivo.

Non-Viral Vectors.

A vector can be introduced in vivo in a non-viral vector, e.g., by lipofection, with other transfection facilitating agents (peptides, polymers, etc.), or as naked DNA. Synthetic cationic lipids can be used to prepare liposomes for in vivo transfection, with targeting in some instances (Feigner, et. al., Proc. Natl. Acad. Sci. U.S.A. 84:7413-7417, 1987; Feigner and Ringold, Science 337:387-388, 1989; see Mackey, et al., Proc. Nati. Acad. Sci. U.S.A. 85:8027-8031, 1988; Ulmer et al., Science 259:1745-1748, 1993). Useful lipid compounds and compositions for transfer of nucleic acids are described in International Patent Publications WO95/18863 and WO96/17823, and in U.S. Pat. No. 5,459,127. Other molecules are also useful for facilitating transfection of a nucleic acid in vivo, such as a cationic oligopeptide (e.g., International Patent Publication WO95/21931), peptides derived from DNA binding proteins (e.g., International Patent Publication WO96/25508), or a cationic polymer (e.g. , International Patent Publication WO95/21931). Recently, a relatively low voltage, high efficiency in vivo DNA transfer technique, termed electrotransfer, has been described (Mir et al., C.P. Acad. Sci., 321:893, 1998; WO 99/01157; WO 99/01158; WO 99/01175). DNA vectors for gene therapy can be introduced into the desired host cells by methods known in the art, e.g., electroporation, microinjection, cell fusion, DEAE dextran, calcium phosphate precipitation, use of a gene gun (ballistic transfection), or use of a DNA vector transporter (see, e.g., Wu et al., J. Biol. Chem. 267:963-967, 1992; Wu and Wu, J. Biol. Chem. 263:14621-14624, 1988; Hartmut et al., Canadian Patent Application No. 2,012,311, filed Mar. 15, 1990; Williams et al., Proc. Natl. Acad. Sci. USA 88:2726-2730, 1991). Receptor-mediated DNA delivery approaches can also be used (Curiel et al., Hum. Gene Ther. 3:147-154, 1992; Wu and Wu, J. Biol. Chem. 262:4429-4432, 1987). U.S. Pat. Nos. 5,580,859 and 5,589,466 disclose delivery of exogenous DNA sequences, free of transfection facilitating agents, in a mammal.

The knowledge derived from the procedures described above would allow for better diagnostic procedures for identifying individuals at risk for, susceptible to, or predisposed to complex diseases in which an L1 element is a direct or indirect factor. The correlation between the distance of an L1 element from, or the presence of an L1 element in an intron sequence of, a susceptibility gene, to disease susceptibility and progression, will provide for a better understanding of the causes and progression of autoimmune and other complex diseases, as well as novel therapeutic strategies for treating such diseases.

### EXAMPLES

The present invention will be better understood by reference to the following Examples, which are provided as exemplary of the invention, and not by way of limitation.

### Example 1

### Identification of High Fidelity L1 Elements along Chromosome 1q

Chromosome 1q BAC clones, or contig clones (combining sequences from several BAC clones placed in proper order), were identified and ordered based on the contigs or BACs listed in the NCBI database, along with BACs or contigs identified by BLAST searching chromosome 1q microsatellite markers against the non-redundant and hgts human sequence databases.

Using the BLAST program for comparison of two sequences, all 80contigs on chromosome 1q, containing about 1600 genes, were compared to the 5′ and ORF1 sequence of LRE2, the L1 element previously localized to chromosome 1q and derived from a mutagenic insertion into a dystrophin gene (accession U09116). Of those clones, some were found to contain at least partial 5′ L1 sequences, while most included 3′ fragments. 26 genes were found to contain full length L1 sequences in their coding regions and some additional L1 sequences were found in close proximity (within 100,000bases) of a gene or predicted gene (see Table 3). These 26 genes were chosen for further study.

### Example 2

### Relationship of High Fidelity L1 Elements to SLE Susceptibility Loci

To relate the identified full-length high fidelity L1 sequences to previously identified SLE susceptibility loci, the data from several genome screens using microsatellite markers were relied upon (3, 4, 91). With increased availability of chromosome 1q BAC sequences, the precise location of the various microsatellite markers can be tied to specific BAC clones and localized along the chromosome more accurately than previously, although the location of some markers defined by radiation hybrid analysis remains ambiguous. Markers demonstrated by several investigators to characterize susceptibility loci were located using the chromosome mapping database available through NCBI.

Of 10 chromosome markers localized, 6 were within 1.7 cM of a potentially active L1 element. The 3 other loci, including the FCGR2A and MHC loci, may be associated with SLE through a mechanism that does not involve L1 elements. Alternatively, the disease marker may reflect the proximity of a gene in which a full-length, but not 98% or 99% identical to consensus, L1 element is included within the intronic region of a nearby gene. This is the case for FCGR2A, with an 89% identical to consensus L1 element in the intron of ATF6, immediately adjacent to FCGR2A, and with an 87% identical to consensus element in an intronic region of DDR2, approximately 1.2M bases from FCGR2A. The same may be true of D6S2410, which has LOC94915, a gene with possible calmodulin like calcium binding domains, at 1.63M bases from the marker and having an intronic L1 with 86% identity to the consensus sequence.

A more thorough analysis of chromosome 16 has identified additional candidate disease genes as indicated in FIG. 3 and Table 4. Notable candidate genes include ITGAM at 32M bases from ptel and with an 88% identical to consensus L1 in an intron; PHKB at 47.7M bases from ptel and with 3 L1 elements with 90%, 85%, and 82% sequence identity to the consensus; cadherin 8, at 64.3M bases from ptel with 3 L1 elements with 97%, 96%, and 95% sequence identity to consensus; and CDH13, a cadherin expressed in heart, at 87.1M bases from ptel with a 99% identical to consensus L1 element in an intronic region.

### Example 3

### Variability in 5′ Regulatory Region of L1 Elements

The data in the previous Examples showed an association between the location of high fidelity L1 elements and SLE susceptibility loci. To address the basis of this variability that may be linked to microsatellite markers in individuals, the literature was first considered and a comparison initiated of replicate copies of a single L1 element available in the database. Kazazian's group had documented polymorphism in expression of particular active L1 elements, as well as sequence variability in those that are expressed (43). For example, the gene frequency of LRE2 in the diploid genome was estimated at 0.65 and that of L1.3, on chromosome 14, of only 0.15. Thus, disease susceptibility is increased by the presence of an active element in an individual susceptible to SLE and relative protection is conferred by the absence of the active L1 in an individual.

An initial effort to study sequences more 5′ to the published consensus 5′ sequence used a several hundred bp sequence 5′ of a high fidelity L1 element located on chromosome 1q (AL162431, with 99% homology to the 5′ of U09116). This L1 may represent the genomic equivalent of LRE2 and is particularly intriguing as it is adjacent to the gene for a cell surface retroviral receptor. This 5′ region identified numerous BAC clones with high fidelity L1 elements.

### Example 4

### Identification of L1 Elements Adjacent to Disease-Relevant Genes

While the previous Examples localized predicted L1 sequences with the capacity to produce full length coding region RNAs, as well as ORF1, and possibly ORF2, proteins, it is the particles containing those components that would be immunogenic. However, one of the chromosome 1q loci that has received the most attention and some strong support for linkage to SLE, containing the Fcg receptor genes, did not show a nearby high fidelity L1 element (the calculated distance for FCGR2A was 4.78M bp). In view of the variable expression of some L1 elements among individuals, it is possible that the BAC clones from the FcR region published in the database does not include such a sequence, yet a clone from another individual over the same interval might. Eight overlapping clones were present at 162.3M bases from ptel of 1q. When compared to the 5′ and ORF1 L1 consensus sequence, no significant similarity was detected by the BLAST program for 5 of the BAC clones while one clone (AL391825) contained a full length L1 element with 89% identity to the consensus. Two other clones had a partial L1 sequence (AC027205 and AL359541). This locus may reflect the polymorphic expression of an L1 element in some individuals but not others.

The presence of a low fidelity L1 sequence within or near a disease-relevant gene raised the possibility that the third or fifth proposed mechanism for induction of human disease by L1 elements might pertain to SLE. Gene expression, function, or immunogenicity might be modulated by virtue of the presence of the regulatory L1 sequences or by coordinated transcription of both L1 and adjacent genes. To begin to investigate a potential disease-related role for L1 elements in genes that have been studied in conjunction with organ-targeted immune responses, the thyroid stimulating hormone receptor gene on chromosome 14q31, 79.6M bases from ptel, was analyzed. Contig NT_{—}010140 contained a full length L1 element with 94% identity to the consensus L1 sequence within an intronic region. Similarly, the DSG1 gene, an autoantigen for pemphigus foliaceous, on chromosome 18q12, contains a full length L1 element of 95% sequence identity to the consensus in an intronic region. The expression of L1 sequences within intronic segments of a gene may confer increased immunogenicity on that gene product.

### Example 5

### Expression of Orf1 mRNA and Protein

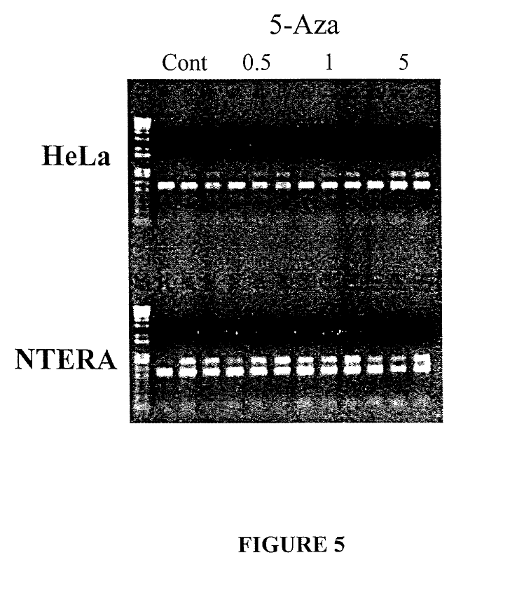

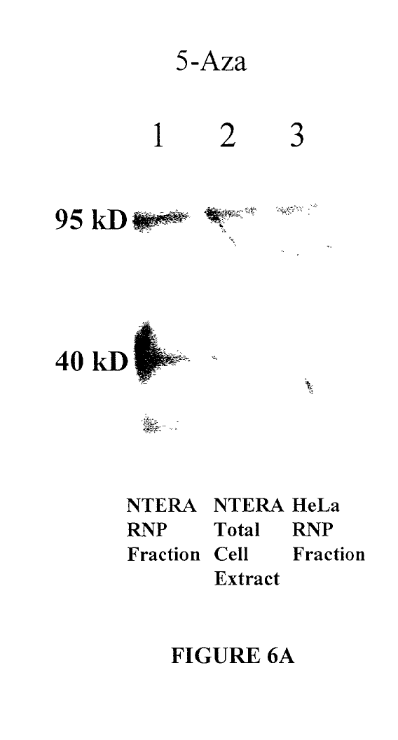

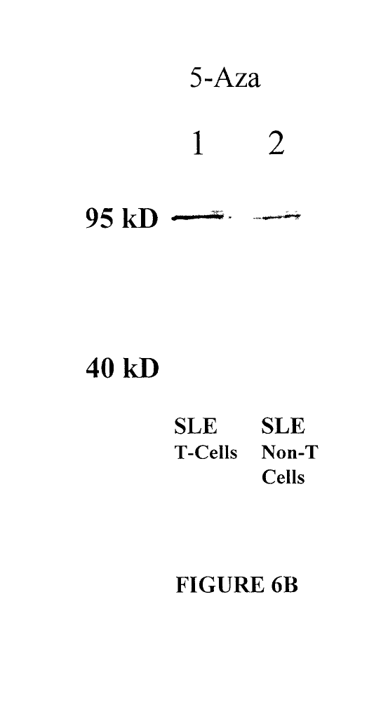

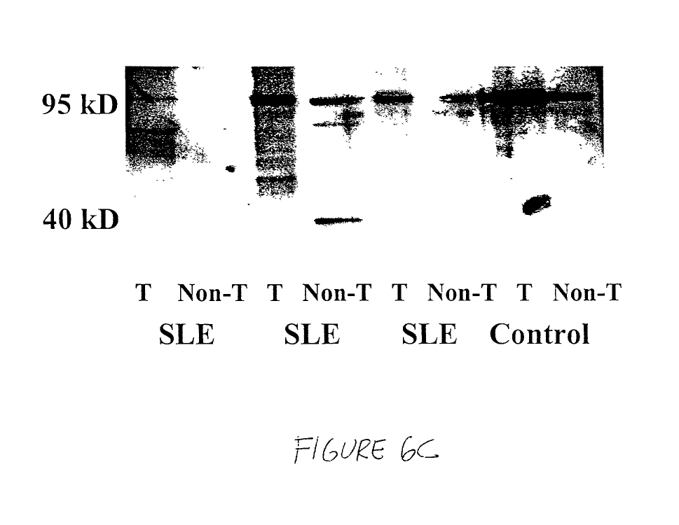

Enrichment of a genome in transcriptionally competent L1 elements would be predicted to result in detectable expression of L1 mRNA, and might also contribute to production of p40 and reverse transcriptase proteins. Cellular expression of ORF1p40 is seen in several teratocarcinoma cell lines, including NTERA-D1 (54). Several hints in the literature also suggest that some lymphocyte cell lines might express L1 p40 (55). Consistent with possible production of this protein in lymphocytes, it has been suggested that L1 products might serve an important cellular function in the repair of double stranded breaks, as occur in the setting of VDJ recombination or immunoglobulin class switching (60). For study of L1 product expression in SLE, three assays have been established, competitive mimic PCR to detect ORF1 p40 mRNA, real time PCR to detect ORF1 p40 mRNA, and Western blot to detect p40 protein. Total cellular RNA was isolated from NTERA teratocarcinoma cells or from HeLa cells, treated with DNAse followed by column purification to remove genomic DNA, reverse transcribed into cDNA, and then amplifed by PCR in the presence of 0.2-20 attomoles of a mimic construct containing a segment of the ORF1 coding sequence. In this assay, the relative concentration of target (cellular ORF1 cDNA) can be determined by noting the mimic concentration which is outcompeted by target (FIG. 5). While HeLa cells showed only trace concentrations of ORF1 cDNA, NTERA ORF 1 was readily detected. CpG motifs are present in the 5′ regulatory region of L1, and it has been proposed that demethylation might contribute to transcriptional activation. To determine if demethylation modulates ORF1 mRNA expression by HeLa or NTERA cells, the cells were treated with 0.5, 1.0, or 4 mM 5-azacytidine (5-Aza), known to result in demethylation of CpG dinucleotides. 4 mM 5-Aza induced a modest increase in ORF 1 mRNA in HeLa cells, while the already evident mRNA in NTERA cells was increased by even 0.5 mM 5-Aza. It is interesting to note that some lupus-inducing drugs mediate DNA demethylation, and 5-Aza can induce self-reactive T cells and lupus-like disease in an animal model (66). Induction of L1 gene activation might be a mechanism that accounts for these effects of DNA demethylation. Studies have also been initiated to study expression of ORF1 mRNA in lymphoid cell lines. Product has been detected in the D1.1 Jurkat cell variant and the CL-01 Burkitt's lymphoma B cell line in preliminary experiments.

As noted, readthrough transcripts of cellular genes that also contain fragments of L1 sequence are ubiquitous. While most of those background sequences are derived from ORF2, while we are amplifying ORF 1, all future experiments will be performed using polyA RNA rather than total cellular RNA, to enrich for those mRNAs specific to ORF 1.

To detect ORF1 protein, a Western blot was established which uses a rabbit antibody specific for ORF1 and is preadsorbed to remove nonspecific reactivities (54). Immunoblot analysis of protein extracts from HeLa and NTERA cells showed several nonspecific high molecular weight bands, also reported in the literature, along with a strong 40 kD band in NTERA (FIG. 6.A). A weak 40 kD band was also observed in HeLa cells in some experiments. As functional ORF1 p40 protein has been shown to be enriched in cytoplasmic RNP particles, that fraction was isolated by ultracentrifugation. In some experiments, the purification step resulted in a marked enrichment in the p40 protein band, while in others that fraction showed some additional degradation products. The RNP particle fraction can be used to increase the sensitivity of detection of the p40 protein in future experiments.

### Example 6

### Analysis of ORF1 Protein in SLE Lymphocytes

Increased expression of ORF 1 mRNA and protein would reflect either increased number and/or transcriptional activity of the complement of intact L1 elements in an individual's genome. It is therefore possible to detect those products. An important issue, however, is the choice of cell population for detection of ORF1 products. Ovary is predicted to be most enriched in ORF1 protein in females, but this tissue is rarely accessible. Based on the preliminary data showing ORF1 mRNA in several lymphoid cells lines, as well as some similar data in the literature, it was considered that lymphoid cells might on occasion express L1 protein (55). The speculation by others that L1 products might assist in the DNA repair process during events such as immunoglobulin class switching was intriguing in view of the augmented class switching in SLE that contributes to generation of pathogenic IgG autoantibodies.

Peripheral blood T and non-T cell fractions were isolated from 4 SLE patients and several healthy individuals, protein extracts subjected to ultracentrifugation to enrich for RNP particles, and that fraction analyzed by western blot. In these preliminary experiments, while no p40 bands have been observed in samples from controls, one of the four SLE non-T cell preparations showed a clear 40 kD protein detected with the anti-ORF1 antiserum (FIG. 6B). T cells from that individual were negative.

In another experiment (FIG. 6.C), non-T cells from all three SLE patients studied showed a band of approximately 40 kD after immunoblotting with the anti-p40 antibody, while non-T cells from a healthy control, and the T cell fractions from all subjects, were negative for a 40 kD band

### Example 7

### Production of L1 ORF1 mRNA and Protein in SLE Lymphocytes

In view of the intriguing finding of p40 protein in the non-T cell fraction of 1 of 4 SLE patients studied, p40 mRNA and protein in T and non-T cell fractions from normal peripheral blood and human tonsil cells are compared. Normal peripheral blood T and B cells can be negative, while tonsil B cells can give some signal. The tonsil cells are fractionated into those with GC phenotype based on expression of typical cell surface markers to define the cell subset producing L1 products. ORF1 mRNA and p40 protein expression in SLE peripheral blood T and non-T cells is explored, as well as in RA and healthy controls. The presented model requires production of both L1 RNA and protein at some cellular site in SLE. Studies are therefore designed to investigate whether these L1 products are produced throughout the course of disease, or only during the initiation phase.

Individuals recently diagnosed with SLE and followed in the SLE Pediatric Rheumatology Clinic at HSS where active SLE patients between the ages of 14 and 20 are seen regularly are initially studied. A Pediatric SLE DNA Repository containing family member DNA is available for correlative DNA analyses. Initially, 20 patients with recent onset SLE, 20 RA patients, and 20 healthy controls are studied for expression of ORF1 mRNA and protein as described. Some samples undergo enrichment for the RNP fraction by ultracentrifugation prior to Western blot analysis. In additional experiments, cell fractions will be preincubated for 48 hours with 5-Aza, in the presence or absence of physiologically relevant stimuli (anti-CD3, commercially available from ATCC, Menassus, Va. +anti-CD28 mAbs or F(ab′)2 anti-IgM+recombinant human CD40 ligand), prior to RNA or protein extraction.

### Example 8

### Detection of Autoantibodies to L1 ORF1 p40 protein

Patients with systemic autoimmune disease produce antibodies to nucleic acids and their associated proteins contained within intracellular particles. Support for a role for L1 elements and their products in the induction of autoimmune disease would be provided by documentation of autoantibodies specific for L1 encoded proteins. A recombinant fusion protein comprising p40 protein tagged with 6 histidines was produced and used to study SLE and healthy control sera for the presence of anti-p40 autoantibodies by electrophoresing the recombinant p40 protein on a gel and performing a western blot procedure with sera. A band representing reactivity of immunoglobulin with the p40 protein was detected in sera from SLE patients and a serum sample from an MRL/lpr lupus mouse, but not in several normal sera and only very weakly in another normal serum sample (FIG. 7).

### Search for Genes and Gene Products Involved in Alzheimer Disease

Studies of extended families with early-onset Alzheimer disease provided support for an association of individual variability on chromosome 21 with that disease. To define the location of full-length high fidelity L1 elements in proximity to genes and the location of L1 elements included in intronic or untranslated regions of genes, the entire published sequence of chromosome 21 was searched. First, the public genome database available through NCBI was accessed and a list of all of the available contigs that covered that chromosome generated. In the case of chromosome 21, the majority of the sequence is included in a single contig with accession number NT_{—}011512. That large sequence was directly searched, and in addition, a list of the BAC clones that comprise the contig was generated in order to sequentially search the genome seqments that make up the larger contig. The search could also have been focused on the region of the chromosome neighboring published microsatellite markers associated with the disease.

A publicly available search program, BLAST 2 sequences, was used to compare each contig or BAC clone sequence to the most 5′ approximately 900 bases of the DNA sequence of U09116 (LRE2). The search revealed no full length high fidelity (98-100% identity to the 5′ L1 sequence) L1 elements in the whole of chromosome 21. However, the search did reveal three genes with full-length L1 elements in intronic gene segments: 1) APP, with an L1 element of 97% identity to the consensus sequence; 2) TTC3, with an L1 element of 90% identity to the 5′ L1 sequence; and 3) DSCAM, which includes two intronic L1 sequences, one with 93% and one with 87% identity to the 5′ L1 sequence (FIG. 4 and Table 5). Thus this search successfully identified APP, encoding amyloid precursor protein, as a candidate disease gene. Abundant data has linked the APP gene or altered regulation of the gene or protein product in Alzheimer disease. The search also identified two additional potential disease genes that might be relevant to other disease situations. While little information is available regarding TTC3, DSCAM is the gene encoding Down's syndrome cell adhesion molecule, a protein that has been implicated in Down's syndrome.

### Example 9

### Search for Genes and Gene Products Involved in Schizophrenia

Another example of the method of the invention begins with five chromosome loci defined by microsatellite markers identified in a screen of thirteen large families with schizophrenia (84). For each of the five markers, their location in a particular contig was identified by searching the NCBI nucleotide database against the microsatellite marker. For each marker, as list of contigs approximately 5 million bases on either side of the marker was generated. Each of the contigs was then searched against the most 5′ approximately 900 bases of the consensus L1 sequence U09116. The five lists of contigs, and the results of the search, are shown in Table 2.

Candidate disease-related genes could be identified for further testing. Of these, several appear to be particularly attractive candidates for involvement in a disease of the central nervous system, such as schizophrenia. This example is highly applicable to developing a series of candidate disease genes for any disease in which preliminary studies have generated credible susceptibility loci.

### Example 11

### Search for Genes and Gene Products Involved in SLE

This Example demonstrates a similar approach for a disease in which many loci with borderline statistical significance have been proposed to possibly identify disease genes. Total genome screens using microsatellite analysis of DNA from patients with SLE and their family members have been published. One of these studies was used to guide a study of several chromosomes rich in peaks with increased LOD score for linkage with SLE (4). Chromosome 1q had numerous peaks of increased LOD score; chromosome 16 had one major broad peak of increased LOD score; and chromosome 21 had a region of modestly increased LOD score. For each of these chromosomes, all contigs were listed and searched against the 5′ most 900 bases of U09116. Full-length high fidelity L1 sequences within 100,000 bases of a known or predicted gene and full-length L1 sequences included within introns or untranslated regions of known or predicted genes were identified.

The location of these elements were then displayed based on their location on the chromosome, with multiple L1 elements within a single gene identified by stacked bars (FIGS. 2, 3, and 4. The curves generated from the LOD scores of microsatellite markers studied in the Gaffney SLE study (4) were then freely drawn over the display of the identified L1 elements along chromosomes 1q, 16, and 21. Strikingly, the LOD curves closely follow the location of full-length high fidelity L1 elements and L1 elements within genes. These genes and their mRNA and protein products become candidates for further study of their disease relevance. In addition, the high fidelity L1 elements themselves may represent disease susceptibility genes, with their products contributing to the immune system activation characteristic of SLE.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims. It is further to be understood that values are approximate, and are provided for description.

### BIBLIOGRAPHY

1. Todd J A. Genetic analysis of type I diabetes using whole genome approaches. Proc Natl Acad Sci 1995;92:8560-8565.

2. Tsao B P, Cantor R M, Kalunian K C, et al. Evidence for linkage of a candidate chromosome 1 region to human systemic lupus erythematosus. J Clin Invest 1997;99:725-731.

3. Moser K L, Neas B R, Salmon J E, et al. Genome scan of human systemic lupus erythematosus: evidence for linkage on chromosome 1q in african-American pedigrees. Proc Natl Acad Sci 1998;95:14869-14874.

4. Gaffney P M, Kearns G M, Shark K B, et al. A genome-wide search for susceptibility genes in human systemic lupus erythematosus sib-pair famililes. Proc Natl Acad Sci 1998;95:14875-14879.

5. Harley J B, Moser K L, Gaffney P M, Behrens T W. The genetics of human systemic lupus erythematosus. Curr Opin Immunol 1998;10:690-696.

6. Concannon P, Gogolin-Ewens K J, Hinds D A, et al. A second-generation screen of the human genome for susceptibility to insulin-dependent diabetes mellitus. Nature Genetics 1998;19:292-296.

7. Mein C A, Esposito L, Dunn M G, et al. A search for type I diabetes susceptibility genes in families from the United Kingdom. Nature Genetics 1998;19:297-300.

8. Cornelis F, Faure S, Martinez M, et al. New susceptibility locus for rheumatoid arthritis suggested by a genome-wide linkage study. Proc Natl Acad Sci 1998;95:10746-10750.

9. Becker K G, Simon R M, Bailey-Wilson J E et al. Clustering of non-major histocompatibility comlex susceptibility candidate loci in human autoimmune diseases. Proc Natl Acad Sci 1998;95:9979-9984.

10. Tsao B P, Cantor R M, Grossman J M, et al. PARP alleles within the linked chromosomal region are associated with systemic lupus erythematosus. J Clin Invest 1999;103:1135-1140.

11. Shai R, Quismoria F P, L1 L, et al. Genome-wide screen for systemic lupus erythematosus susceptibility genes in multiplex families. Human Mol Genet 1999; 8:639-641.

12. Risch N J Searching for genetic determinants in the new millenium. Nature 2000;405:847-856.

13. Morel L, Rudofsky U H, Longmate J A, Schiffenbauer J, Wakeland E K. Polygenic control of susceptibility to murine systemic lupus erythematosus. Immunity 1994;1:219-229.

14. Salmon J E, Millard S, Schachter L A, et al. Fc gamma RIIA alleles are heritable risk factors for lupus nephritis in African Americans. J Clin Invest 1996;97:1348-1354.

15. Lernmark A, Ott J. Sometimes it's hot, sometimes it's not. Nature Genetics 1998;19:213-214.

16. Yoshiki T, Mellors R C, Strand M, August J T. The viral envelope glycoprotein of murine leukemia virus and the pathogenesis of immune complex glomerulonephritis of New Zealand mice. J Exp Med 1974; 140:101 1-1027.

17. Choi Y, Kappler J W, Marrack P, A superantigen encoded in the open reading frame of the 3′ long terminal repeat of mouse mammary tumor virus. Nature 1991;350:203.

18. Dyson P J, Knight A M, Fairchild S, Simpson E, Tomonari K, Genes encoding ligands for deletion of Vb11 T cells cosegregate with mammary tumor virus genomes. Nature 1991;349:531-532.

19. Frankel W N, Rudy C, Coffin J M, Huber B T, Linkage of Mls genes to endogenous mammary tumor viruses of inbred mice. Nature 1991;349:526.

20. Woodland D L, Happ M P, Gollub K J, Palmer E, An endogenous retrovirus mediating deletion of abT cells? Nature 1991;349:529-530.

21. Woodland D L, Lund F E, Happ M P, Blackman M A, Palmer E, Corley R B, Endogenous superantigen expression is controlled by mouse mammary tumor proviral loci. J Exp Med 1991;174:1255-1258.

22. Beutner U, Frankel W N, Cote M S, Coffin J M, Huber B T, Mls-1 is encoded by the long terminal repeat open reading frame of the mouse mammary tumor virus Mtv-7. Proc Natl Acad Sci USA 1992;89:5432-5436.

23. Pullen A M, Choi Y, Kushnir E, Kappler J, Marrack P, The open reading frames in the 3′ long terminal repeats of several mouse mammary tumor virus integrants encode Vb3-specific superantigens. J Exp Med 1992;175:41-47.

24. Acha-Orbea H, Held W, Waanders G A, et al. Exogenous and endogenous mouse mammary tumor virus superantigens. Immunol Rev 1993;131:5-25.

25. Ross S R, Immunobiology of MMTV superantigens. In: Leung DYM, Huber B T, Schlievert P M, Eds. Superantigens. Molecular Biology, Immunology, and Relevance to Human Disease. New York: Marcel Dekker, Inc, 1997:15-35.

26. Golovkina T V, Chervonsky A, Dudley J P, Ross S R, Transgenic mouse mammary tumor virus superantigen expression prevents viral infection. Cell 1992;69:637-645.

27. Held W, Shakhov A N, Izui S, et al. Superantigen-reactive CD4+T cells are required to stimulate B cell after infection with mouse mammary tumor virus. J Exp Med 1993;177:359-366.

28. Held W, Waanders G A, Shakhov A N, Scarpellino L, Acha-Orbea H, MacDonald H R, Superantigen-induced immune stimulation amplifies mouse mammary tumor virus and allows virus transmission. Cell 1993;74:529-540.

29. Banki K, Maceda J, Hurley E, et al. Human T-cell lymphotropic virus (HTLV)—related endogenous sequence, HRES-1, encodes a 28-kDa protein: a possible autoantigen for HTLV-1 gag-reactive autoantibodies. Proc Natl Acad Sci 1992;89:1939-1943.

30. Conrad B, Weldmann E, Trucco G, et al. Evidence for superantigen involvement in insulin-dependent diabetes mellitus aetiology. Nature 1994;371:351-355.

31. Conrad B, Weissmahr R N, Boni J, Arcari R, Schupbach J, Mach B A, Human endogenous retroviral superantigen as candidate autoimmune gene in type I diabetes. Cell 1997;90:303-313.

32. Lower R, Tonjes R R, Boller K, et al. Development of insulin-dependent diabetes mellitus does not depend on specific expression of the endogenous retrovirus HERV-K. Cell 1998;95:11-14.

33. Dreyer E O, Muldiyarov P Y, Nassonova V A, Alekberova Z S. Endothelial inclusions and “nuclear bodies” in systemic lupus erythematosus. Ann Rheum Dis 1973;32:444-449.

34. Griffiths D J, Cooke S P, Herve C, et al. Detection of human retrovirus 5 in patients with arthritis and systemic lupus erythematosus. 1999;42:448-454.

35. Perl A, Colombo E, Dai H, et al. Antibody reactivity to the HRES-1 endogenous retroviral element indentifies a subset of patients with systemic lupus erythematosus and overlap syndromes. Arthritis Rheum 1995;38:1660-1671.

36. Furano A V. The biological properties and evolutionary dynamics of mammalian LINE-1 retrotransposons. Prog Nuc Acids Res and Mol Biol 2000;64:255-294.

37. Scott A F, Schmeckpeper B J, Abdelrazik M, et al. Origin of the human L1 elements: proposed pregenitor genes deduced from a consensus DNA sequence. Genomics 1987;1:1 13-125.

38. Hattori M, Kuhara S, Takenaka O, Sakaki Y. L1 family of repetitive DNA sequences in primates may be derived from a sequence encoding a reverse transcriptase-related protein. Nature 1986;321:625-628.

39. Moran J V, Holmes S E, Naas T P, DeBerardinis R J, Boeke J D, Kazazian H H. High frequency retrotransposition in cultured mammalian cells. Cell 1996;87:917-927.

40. Kazazian H H, Moran J V. The impact of L1 retrotransposons on the human genome. Nature Genet 1998;19:19-24.

41. Boeke J D, Pickeral O K. Retroshuffling the genomic deck. Nature 1999;398:108-111.

42. Boissinot S, ChevretP, Furano A V. L1 (LINE-1) retrotransposon evolution and amplification in recent human history. Mol Biol Evol 2000;17:915-928.

43. Sassaman D M, Dombroski B A, Moran J V, Kimberland M L, Naas T P, De Berardinis R J, Gabriel A, Swergold G D, Kazazian H H. Many human L1 elements are capable of retrotransposition. Nature Genet 1997; 16:37-43.

44. Kolosha V O, Martin S L. Polymorphic sequences encoding the first open reading frame protein from LINE-1 ribonucleoprotein particles. J Biol Chem 1995;270:2868-2873.

45. McMillan J B, Singer M F. Translation of the human LINE-1 element L1Hs. Proc Natl Acad Sci 1993;90:11533-11537.

46. Hohjoh H, Singer M F. Ribonuclease and high salt sensitivity of the ribonucleoprotein complex formed by the human LINE-1 retrotransposon. J Mol Biol 1997;271 :7-12.

47. Mathias S L, Scott A F, Kazazian H H, Boeke J D, Gabriel A. Reverse transcriptase encoded by a human transposable element. Science 1991;254:1808-1810.

48. Feng Q, Moran J V, Kazazian H H, Boeke J D. Human L1 retrotransposon encodes a conserved endonuclease required for retrotransposition. Cell 1996;87:905-916.

49. Boeke H D, Corces V G. Transcription and reverse transcription of retrotransposons. Annu Rev Microbiol 1989;43:403-434.

50. Dombroski B A, Scott A F, Kazazian H H. Two additional potential retrotransposons from a human L1 subfamily that contains an active retrotranposable element. Proc Natl Acad Sci 1993;90:6513-6517.

51. Esnault C, Maestre J, Hedimann T. Human LINE retrotransposons generate processed pseudogenes. Nature Genetics 2000;24:363-367.

52. Woodcock D M, Lawler C B, Linsenmeyer M E, Doherty J P, Warren W D. Asymmetric methylation in the hypermethylated CpG promoter region of the human L1 retrotransposon. J Biol Chem 1997;272:7810-7816.

53. Minakami R, Kurose K, Etoh K, Furuhata Y, Hattori M, Sakaki Y. Identification of an internal cis-element essential for the human L1 transcription and a nuclear factor(s) binding to the element. Nuc Acids Res 1992;20:3139-3145.

54. Leibold D M, Swergold G D, Singer M F, Thayer R E, Dombroski B A, Fanning T G. Translation of LINE-1 DNA elements in vitro and in human cells. Proc Natl Acad Sci 1990;87:6990-6994.

55. Kole L B, Haynes S R, Jelinek W R. Discrete and heterogeneous high molecular weight RNAs complementary to a long dispersed repeat family (a possible transposon) of human DNA. J Mol Biol 1983;165:257-286.

56. Branciforte D, Martins S L. Developmental and cell type specificity of LINE-1 expression in mouse testis: implications for transposition. Mol Cell Biol 1994;14:2584-2592.

57. Trelogan S A, Martin S L. Tightly regulated, developmentally specific expression of the first open reading frame from LINE-1 during mouse embryogenesis. Proc Natl Acad Sci 1995;92:1520-1524.

58. Neidhart M, Rethage J, Gay R E, Gay S. L1 retrotransposons in rheumatoid arthritis are related to genomic DNA hypomethylation and affect gene expression. Arthritis Rheum 1999;42:S248.

59. Kimberland M L, Divoky V, Prchal J, Schwahn U, Berger W, Kazazian H H. Full-length human L1 insertions retain the capacity for high frequency retrotransposition in cultured cells. Hum Mol Genet 1999;8:1557-1560.

60. Tend S-C, Kim B, Gabriel A. Retrotransposon reverse-transcriptase-mediated repair of chromosomal breaks. Nature 1996;383:641-644.

61. Kazazian H H, Wong C, Youssoufian H, Scott A F, Phillips D G, Antonarakis S E. Haemophilia A resulting from do novo insertion of L1 sequences represents a novel mechanism for mutation in man. Nature 1988;332:164-166.

62. Dombroski B A, Mathias S L, Nanthakumar E, Scott A F, Kazazian H H. Isolation of an active human transposable element. Science 1991;254:1805-1808.

63. Miki Y, Nishisho I, Horii A, Miyoshi Y, Utsunomiya J, Kinzler K W, Vogelstein B, Nakamura Y. Disruption of the APC gene by a retrotransposal insertion of L1 sequence in a colon cancer. Cancer Res 1992;52:643-645.

64. Holmes S E, Dombroski B A, Krebs C M, Boehm C D, Kazazian H H. A new retrotransposable human L1 element from the LRE2 locus on chromosome 1q produces a chimaeric insertion. Nature Genetics 1994;7:143-148.

65. Chu J L, Drappa J, Parnassa A, Elkon K B. The defect in Fas expression in MRL/lpr mice is associated with insertion of the retrotransposon, ETn. J Exp Med 1993; 178:723-730.

66. Quddus J, Johnson K J, Galvalchin J, Amento E P, Chrisp C E, Yung R L, Richardson B C. Treating activated CD4+ T cells with either of two distinct DNA methyltransferase inhibitors, 5-azacytidine or procainamide, is sufficient to cause a lupus-like disease in syngeneic mice. J Clin Invest 1993;92:38-53.

67. Dias Neto E, Correa R G, Verjovski-Almeida S, et al. Shotgun sequencing of the human transcriptome with ORF expressed sequence tags. Proc Natl Acad Sci 2000;97:3491-3496.

68. Woodcock D M, Williiamson M R, Doherty J P. A sensitive Rnase protection assay to detect transcripts from potentially functional human endogenous L1 retrotransposons. Biochem Biophys Res Comm 1996;222:460-465.

69. Crow M K: Mechanisms of T-helper cell activation and function in systemic lupus erythematosus. In: Lupus: Molecular and Cellular Pathogenesis. Edited by G. Kammer and G. Tsokos, Totowa, N J: Humana Press, Inc., pp. 231-256, 1999.

70. Tsokos, G C: Lymphocyte abnormalities in human lupus. Clin Immunol Immunopathol 1992;63:7-9.

71. Hohjoh H, Singer. Cytoplasmic ribonucleoprotein complexes containing human LINE-1 protein and RNA. EMBO J 1996;15:630-639.

72. Craft J, Mimori T, Olsen T L, Hardin J A. The U2 small nuclear ribonucleoprotein particle as an autoantigen. J Clin Invest 1988;81:1716-1724.

73. Herman J G, Graff J R, Myohanen S, Nelkin B D, Baylin S B. Methylation-specific PCR: a novel PCR assay for methylation status of CpG islands. Proc Natl Acad Sci USA 1996;93:9821-9826.

74. Malfoy B. The revival of DNA methylation. J Cell Sci 2000;113:3887-3888.

75. Kang S H, Choi H H, Kim S G, Jong H S, Kim N K, Kim S J, Bang Y J. Transcriptional inactivation of the tissue inhibitor of metalloproteinase-3 gene by DNA hypermethylation of the 5′ -CpG island in human gastric cancer cell lines. Int J Cancer 2000;86 :632-635.

76. Mahlknecht U, Hoelzer D. Histone acetylation modifiers in the pathogenesis of malignant disease. Mol Med 2000;6:623-644.

77. Hu E, Chen Z, Fredrickson T, Zhu Y, Kirkpatrick R, Zhang G F, Johanson K, Sung C M, Liu R, Winkler J. J Biol Chem 2000;275:15254-15264.

78. Richon V M, Sandhoff T W, Rifkind R A, Marks P A. Histone deacetylase inhibitor selectively induces p21WAF1 expression and gene-associated histone acetylation. Proc Natl Acad Sci USA 2000;97:10014-10019.

79. Smith L, Anderson K B, Hovgaard L, Jaroszewski J W. Rational selection of antisense oligonucleotide sequences. Eur J Pharm Sci 2000;11:191-198.

80. Mitchell P, Tollervey D. Curr Opin Genet Dev 2000;10:193-198, 2000.

81. Lai W S, Carballo E, Thorn J M, Kennington E A, Blackshear P J. J Biol Chem 2000;275:17827-17837.

82. Nielsen et al., Science 1991;254:1497.

83. Santer R, Rischewski J, Block G, Kinner M, Wendel U, Schaub J, Schneppenheim R. Molecular analysis in glycogen storage disease 1 non-A: DHPLC detection of the highly prevalent exon 8 mutations of the G6PT1 gene in German patients. Hum Mutat 2000;16:177.

84. Gurling H M, Kalsi G, Brynjolfson J, Sigmundsson T, Sherrington R, Mankoo B S, Read T, Murphy P, Blaveri E, McQuillin A, Petursson H, Curtis D. Genomewide genetic linkage analysis confirms the presence of susceptibility loci for schizophrenia, on chromosomes 1q32.2, 5q33.2, and 8p21-22 and provides support for linkage to schizophrenia, on chromosomes 11q23.2-24 and 20q12.1-11.23. Am J Hum Genet 2001;68:661-673.

85. Tchenio T, Casella J-F, Heidmann T. Members of the SRY family regulate the human LINE retrotransposons. Nucleic Acids Research 2000;28:411-415.

86. Tchenio T, Casella J-F, Heidmann T. Members of the SRY family Jawaheer D, Seldin M F, Amos C I, Chen W V, Shigeta R, Monteiro J, Kern M, Criswell L A, Albani S, Nelson J L, Clegg D O, Pope R, Schroeder H W Jr, Bridges S L Jr, Pisetsky D S, Ward R, Kastner D L, Wilder R L, Pincus T, Callahan L F, Flemming D, Wener M H, Gregersen P K. A genomewide screen in multiplex rheumatoid arthritis families suggests genetic overlap with other autoimmune diseases. Am J Hum Genet 2001;68:927-936.

87. St. George-Hyslop P H, Tanzi R E, Polinsky R J et al. The genetic defect causing familial Alzheimer's disease maps on chromosome 21. Science 235:885-890, 1987.

88. Lawrence S, Keats B J, Morton N E. The AD1 locus in familial Alzheimer disease. Ann Hum Genet 56:295-301, 1992.

89. Hardy J. The Alzheimer family of diseases: many etiologies, one pathogenesis? Proc Natl Acad Sci USA 94:2095-2097, 1997.

90. Pham C T, MacIvor D M, Hug B A, Heusel J W, Ley T J. Long-range disruption of gene expression by a selectable marker cassette. Proc Natl Acad Sci USA 93:13090-13095, 1996.

91. Gray-McGuire C, Moser K L, Gaffney P M, Kelly J, Yu H, Olson J M, Jedrey C M, Jacobs K B, Kimberly R P, Neas B R, Rich S S, Behrens T W, Harley J B. Genome scan of human systemic lupus erythematosus by regression modeling: evidence of linkage and epistasis at 4p16-15.2. Am J Hum Genet 67:1460-1469, 2000.

TABLE 1
Chromosomal location of proposed SLE disease loci and full length high fidelity
L1 elements. % sequence identity was determined in comparison to nt 1-884
of accession no. U09116.

| | | | Location of | | Distance of |
|---|---|---|---|---|---|
| | | | Nearest 98 | | L1 from |
| | | M Bases | or 99% | | Marker (in |
| Chromosome | Marker | From ptel | identity L1 | Gene containing L1 | M bases) |
| 1q | FCGR2A | 162.62 | 98 (167.4) | none | 4.78 |
| 1q | D1S1679 | 163.021 | 98 (167.4) | none | 4.34 |
| 1q | D1S213 | 182.58 | 98 (178.54) | LOC127055 | 4.04 |
| | | | 98 (182.4) | none | 0.18 |
| | | | 98 (182.6) | none | 0.02 |
| 1q | LAMC1 | 187.8 | 99 (185) | XPR1 | 2.8 |
| | | | 98 (186.7) | None | 1.1 |
| | | | 98 (189.8) | NIBAN | 0.02 |
| 1q | D1S2785 | 247 | 99 (244.3) | RYR2 | 2.7 |
| | | | 98 (244.3) | LOC128172 | 2.7 |
| | | | 98 (248.7) | none | 1.7 |
| 4p | D4S403 | 28 | 98 (28) | none | 0 |
| | | | 98 (31) | none | 3 |
| 4q | D4S2368 | 168.5 | 98 (166.2) | Similar to testican | 2.3 |
| | | | 99 (169.2) | NEK1 | 0.7 |
| 6p | D6S2410 | 53.57 | 98 (47.72) | SUPT3H | 5.58 |
| | | | 99 (48.8) | none | 4.77 |
| 16q | D16S415 | 54.31 | 98 (54.6) | MGC5149 | 0.29 |
| | D16S503 | 65.28 | 98 (67.7) | none | 2.42 |
| | | | 99 (70.5) | none | 5.22 |

TABLE 2
Analysis of L1 element expression in the genomic regions neighboring
schizophrenia disease loci. Seven loc
on 5 chromosomes were studied and are darkly highlighted.

| | | L1 | | | |
|---|---|---|---|---|---|
| | | % density | | | |
| Location | | to 1-884 of | | | |
| Marker | | U09116 | | | |
| (M bases | | location on | | Description or | Additional |
| from ptel) | Contig | contig | Gene | mRNA | Description |
| 162.7 | NT_004406 | 91cds | LOC127384 | XM_060457 | |
| | | 862,690 | | Sim to serum | |
| | | 88,85 | | amyloid P/crp | |
| | | | | family | |
| 163.7 | NT_026945 | 88cds | L0C126821 | XM_060193 | |
| | | 16.919 | | Dead/deah box | |
| | | | | helicase | |
| 165.4 | NT_004745 | 97 | | | |
| D1s1679 | | 89cds | ATF6 | | |
| | | 2,404,741 | | | |
| | | 88,87,87 | | | |
| | | 87cds | DDR2 | | |
| | | 1,349,888 | | | |
| 167.1 | NT_004648 | 95,90,90,87 | | | |
| | | 88cds | | | |
| | | 1,489,481 | ALDH9A1 | | |
| D1S196 | NT_004668 | 98 | Near | Sim to Ewings | |
| Z16503 | AL031733 | 4,217,155 | FLJ00024? | sarcoma oncogene | |
| 170.1 | (2,860,000) | 97,95 | LOC92233 | | |
| (2,830,998) | | | LOC127890 | XM_059191 | |
| | | 95cds | LOC127910 | XM_060726 | |
| | | 6,071,165 | | | |
| | | 94 | | Nucleoside | |
| | | 94cds | NME7 | diphosphate | |
| | | 1,194759 | | kinase | |
| | | 94cds | LOC127914 | XM_072920 | |
| | | 6,774,236 | | | |
| | | 92 | | | |
| | | 91cds | NME7 | | |
| | | 1,172,510 | | | |
| | | 90 | | | |
| | | 90cds | LOC127911 | XM_072918 | |
| | | 6,314,453 | | | |
| | | 89cds | ATF6 | CAMP-dep tran | |
| | | 9,220,139 | | factor | |
| | | 88,87,87 | | | |
| | | 86cds | LOC127891 | XM_059193 | |
| | | 4,403,629 | | | |
| | | 84cds | LOC127889 | XM_072913 | |
| | | 4,149,207 | | | |
| | | 85 | | | |
| | | 83cds | LOC127906 | XM_127906 | |
| | | 5,501,845 | | | |
| | | 90cds | LOC57821 | NM_021179 | |
| | | 1,047,463 | | | |
| | | 87cds | LOC127934 | XM_060744 | |
| | | 8,510,019 | jk | | |
| | | 87cds | | Discoidin domain | |
| | | 7,810,544 | DDR2 | rec fam 2 | |
| | | | | Neurotrophic tyr | |
| | | | | kinase rec rel3 | |
| | | 88cds | | | |
| | | 4,815,922 | ALDH9A1 | | |
| | | 89cds | | | |
| | | 3,680,941 | LOC127885 | XM_060709 | |
| | | 85cds | | | |
| | | 9,359,548 | LOC127944 | XM_060702 | |
| | | | Near fcfr2b | Rib prot L31 | |
| | | 85cds | | | |
| | | 1,467,216 | LOC127874 | XM_072909 | |
| | | 86cds | | | |
| | | 6,335,176 | LOC127911 | XM_072918 | |
| | | 79cds | | | |
| | | 6,030,189 | LOC127910 | XM_060726 | |
| | | 83cds | | | |
| | | 6,578,251 | LOC127912 | XM_060727 | |
| | | 86cds | | Sim to adenyl cyc | |
| | | 6,540,496 | LOC127912 | | |
| | | 81 cds | | | |
| | | 1,040,525 | LOC57821 | XM_001574 | |
| | | 81cds | | | |
| | | 399,716 | KIFAP3 | Kinensin assoc | |
| | | | | protein 3 | |
| | | | | Sing GDS-assoc | |
| | | | | prot SMAP - | |
| | | | | cerebellum | |
| 174.7 | NT_029874 | 97cds | LOC127090 | XM_060336 | Ganglioside gm2 |
| | | 286,649 | | Shingolipid | activator precursor |
| | | | | activator prot 3 | protein 3 |
| | | 96cds | LOC127105 | XM_072723 | |
| | | 1,900,362 | | | |
| | | 95cds | LOC127100 | XM_044463 | |
| | | 968,927 | | | |
| | | 94 | | | |
| | | 93cds | LOC127100 | | |
| | | 1,227,344 | | | |
| | | 89 | | | |
| | | 87cds | LOC127109 | XM_060332 | |
| | | 2,235,511 | | Sum to rib prot | |
| | | | | L26 | |
| | | 87cds | LOC127109 | | |
| | | 2,140,534 | | | |
| | | 86cds | C1ORF9 | XM_002138 | |
| | | 1,510,476 | | | |
| | | 90cds | LOC92346 | XM_044465 | |
| | | 1,344,710 | | | |
| | | 88cds | LOC127091 | XM_043678 | |
| | | 469,620 | KIAA1096 | | |
| | | 87cds | LOC127100 | | |
| | | 1,215,001 | | | |
| | | 81cds | LOC127100 | | |
| | | 994,144 | | | |
| | | 79cds | LOC127100 | | |
| | | 937,597 | | | |
| | | 84cds | LOC127109 | | |
| | | 2,231,864 | | | |
| 176 | NT_021942 | 81 | | | |
| 176.6 | NT_029873 | 98 | Near | XM_031117 | Phosphotyr |
| | | 4281 | LOC90354 | Sim to Rab6 | Binding dom |
| | | 97cds | LOC90354 | GTPase act | |
| | | 216,844 | | protein | |
| | | 96,86 | | | |
| 177.6 | NT_004470 | 88 | | | |
| 178 | NT_029868 | 98cds | LOC127055 | XM_060298 | Ribosomal L13e |
| | | 302,955 | | | like |
| | | 95cds | FLJ10416 | Sim to | |
| | | 46,079 | | photomorphogenic | |
| | | 93 | | prot | |
| | | 92cds | ″ | | |
| | | 30,009 | | | |
| | | 78cds | LOC127056 | XM_072714 | |
| | | 479,243 | | | |
| 179 | NT_004398 | bit | | | |
| 112.1 | NT_028147 | 98cds | LOC132718 | XM_076833 | |
| | | 2,142,361 | | | |
| | | 96cds | LOC132709 | XM_067997 | Kruppel-assoc box |
| | | 563,171 | FLJ10891 | | |
| | | 95 | | | |
| | | 94cds | LOC132712 | XM_076837 | |
| | | 1,036.908 | | | |
| | | 94 | | | |
| | | 93cds | LOC132714 | XM_067992 | |
| | | 1,520,639 | | | |
| | | 93,92 | | | |
| | | 90cds | LOC132715 | XM_076830 | |
| | | 1,621.371 | | | |
| | | 90cds | LOC132715 | | |
| | | 1,596.843 | | | |
| | | 89 | | | GNS1_SUR4 |
| | | 87cds | LCE | XM_045523 | Long chain fatty |
| | | 228,178 | | | Acyl elongase |
| | | 86cds | LOC132707 | XM_067995 | |
| | | 479,677 | Sim to Ac- | | |
| | | | Liketrans e1 | | |
| | | 84cds | LOC132717 | XM_076832 | |
| | | 1.928,148 | | | |
| | | 93cds | LOC132714 | XM_067992 | |
| | | 1.431,709 | | | |
| | | 87cds | LOC132713 | XM_067998 | |
| | | 1.260,497 | | | |
| | | 87cds | EGF | XM_003608 | Mitogenic; stim |
| | | 91.217 | | | phos of H3; LDL |
| | | | | | rec domain |
| | | | | | Protect from LDL |
| | | | | | oxidation |
| | | 78cds | LOC132717 | XM_076832 | |
| | | 1.864,383 | | | |
| 113.9 | NT_006371 | 94cds | CAMK2D | | Calcium/calmoduli |
| | | 574.774 | | | n |
| | | | | | Dep kinase delta2 |
| | | | | | May be involved in |
| | | 92cds | LOC133294 | XM_077029-x | neuron signaling |
| | | 517,127 | | | hippo/pyramidal |
| | | | | | cells |
| 114.8 | NT_022790 | 94 | | | |
| | | 87cds | LOC132392 | XM_067820 | |
| | | 649.533 | | | |
| | | 86 | | | |
| D4S430 | NT_029273 | 92,90,86 | | | |
| Z17169 | AC053545 | | | | |
| 115.4 | | | | | |
| (111,507) | | | | | |
| 116 | NT_028173 | 94cds | LOC132747 | XM_076846 | |
| | | 45,911 | | | |
| 116.2 | NT_022931 | Nss | | | |
| 116.7 | NT_022989 | 87.88 | | | |
| | | 83cds (17) | | | |
| 117.4 | NT_016599 | 97 | | | |
| 160.7 | NT_023152 | 98 | Near | XM_077106 | |
| | | 1.764,039 | LOC133516 | | |
| | | 98 | | | |
| | | 2.130,593 | | | |
| | | 98cds(11 | LOC133511 | XM_068388 | |
| | | 446,551 | | Sim pdgf rec like | |
| | | 97,97 | | Prot | |
| | | 94cds | LOC133515 | XM_077105 | |
| | | 1.473,285 | | | |
| | | 94 | | | |
| | | 92cds | LOC133514 | XM_077104 | |
| | | 1,275,463 | | | |
| | | 92cds | ATP10B | ATPase | |
| | | 258,166 | | | |
| | | 8887 | | | |
| | | 87cds | LOC133515 | XM_077105 | |
| | | 1,399,629 | | | |
| | | 87 | | | |
| | | 82cds | GLRA1 | Glycine rec alpha1 | Mutated in hyper- |
| | | 1,884,591 | | Startle dis/stiff | Ekplexia (startle |
| | | | | man syndrome | dis) - exon 6 |
| | | 81cds | LOC133512 | XM_077102 | |
| | | 530,876 | | | |
| | | 80cds | ATP10B | | |
| | | 250,195 | | | |
| | | 81cds | LOC133515 | | |
| | | 1,454,062 | | | |
| 163 | NT_006788 | 98 | Near | XM_068801 | |
| | | 1,682,752 | LOC134347 | | |
| | | 86cds | SGCD | Sarcoglycan | Knock out has |
| | | 1,106,028 | | Dystrophin assoc | cardiomyopathy |
| | | | | gp | |
| | | 85cds | LOC134344 | XM_056963 | |
| | | 377,215 | | | |
| | | 88cds | SGCD | | |
| | | 1,135,192 | | | |
| | | 86cds | LOC134344 | | |
| | | 370,363 | | | |
| 164.5 | NT_029980 | Nss | | | |
| 165.4 | NT_007006 | 85,88,92,81 | | | |
| 166.5 | NT_029289 | Nss | | | |
| 167 | NT_023255 | Nss | | | |
| 167.3 | NT_025716 | 94 | | | |
| D5s422 | NT_006983 | 94cds | LOC134485 | | |
| Z16965 | AC010602 | 184,438 | | | |
| (167.97) | (438,043) | 91 | | | |
| (380,023) | | 87cds | LOC134487 | | |
| DSS400 | | 349,741 | | | |
| D5S410 | | 86cds | LOC134491 | | |
| | | 807,389 | | | |
| | | 82cds | LOC134491 | | |
| | | 849,502 | | | |
| | | 86cds | LOC 134488 | Sim to fibulin | Ig domain |
| | | 619,246 | | | |
| | | 84cds | LOC134486 | | |
| | | 278,288 | | | |
| 172 | NT_006907 | 88,87,87,85 | | | |
| 175.5 | NT_023154 | 96 | | | |
| | | 87cds | LOC133532 | XM_077110 | |
| | | 1,084,950 | | | |
| | | 89cds | LOC133527 | XM_068392 | |
| | | 596,953 | | | |
| | | 84cds | LOC133530 | XM_077109 | |
| | | 955,499 | | | |
| | | 75cds | LOC133527 | | |
| | | 675,271 | | | |
| 0 | NT_008060 | 86,90 | | | |
| 0.7 | NT_023779 | 91 | | | |
| 1.5 | NT_023744 | 97cds(31- | LOC137257 | XM_051541 | Interacts with Hom |
| | | 463,266 | | DLGAP2 | of Drosophila discs |
| | | | | Concentrated in | tumor supp prot are |
| | | | | synaptic junctions; | assoc with NMDA |
| | | | | Coprecip with | rec and K channel |
| | | | | GRIN2A | prot; brain and |
| | | | | (NMDAR2), | kidney; cand for |
| | | | | APC, and beta- | progressive |
| | | | | catenin; colocal | epilepsy with |
| | | | | with DLG4 and | mental retardation |
| | | | | GRIN2A in hippo | |
| 2.2 | NT_008087 | 94,93 | | | |
| | | 85cds | LOC137745 | XM_070579 | |
| | | 461,662 | | Sim to Pto kinase | |
| | | 81cds (9- | ″ | interactor | |
| | | 448,734 | | | |
| 3.8 | NT_008227 | 94cds | LOC137940 | XM_078497 | |
| | | 56,458 | | | |
| | | 86cds | CSMD1 | XM_054838 | CUB (found in C1r, |
| | | 1,825,757 | | | C1s, uEGF, and |
| | | 85cds | ″ | | BMP) and sushi |
| | | 1,786,802 | | | multiple domains 1; |
| | | 87cds | ″ | | CCP domain |
| | | 1,625,088 | | | (abundant in |
| | | 94 | | | complement control |
| | | 94cds | ″ | | proteins) |
| | | 1,805,349 | | | |
| | | 84cds | LOC137946 | XM_078501 | |
| | | 342,566 | | | |
| 5.6 | NT_023736 | 88,87 | | | |
| 6.3 | NT_023904 | Nss | | | |
| D8S503 | NT_019483 | 98cds | LOC137062 | XM_078189 | |
| Z23470 | | 2,224,912 | | | |
| 7.28 | | 86,86 | | | |
| (2,564,262) | | 85cds | LOC137065 | XM_070213 | |
| | | 2,492,759 | | | |
| | | 92 | | | |
| 10 | NT_023868 | Nss | | | |
| 10.2 | NT_030035 | 86 | | | |
| 10.4 | NT_026362 | 85 | | | |
| 10.8 | NT_008010 | 85 | | | |
| 11.2 | NT_008175 | Nss | | | |
| 11.6 | NT_008128 | 92 | | | |
| 12.1 | NT_008004 | 90cds | FDFT1 | XM_035680 | Farnesyl- |
| | | 93,373 | | Cholesterol | diphosphate |
| | | | | biosynthesis | farnesyltransferase |
| | | | | Rec syndrome | 1 |
| 12.4 | NT_029349 | Nss | | | |
| 13 | NT_008161 | 90cds | LOC137868 | XM_059926 | Sim to zeta |
| | | 1,633,926 | | | sarcoglycan |
| 13.6 | NT_030030 | Nss | | | |
| 21.9 | NT_023704 | 84cds | | | |
| | | 141,075 | | | |
| | | 86 | | | |
| 22.4 | NT_008205 | 77 | | | |
| 22.9 | NT_008300 | 93cds | | | |
| | | 319,091 | | | |
| 23.3 | NT_008047 | Nss | | | |
| 23.8 | NT_023664 | Nss | | | |
| 24.6 | NT_008130 | 95cds | LOC137815 | x | |
| | | 680,765 | | | |
| D8S1771 | NT_023666 | 91,93 | | | |
| Z53561 | | 91cds | LOC137104 | | |
| 26.48 | | 1,277,844 | | | |
| (517,449) | | 97cds | LOC137097 | | |
| | | 361,474 | | | |
| | | 86 | | | |
| 27.3 | NT_030023 | 98cds | BNIP3L | BCL2/adenovirus | Mitochondria; |
| | | 332,957 | | E1B 19kD- | colacalizes with 60 |
| | | | | interacting protein | kD hsp-1; |
| | | | | 3-like family | proapoptotic fin; |
| | | | | propoptotic | binds BCL2 family |
| | | | | | members |
| 27.9 | NT_008139 | 99cds | LOC137822 | XM_070621 | ? |
| | | 611,028 | | | |
| | | 87,87,86 | | | |
| 29.5 | NT_007988 | 93 | | | |
| 31 | NT_007993 | 85cds | WRN | Werner syndrome | Scleroderma-like |
| | | 185,359 | RECQL2 | Homolog of RecQ | skin changes; |
| | | | | helicase; 3′ to 5′ | subcutaneous |
| | | | | exonuclease; | calcification; |
| | | | | Nucleolar; homol | premature |
| | | | | gene in C. elegans | arterioscl, DM; |
| | | | | implicated in | acceleration of |
| | | | | silencing of | telomere-driven |
| | | | | transposons and | replicative |
| | | | | RNA interference | senescence |
| 101-125 | NT_009151 | | | | |
| 125 | NT_030107 | Nss | | | |
| 129 | NT_009215 | 94cds | TECTA | Tectorin alpha | deafness |
| | | 1,920,753 | | | |
| | | 88 | | | |
| | | 81cds | GRIK4 | Glutamate rec | |
| | | 2,324,593 | | | |
| | | 99cds(118- | LOC 120493 | Sim to LINE RT | XP_062069 |
| | | 1,215.303 | | homolog | |
| D11S934 | NT_009115 | 98 | Near PIG8 | P53-induced gene | |
| Z17119 | | 827,840 | 784,102 | 8; activates an | |
| 132.8 | | | | apoptotic pathway | |
| (1,384,127) | | | LOC120253 | | Sim to surf gp, Ig |
| D11s925 | | | | | fam |
| | | 98 | | | |
| | | 494,488 | FEZ1 | Fasc and elong | Axonal outgrowth |
| | | | 446,059 | Protein zeta 1 | |
| | | | Near | | |
| | | | LOC120251 | 527,380 | Sim to etoposide- |
| | | | | | induced mRNA |
| | | | ITM1 | 550,705 | Integral mem prot |
| | | | CHEK1 | 584,640 | checkpoint |
| | | | | | monitors meiotic |
| | | | | | recombination |
| | | 97cds | LOC120271 | XM_073548 | |
| | | 3.036,022 | | | |
| | | 94cds | LOC120267 | XM_073545 | |
| | | 2,758,023 | | | |
| | | 93,93,92,88 | | | |
| | | 83,83,83 | | | |
| | | 84cds | LOC120293 | | |
| | | 4,978,908 | | | |
| | | 80cds | LOC120289 | | |
| | | 4,773.040 | | | |
| | | 82cds | LOC120253 | | |
| | | 880,521 | | | |
| | | 96cds | LOC120279 | | |
| | | 3,694,083 | | | |
| | | 96cds | LOC120275 | | |
| | | 3,364,553 | | | |
| 138.3 | NT_009056 | 93cds | LOC120214 | XM_07527 | |
| | | 1,156,972 | | | |
| 140.3 | NT_024213 | nss | | | |
| 141 | NT_009276 | 95,85 | | | |
| 142 | NT_024180 | bits | | | |
| D20S112 | NT_011387 | 99 | Near | 23,329,380 | |
| 17.25M | AL031675 | 23,311,773 | LOC128817 | | |
| | | 99 | Near | 12,701,572 | |
| | | 12,692,681 | LOC128729 | | |
| | | 99cds | | sim to | |
| | | 11,518,452 | PGAM-B | phosphoglycerate | |
| | | | | mutase, brain | |
| | | 97,96,96,96 | | form | |
| | | 95cds | LOC128685 | | |
| | | 6,207,479 | | | |
| | | 95 | | | |
| | | 94cds | LOC128795 | | |
| | | 21,802,506 | | | |
| | | 94,94 | | | |
| | | 99 | near | 6,805,558 | |
| | | 7,007,833 | LOC128691 | | |
| | | 93cds | LOC128726 | | |
| | | 12,015341 | | | |
| | | 94,92,91 | | | |
| | | 89cds | LOC128759 | | |
| | | 18,218,585 | | | |
| | | 88cds | LOC128812 | | |
| | | 22,879,343 | | | |
| | | 87,87 | | | |
| | | 84cds | LOC128749 | | |
| | | 16,679,616 | | | |
| | | 90cds | LOC128753 | | |
| | | 17,006,560 | | | |
| | | 88cds | LOC128727 | | |
| | | 12,377,291 | | | |
| | | 88cds | LOC128649 | | |
| | | 2,150,115 | | | |
| | | 88cds | FLJ20212? | | |
| | | 13,478,906 | | | |
| | | 88cds | | | Hi in islets of Lang |
| | | 17,296,781 | LOC96688 | Sim to | |
| | | | (17.15- | neuroendocrine | |
| | | | 17.4)XM_{—} | convertase 2 | |
| | | | 046457 | precursor (NEC 2) | |
| | | 87cds | LOC128794 | | |
| | | 21,739,101 | | | |
| | | 86cds | LOC128666 | | |
| | | 4,287,601 | | Sim to SEL1L | Suppressor of lin-12 |
| | | 87cds | DJ842G6.2 | XM_046437 | Pancreas; negative |
| | | 13,357,375 | | | Regulator of notch |
| | | 84cds | LOC128714 | | pathway |
| | | 11,038,855 | | | |

TABLE 3
Contigs on chromosome 1q containing full-length L1 element sequences.
Nss signifies no sequence homology to nt 1-884 of accession no. U09116.

| | | | % identity to | | | |
|---|---|---|---|---|---|---|
| | | | U09116 1-883 | | | |
| Chrom. | M Bases | | Location of L1 | Gene or | Description of Gene | Additional |
| Location | From ptel | Contig | in contig | LocusLink | Or similar Gene | Description |
| 1q | 148.2 | NT_022052? | 89 | | | |
| | | NT_004434+ | 95cds | LOC127469 | Sim to | |
| | | | 435,546 | (148.75) | bM332P19.3 - | |
| | | | 94,89 | | novel 7 | |
| | | | 88cds | as above | transmembrane | |
| | | | 414,715 | | receptor rhodopsin | |
| | | | 82cds | LOC127475 | family olfactory rec | |
| | | | 874,365 | (149.2) | like protein | |
| | | | 82cds | as above | | |
| | | | 867,310 | | | |
| | | NT_030568 | 88 | | | |
| | 150 | NT_029226− | 89cds | Cezanne | Cellular zinc finger | |
| | | | 737,990 | (149.8) | anti-NF kappa B- | |
| | | | | | A20-like zinc | |
| | | | | | finger domain | |
| | | | | | (inhib of cell death) | |
| | | NT_004811 | Bitcds | | | |
| | | NT_021907+ | 89cds | LOC126632 | | |
| | | | 787,136 | | | |
| | | NT_004441 | 82,87 | | | |
| | | NT_030577? | Nss | | | |
| | 154.5 | NT_021933 | Nss | | | |
| | | NT_004524 | Nss | | | |
| 1q22 | | NT_004858+ | 85cds | LOC128249 | Sim to cell surface | XM_060902 |
| | | | 2,260,795 | (157.95) | molecule Ly-9 | (Ly-9 = |
| | | | 94cds | ″ | (CD229) | AF244129; |
| | | | 2,352,293 | | -Ig superfamily | 29% identical) |
| | | | 87cds | ″ | (others are CD2, | |
| | | | 2,322,154 | | CD48, CD58) | |
| | | | 84cds | ″ | | |
| | | | 2,355,589 | | | |
| | | | 84cds | | | |
| | | | 2,300,855 | | | |
| | | NT_019291− | Bits | | | |
| | 159 | NT_004982+ | 94cds | LOC128375 | Sim to gamma | |
| | | | 1,245,500 | (159.83) | interferon inducible | |
| | | | 93,88,85,85.84 | | protein 16 | |
| | | NT_030566− | 94 | | | |
| | | NT_026222? | Nss | | | |
| | 161 | NT_004406− | 91cds | LOC127384 | Sim to porcine | Binds to lipid of |
| | | | 862,690 | (160.62) | Amyloid A protein | apoptotic cells |
| | | | 88,85 | | | |
| | | NT_026945+ | 88cds | LOC126821 | | |
| | | | 16,919 | | | |
| D1S1679 | 163.021 | NT_004668 | | | | |
| | 162-171 | NT_004668− | 98(167.4) | Near | Sim to Ewings | Breakpoint |
| | | | 4,217,155 | FLJ00024? | sarcoma oncogene | region |
| | | | 97,95 | LOC92233 | | |
| | | | | LOC127890 | XM_059191 | |
| | | | −95cds | LOC127910 | XM_060726 | No con dom; |
| | | | 6,071,165 | | | drohom |
| | | | 94 | | Nucleoside | |
| | | | 94cds | NME7 | diphosphate kinase | |
| | | | 1,194759 | (170.2-.5) | | |
| | | | −94cds | LOC127914 | XM_072920 | |
| | | | 6,774,236 | (964.85) | | |
| | | | 92 | | | |
| | | | 91cds | NME7 | | |
| | | | 1,172,510 | (170.5 | | |
| | | | 90 | | | |
| | | | −90cds | LOC127911 | XM)072918 | |
| | | | 6,314,453 | | | |
| | | | −89cds | ATF6 | CAMP-dep tran | Unfolded prot |
| | | | 9,220,139 | (162.3-.4) | factor; binds SRF | response |
| | | | 88,87,87 | | | FLJ21522 |
| | | | −86cds | LOC127891 | XM_059193 | Sim FLJ00024 |
| | | | 4,403,629 | (167.2) | | SH3 dom |
| | | | | | | Phosphotyr |
| | | | | | | Interaction |
| | | | | | | Sim transporter |
| | | | 84cds | LOC127889 | XM_072913 | |
| | | | 4,149,207 | | | |
| | | | 85 | | | |
| | | | −83cds | LOC 127906 | XM_127906 | |
| | | | 5,501,845 | | | |
| | | | −0cds | LOC57821 | NM_021179 | |
| | | | 1,047,463 | | | |
| | | | −87cds | LOC 127934 | XM_060744 | |
| | | | 8,510,019 | | | |
| | | | 87cds | | Discoidin domain | Extracell factor |
| | | | 7,810,544 | DDR2 | rec fam 2 | VIII-like |
| | | | | (163.8) | Neurotrophic tyr | domain; act by |
| | | | | | kinase tee rel3 | collagen |
| | | | 88cds | | | |
| | | | 4,815,922 | ALDH9A1 | Catal dehydrogen- | |
| | | | −89cds | (166.75) | Ation of GABA | |
| | | | 3,680,941 | LOC127885 | XM_060709 | Sim toFlavin |
| | | | | | | cont |
| | | | −85cds | LOC127944 | XM_060702 | Monooxygen5 |
| | | | 9,359,548 | Near fcfr2b | Rib prot L31 | |
| | | | | (163.22) | | |
| | | | −85cds | LOC127874 | XM_072909 | |
| | | | 1,467,216 | | | |
| | | | −86cds | LOC127911 | XM_072918 | |
| | | | 6,335,176 | (165.4) | | |
| | | | −79cds | LOC127910 | XM_060726 | |
| | | | 6,030,189 | (165.5) | | |
| | | | −3cds? | LOC127912 | XM_060727 | |
| | | | 6,578,251 | (165.05) | Sim to adenyl cyc | |
| | | | −86cds? | LOC127912 | | |
| | | | 6,540,496 | | | |
| | | | −81cds | LOC57821 | XM_001574 | |
| | | | 1,040,525 | | | |
| | | | 81cds | KIFAP3 | Kinensin assoc | |
| | | | 399,716 | (171.08- | protein 3 | |
| | | | | 171.2) | Sing GDS-assoc | Armadillo |
| | | | | | prot SMAP - | repeats; phos by |
| | | | | | cerebellum | v-src |
| D1S196 | 168.76 | | | | | |
| | | NT_004732− | 87,87,89,87 | | | |
| | | NT_030564− | 87cds | LOC127144 | | |
| | | | 201,283 | (174.1) | | |
| | 175.5 | NT_029874+ | 97cds | LOC127090 | Sim to ganglioside | Ganglioside |
| | | | 286,649 | (164.57) | GM2 activator | gm2 activator |
| | | | | | precursor (SAP-2) | precursor |
| | | | | | | protein 3 |
| | | | 96cds | LOC127105 | XM_072723 | |
| | | | 1,900,362 | | | |
| | | | 95cds | LOC127100 | XM_044463 | XP_044463 |
| | | | 969,927 | | | KIAA0820 |
| | | | 94 | (175.0) | | Dynamin |
| | | | 93cds | LOC127100 | | domain GTPase |
| | | | 1,227,344 | | | effector domain |
| | | | 89 | | | (mediate vesicle |
| | | | 87cds | LOC127109 | XM_060332 | trafficing); |
| | | | 2,235,511 | | Sim to rib prot L26 | pleckstrin homo |
| | | | 87cds | LOC127109 | | |
| | | | 2,140,534 | | | |
| | | | 86cds | C1ORF9 | XM_002138 | |
| | | | 1.510,476 | | | |
| | | | 90cds | LOC92346 | XM_044465 | |
| | | | 1,344,710 | | | |
| | | | 88cds | LOC127091 | XM_043678 | |
| | | | 469,620 | KIAA1096 | | |
| | | | 87cds | LOC127100 | | |
| | | | 1,215,001 | | | |
| | | | 81cds | LOC127100 | | |
| | | | 994,144 | | | |
| | | | 79cds | LOC127100 | | |
| | | | 937,597 | (175.6) | | |
| | | | 84cds | LOC127109 | | |
| | | | 2,231,864 | | | |
| | | NT_021942? | Bits | | | |
| | | NT_030570? | Nss | | | |
| | 177.5 | NT_004470− | Bits | | | |
| | | NT_029868+ | 98cds | LOC127055 | | |
| | | | 302,955 | (178.54) | | |
| | | | 95cds | FLJ10416 | Sim to | |
| | | | 46,079 | (178.3) | photomorph- | |
| | | | 93 | | Ogenic prot | |
| | | | 92cds | FLJ10416 | Sim to | |
| | | | 30,009 | | photomorph- | |
| | | | 78cds | LOC127056 | Ogenic prot | |
| | | | 479,243 | | | |
| D1S1589 | | NT_026949+ | 98(182.4) | | | |
| (182.58) | | | 3,396,062 | | | |
| | | | 98cds (182.6) | ? | | |
| | | | 3,597,400 | | | |
| | | | 97 | | | |
| | | | 97cds | LOC90354 | | |
| | | | 3,809,963 | | | |
| | | | 96cds | As above | | |
| | | | 3,954,485 | | | |
| | | | 96 | | | |
| | | | 95cds | LOC126850 | | |
| | | | 1,184,773 | | | |
| | | | 94 | | | |
| | | | 93cds(31 | LOC126858 | | |
| | | | 2,588,833 | | | |
| | | | 91,90 | | | |
| | | | 88cds | LOC126850 | | |
| | | | 1,228,754 | | | |
| | | | 86,86 | | | |
| | | | 83cds | FLJ10244 | Guanine nuc | Like SOS |
| | | | 2,432,829 | | exchange factor | |
| | | | 83cds | LOC126850 | | |
| | | | 1,243,379 | | | |
| | | | 89cds | NPHS2 | podocin | Neplirotic |
| | | | 3,170,271 | (181.85) | | syndrome |
| | 185 | NT_004552+ | 99cds | XPR1 | Xenotropic and | |
| | | | 1,317,693 | (184.28) | polytropic | |
| | | | 87 | | retrovirus receptor | |
| | | | 86cds | LOC127663 | ??? | |
| | | | 2,407,307 | (185.7) | | |
| | | | 86cds | | | |
| | | | 2,445,258 | as above | | |
| | | | 79cds | | | |
| | | | 2,487,143 | as above | | |
| | | | 88cds | MGC2404 | | |
| | | | 778,461 | (183.93) | | |
| | | NT_029219+ | 98(186.7) | Near | Sim to embigin | |
| | | | 85,973 | LOC126918 | | |
| | | | 96,92 | (186.7) | | |
| | | | 96cds | LOC126920 | Sim to KIAA0456 | |
| | | | 379,853 | (187.15) | | |
| | | NT_029880? | Nss | | | |
| LAMC1 | 188.6 | NT_029864− | 93cds | LOC127028 | | |
| (187.8) | | | 983,033 | (188.4) | | |
| | | | 89,89 | | | |
| | | | 78cds | as above | | |
| | | | 956,095 | | | |
| | | NT_004487+ | 98cds (189.8) | C1orf24 | Niban | |
| | | | 340,228 | | | |
| | | | 98 | Near | | |
| | | | 3,128,788 | LOC127523 | | |
| | | | 98 | Near | | |
| | | | 2,880,876 | LOC127522 | | |
| | | | | And LOC | | |
| | | | | 127521 | | |
| | | | 95cds | C1orf26 | | |
| | | | 752,376 | | | |
| | | | 95,95,93,92 | | | |
| | | | 91cds | FIBL-6 | Sim to basement | |
| | | | 1,658,122 | (190.65) | membrane specific | |
| | | | | | heparan sulfate PG | |
| | | | | | core protein | |
| | | | | | precursor | |
| | | | 92cds | LOC127514 | | |
| | | | 879,310 | (190.3) | | |
| | | | 85cds | LOC127519 | Near PRG4 | |
| | | | 1,757,301 | | | |
| | | NT_021972− | 86cdsbit | | | |
| | | | 272,0623 | | | |
| | 195 | NT_021905− | 93,91,90 | | | |
| | | NT_004671+ | 98cds | LOC127964 | Sim to ribosomal | |
| | | | 2,492,213 | | protL23A | |
| | | | 97 | | | |
| | | | 96cds | LOC127946 | -sim to myomegalin | |
| | | | 6924 | | | |
| | | | 96 | | | |
| | 200 | NT_004599+ | 84,82 | | | |
| | | NT_021909+ | Bits | | | |
| | | NT_004416− | 99 | Near | Sim to H factor 1 - | Also near factor |
| | | | 392,676 | LOC127387 | complement | H |
| | | | | (202.65) | Isoform 1 | |
| | | | | | -sim to complement | |
| | | | 93cds | LOC127388 | factor H-rel proteiin | |
| | | | 55 1,372 | (202.5) | 3 precursor-FUR- | sushi |
| | | | | | 3 | |
| | | NT_029862+ | 98 | Near | | |
| | | | 797,582 | LOC127012 | | |
| | | | 93 | (204.1) | | |
| | | | 93cds | LOC127012 | | |
| | | | 931,447 | | | |
| | | | 88cds | FHRS | Factor H-rel | |
| | | | 83,139 | (203.22) | Prot 5 | |
| | | | 83cds | | | |
| | | | 255,648 | | | |
| | | | 92cds | LOC127012 | | |
| | | | 958,777 | | | |
| | | | 96cds | F13B | Coag factor13B | |
| | | | 134,515 | (203.28) | | |
| | | NT_030560 | 88 | | | |
| | | | 82cds | LOC127117 | | |
| | | | 258,938 | | | |
| | 207.5 | NT_004680+ | 92,92,89,86 | | | |
| D1S1678 | | NT_004662− | 93cds | LOC127827 | Sim to gp330 | |
| | | | 676,800 | (212.45) | | |
| | | | 92cds | LOC93273 | Sim to | |
| | | | 481,841 | (212.48) | thymopoietin | |
| | | | 83cds(57 | LOC127827 | | |
| | | | 534,300 | | | |
| | | NT_021924? | 88 | | | |
| | 214 | NT_029217− | 89,88,90 | | IL-10, DAF area | |
| | | NT_030585+ | 94,92 | | | |
| | | | 87cds | LOC 127235 | | |
| | | | 578,468 | (214.92) | | |
| | | | 86cds(757- | CR1 | Small fragment | |
| | | | 391,332 | | | |
| | | NT_030579? | Nss | | | |
| | | NT_030575? | Nss | | | |
| | 216 | NT_021877+ | 98cds | LOC126615 | | |
| | | | 1,352.011 | (217) | | |
| | | | 92 | | | |
| | | | 88cds | as above | | |
| | | | 1,411.541 | | | |
| | | NT_030578− | 90cds | KCNH1 | K voltage-gated | Myoblasts at |
| | | | 1,301,974 | (217.84- | channel | fusion stage |
| | | | | 218.23) | | |
| | | | 89cds | KCNH1 | | |
| | | | 1,294.365 | | | |
| | | | 87 | | | |
| | | | 87cds | MGC14801 | | |
| | | | 731,351 | (218.52) | | |
| | | | 86 | | | |
| | | | 90cds | LOC127190 | Sim to p21-Arc | |
| | | | 686,368 | | | |
| | | | 88cds | KCNH1 | | |
| | | | 1,381,704 | | | |
| | | | 88cds | KCNH1 | | |
| | | | 1,265,982 | | | |
| | | | 80cds | LOC127192 | | |
| | | | 1,494,931 | | | |
| | | NT_004993+ | 87cds | LOC128385 | | |
| | | | 694,358 | | | |
| | | | 87,85 | | | |
| | | | 85cds | FLJ10874 | | |
| | | | 378.593 | (219.7) | | |
| | | | 87cds | LOC128391 | | |
| | | | 1,417,141 | | | |
| | | NT_029884? | Nss | | | |
| | 222 | NT_004612− | 93 | | | |
| | | | 87cds | LOC127758 | | |
| | | | 1,675,483 | (221.25) | | |
| | | | 89cds | as above | | |
| | | | 1,674,521 | | | |
| | | | 87cds | LOC127742 | | |
| | | | 577,128 | | | |
| | | | 87cds | LOC127758 | | |
| | | | 1,593,865 | | | |
| | | NT_030582+ | Bits | | | |
| 1q41 | 225 | NT_004817 | 98 | Near | | |
| | | | 773,814 | LOC128150 | | |
| | | | | And | | |
| | | | | LOC128149 | | |
| | | | 96cds | LOC128153 | | |
| | | | 1,098,009 | | | |
| | | | 94 | | | |
| | | | 88cds | FLJ10252 | Glycine rich | RNA |
| | | | 1,364,541 | (225.25) | nucleic acid | processing |
| | | | | | binding domain | protein? |
| | | | 79cds | LOC128155 | | |
| | | | 1,422,107 | | | |
| | | NT_029863+ | Nss | | | |
| | | NT_029871+ | 96cds | RAB3- | GTPase activating | |
| | | | 739,776 | GAP150 | protein | |
| | | | | (228.1) | | |
| | | | 93cds | LOC127083 | | |
| | | | 319,420 | | | |
| | | | 93cds | As above | | |
| | | | 295,189 | | | |
| 1q42 | 229 | NT_004642− | 86 | | | |
| | | | 87cds | LOC127803 | | |
| | | | 146,862 | | | |
| | | | 84cds | LOC127805 | | |
| | | | 306,312 | | | |
| | | NT_029866+ | 86cds | LOC127046 | | |
| | | | 42,308 | | | |
| | | NT_030576? | Nss | | | |
| | 230 | NT_021953+ | 85cds | LOC126708 | | |
| | | | 289,839 | | | |
| | | NT_029858? | Nss | | | |
| | 231 | NT_004861+ | 97 | | | |
| | | | 93cds | LOC128253 | | |
| | | | 348,708 | | | |
| | | | 93 | | | |
| | | | 85cds | FLJ10052 | Contains sushi | |
| | | | 179,795 | (231.6) | domain; sim to | |
| | | | 92 | | DAF precursor | |
| | | NT_004525− | 83,83,93 | | | ADPRTarea |
| | | | 83cds | LOC127586 | Sim to synapsin I | (233.45) |
| | | | 313,463bit | (234.43) | | |
| | 235 | NT_004908+ | 92,87 | LOC128303 | | |
| | | | 85cds | | | |
| | | | 42,367 | | | |
| | | NT_004559+ | Bits | | | |
| | 237 | NT_021973− | bits | | | |
| | 238.5 | NT_004753− | 91cds | DISC1 | Disrupted in | Multiple |
| | | | 543,853 | (238.61- | schizophrenia | sclerosis lesions |
| | | | | 239.13) | | |
| D1S3462 | 238.9 | NT_004753 | In DISC1 | | | |
| | 240 | NT_004433− | 96cds(757 | LOC127452 | | |
| | | | 10,613 | | | |
| | | NT_030561− | 90,88 | | | |
| | | NT_022107? | Nss | | | |
| D1S235 | 242.97 | NT_004836 | In CHS1 | | | |
| | | | 243.07 | | | |
| RYR2 = | 244 | NT_004836− | 99cds | RYR2 | Cardiac | Calcium release |
| 244.3- | | | 2,329,412 | (244.3 | | Channel of |
| 244.9 | | | 98cds | | | sarcoplasmic |
| | | | 2,379197 | LOC128172 | | reticulum |
| D1S235 | | | 92,92 | | | |
| | | | 88cds | | Increased in kidney | |
| | | | 3,235,442 | TM7SF1 | transmembrane | |
| | | | cdspartial | (243.38) | | |
| D1S2785 | 248 | NT_004771+ | 98 | Near | Sim to pentraxin rel | |
| | | | (248.7) | LOC114922 | gene-3 | |
| | | | 1,913,492 | | | |
| | | | 92 | RGS7 | GTPase activating | Mostly in brain |
| | | | 91cds | (147.52) | | |
| | | | 771,848 | | | |
| | 250 | NT_004734+ | 97,87,87 | | | |
| | | | 86cds | AKT3 | v-akt murine | Protein kinase |
| | | | 2,159,228 | (251.3-.4) | thymoma viral | B, gamma; |
| | | | | | oncogene | pleckstrin |
| | | | | | homolog3 | homol domain; |
| | | | 88cds | LOC128041 | | Ser/thr kinase |
| | | | 870,185 | (250.2) | | |
| | | NT_026947? | Nss | | | |
| | | NT_030586? | Bits | | | |
| | 254 | NT_004536+ | 99(252.6) | Near | Sim to olf recs | |
| | | | 340,220 | LOC127615 | | |
| | | | | (309,963) | | |
| | | | | And LOC | | |
| | | | | 127616 | | |
| | | | | (354,490) | | |
| | | | 97 | | | |
| | | | 95cds | FLJ21080 | SET domain; | |
| | | | 1,954,493 | (254.2) | MYND finger | |
| | | | 86 | | Sim to olfrec | |
| | | | 85cds | LOC127610 | | |
| | | | 453,782 | | | |
| | | | 85cds | LOC127622 | | |
| | | | 546,320 | | | |
| | 256.4 | NT_029870+ | 97,92,92 | | Sim to olfactory rec | |
| | | | 94cds | LOC127057 | 1-25 | |
| | | | 21,328 | | | |

TABLE 4
Location of full-length L1 elements on chromosome 16.
Nss signifies no significant homology with nt 1-884
of L1 consensus sequence in accession no. U09116.

| | | | | | L1 element |
|---|---|---|---|---|---|
| | | | | | % identical |
| | | | | | to nt 1-884 |
| M Bases | | | | | of consensus |
| from | | | | | Location |
| ptel | | Marker | Gene | Contig | in contig |
| | | | | NT_010552 | 88 |
| | | | | NT_010540 | Nss |
| | | | | NT_010388 | Nss |
| | | | | NT_027184 | Nss |
| | | | | NT_010543 | Nss |
| | | | | NT_015360 | Nss |
| 6.0 | M | | | NT_027178 | Bit |
| | | | | NT_010384 | Nss |
| | | | | NT_010537 | 84 |
| 11 | M | | | NT_010530 | 98 |
| | | | | | 1,112,885 |
| | | | | | 87 |
| | | | FLJ12668 | | 87cds |
| | | | | | 1,997,396 |
| 12.5 | M | | | NT_010419 | 86 |
| | | | FLJ12363 | | 92cds |
| | | | | | 571,065 |
| | | | | | 86, 83 |
| | | | Myosin | NT_010393 | 86, 86, 89, |
| | | | | | 89 |
| | | | | NT_024760 | Bit |
| 17.3 | M | | LOC115995 | NT_024822 | 99, 3′ utr |
| | | | Sim to | | (208- |
| | | | LIP | | 25,588 |
| | | | isoform | | 91, 87 |
| | | | of BLIP | | |
| | | | | NT_010584 | Nss |
| 20 | M | | | NT_024776 | Nss |
| | | | | NT_030153 | 95 |
| | | | | NT_010436 | Nss |
| | | | | NT_024801 | 88 |
| | | | | NT_010592 | Nss |
| | | | | NT_027182 | 88, 87 |
| | | | | NT_010604 | 89, 88 |
| 27.5 | M | | | NT_010591 | Nss |
| | | | | NT_010441 | 88 |
| | | | | NT_027176 | Nss |
| | | | | NT_010589 | Nss |
| | | | | NT_024802 | Nss |
| 32 | M | | ITGAM | NT_024812 | 88cds |
| | | | (CR3:CD11b) | | 447,353 |
| 44.3 | M | | | NT_028368 | 89 |
| 45 | M | | | NT_024773 | 98 |
| | | | | | 671,001 |
| | | | | | 98 |
| | | | | | 748,545 |
| | | | | | 93, 92, 92, |
| | | | | | 90, 90 |
| | | | | | 88, 87 |
| 45.5 | M | | | NT_010570 | 96, 92, |
| | | | PHKB | | 90cds |
| | | | | | 564,374 |
| | | | | | 89 |
| | | | CDA08 | | 84cds |
| | | | | | 900,968 |
| | | | | | 91 |
| | | | CDA08 | | 91cds |
| | | | | | 1,065,272 |
| | | | CDA08 | | 86cds |
| | | | | | 810,315 |
| | | | | | 88, 85 |
| | | | PHKB | | 82cds |
| | | | | | 700,819 |
| | | | CDA08 | | 87cds |
| | | | | | 822,483 |
| | | | PHKB | | 85cds |
| | | | | | 693,085 |
| | | | | NT_010637 | Nss |
| | | | | NT_010505 | 97, 84, 87 |
| 50 | M | | Near LOC | NT_024779 | 98 |
| | | | 115613 | | 811,937 |
| | | | sim to Na | | 94, 91, 88 |
| | | | and Cl-dep | | 89cds |
| | | | transpo | | 596,749 |
| | | | ZNF267 | | |
| | | | | NT_029461 | Nss |
| | | | | NT_010493 | Nss |
| | | | | NT_010521 | 86, 85, 83 |
| 54 | M | D16S | MGC5149 | NT_010498 | 98cds |
| | | 419 | | | 746,473 |
| | | D16S415 | | | 85, 96, 91 |
| 55.8 | M | | AMFR | NT_019610 | 93, 93 |
| | | | Autocrine | | 89cds |
| | | | motility | | 288,196 |
| | | | factor | | |
| 57.4 | M | | | NT_024766 | bit |
| 58.6 | M | | | NT_010406 | 94, 83 |
| 60.2 | M | | | NT_029457 | 95, 95, 93, |
| | | | | | 86, 86 |
| 62.5 | M | | CDH8x3 | NT_010463 | 97cds |
| | | | Cadherin 8 | CDH8x3 | 1,670,319 |
| | | | | | 96cds |
| | | | | | 1,689,733 |
| | | | | | 95cds |
| | | | | | 1,806583 |
| | | | | | 93, 93, 89 |
| 62.9 | | | | NT_019621 | Nss |
| 63.5 | | | | NT_010615 | 93, 92, 91 |
| 65 (77) | | D16S3253 | 0.1/3.2 | NT_010558 | 95, 87 |
| | | D16S503 | | | |
| 66.6 | | | | NT_010546 | 98 |
| | | | | | 758,900 |
| | | | | | 92, 92 |
| 69.8 | | | Near CDH3 | NT_010478 | 99 |
| | | | (placental) | | 2,329,912 |
| | | | | | 88 |
| 71.1 | | | | NT_019608 | Nss |
| 71.9 | | | | NT_030154 | Nss |
| 72.1 | | | | NT_028369 | bit |
| 72.6 | | | Near DKFZp | NT_010635 | 99 |
| | | | 434L0850 | | 6635 |
| 73.1 | | | | NT_010580 | Nss |
| 73.4 | | | | NT_027175 | 88 |
| 74.2 | | | | NT_024792 | 97, 86 |
| | | | | NT_029456 | Nss |
| 76 | M | | Near | NT_010556 | 99 |
| | | | LOC93220 | | 782,847 |
| | | | Sim to | | |
| | | | laminin | | |
| | | | rec 1 | | |
| | | | | NT_030151 | Nss |
| | | | | NT_024793 | 84 |
| 78 | M | | | NT_010480 | 85 |
| | | | | NT_030152 | Nss |
| | | | | NT_024797 | Bits |
| | | | | NT_026456 | Nss |
| | | | | NT_024827 | 87, 86 |
| | | | | NT_024821 | Nss |
| | | | | NT_010380 | 93 |
| | | | | NT_010422 | 97, 86, 80 |
| | | | | NT_028372 | Nss |
| | | | | NT_010494 | Bits |
| | | | | NT_024814 | 96, 94, 90, |
| | | | | | 84 |
| 86.5 | M | | CDH13 | NT_010428 | 99cds |
| | | | (heart) | | 701,822 |
| | | | | NT_028371 | Nss |
| | | | | NT_024767 | Bit |
| | | | | NT_024788 | 95, 94 |
| | | | | NT_030156 | Nss |
| | | | | NT_019609 | Nss |
| | | | | NT_024772 | Nss |
| | | | | NT_024782 | Nss |
| | | | | NT_024759 | Bit |
| 90.8 | M | | GALNSx2 | NT_010404 | 91cds |
| | | | Galacto- | | 62,543 |
| | | | samine | | 91cds |
| | | | sulfatase | | 69,091 |
| | | | | NT_010632 | Nss |
| 92 | M | | | NT_010542 | Nss |

TABLE 5
Location of full-length L1 elements on chromosome 21.
Nss signifies no significant homology with nt 1-884
of L1 consensus sequence in accession no. U09116

| | | | | L1 element | |
|---|---|---|---|---|---|
| | | | | % identity to | |
| | Location | | | consensus 1-884 | |
| | M Bases | | Contig or | location in | |
| | from ptel | | BAC clone | contig | Gene |
| | 7.8 | M | NT_029490 | 80 | |
| APP | | | NT_011512 | 97 | |
| AD1 | | | | 22,448,995 | |
| | 11-39.6 | M | | 97 | |
| | | | | 1,963,309 | |
| | | | | 97 | |
| | | | | 20,880,812 | |
| | 23.9 | M | | 97cds | APP |
| | | | | 12,869,179 | |
| | | | | 96, 94, 93 | |
| | | | | 93cds | DSCAM |
| | | | | 26,987,787 | |
| | | | | 93, 91 | |
| | | | | 90cds | TTC3 |
| | | | | 24,057,658 | |
| | | | | 89, 89, 88 | |
| | | | | 87cds | DSCAM |
| | | | | 27,064,029 | |
| | | | | 86, 86, 85 | |
| | 11 | M | AP001464 | Nss | |
| | | | AJ239321 | 89 | |
| | | | AP001170 | Nss | |
| | | | AP001135 | Nss | |
| | | | AJ239318 | Nss | |
| | | | AL049911 | Nss | |
| | | | AL050302 | Nss | |
| | | | AL078475 | Nss | |
| | | | AP001465 | Nss | |
| | | | AL163204 | 86 | |
| | | | AL109748 | Nss | |
| | | | AP001466 | Nss | |
| | | | AP001634 | 83 | |
| | | | AP001347 | 89 | |
| | | | AL078615 | Nss | |
| | | | AF130358 | 83 | |
| | | | AF130249 | Nss | STCH |
| | | | AF130247 | Nss | |
| | 12.6 | | AF165138 | Nss | |
| | | | AF198098 | 88 | |
| | | | AF130351 | Nss | |
| | | | AF127936 | 88 | |
| | | | AF248484 | 97 | |
| | | | | 86,680 | |
| | | | | 87 | |
| | | | AF127577 | 97 | |
| | | | | 9772 | |
| | | | | 87 | |
| | | | AF222684 | Nss | |
| | | | AF222685 | Nss | |
| | 13.3 | | AF130248 | Nss | |
| | | | AF246928 | Nss | |
| | | | AJ006998 | Nss | |
| | | | AJ009632 | Nss | |
| | | | AJ006997 | Nss | |
| | | | AL034449 | Nss | |
| | | | AJ010597 | Nss | |
| | | | AJ010598 | 91 | |
| | 14 | | AL109762 | 91 | |
| | | | AP001344 | Nss | |
| | | | AP001346 | 89 | |
| | | | AP001343 | 80 | |
| | | | AP001172 | Nss | |
| | | | AP000962 | Nss | |
| | 14.65 | | AP000473 | Nss | |
| | | | AF130359 | 76 | |
| | | | AF212831 | Nss | |
| | | | AF130418 | 93, 90 | |
| | | | AP001250 | Nss | |
| | | | AP000457 | 85, 93, 85, 86 | |
| | | | AP000968 | 94 | |
| | | | AP000952 | 96 | |
| | | | AP000963 | Nss | |
| | 15.6 | | AP000967 | Nss | |
| | | | AP000432 | Nss | |
| | | | AL109761 | 80 | |
| | | | AP000404 | Nss | |
| | | | AP000745 | Nss | |
| | | | AF165175 | Nss | |
| | 16 | | AP000998 | Nss | |
| | | | AF130417 | Nss | |
| | | | AP000656 | Nss | |
| | | | AL078474 | Nss | |
| | | | AP000456 | Nss | |
| | 16.45 | | AP000455 | Nss | |
| | | | AL109763 | Nss | |
| | | | AF240627 | Nss | |
| | | | AP001538 | 93 | |
| | 17 | | AL157359 | Nss | |
| | | | AP001345 | Nss | |
| | | | AP000431 | Nss | |
| | | | AP000433 | Nss | |
| | | | AP000855 | Nss | |
| | | | AP000958 | Nss | |
| | | | AP000566 | 83 | |
| | | | AP000401 | Nss | |
| | | | AP000403 | Nss | |
| | | | AP000568 | Nss | |
| | | | AP000946 | Nss | |
| | 17.9 | | AF238375 | Nss | |
| | | | AP001256 | Nss | |
| | | | AP001171 | Nss | |
| | 18.3 | | AP001254 | Nss | D21S1437 |
| | | | AP001251 | Nss | |
| | 18.6 | | AP001506 | Nss | |
| | | | AL109772 | Nss | |
| | | | AP000957 | Nss | |
| | | | AP000947 | Nss | |
| | | | AL035532 | Nss | |
| | | | AP000460 | 83 | |
| | 19 | | AF135405 | Nss | |
| | | | AP001138 | Nss | |
| | | | AP001136 | Nss | |
| | | | AP001252 | Nss | |
| | | | AP001137 | Nss | |
| | | | AP001114 | Nss | |
| | | | AP001115 | Nss | |
| | | | AP001117 | Nss | |
| | | | AF241725 | Nss | |
| | | | AP000475 | Nss | |
| | | | AP000472 | Nss | |
| | | | AP000454 | Nss | |
| | 20.05 | | AP000951 | Nss | |
| | | | AP000705 | 82 | |
| | | | AP000657 | Nss | |
| | | | AP000561 | Nss | |
| | | | AP000953 | Nss | |
| | | | AP000959 | 82 | |
| | | | AP000966 | Nss | |
| | | | AP000955 | Nss | |
| | 20.9 | | AP000949 | Nss | |
| | | | AP001116 | Nss | |
| | | | AP001255 | Nss | |
| | | | AP001253 | Nss | |
| | | | AP000459 | Nss | |
| | | | AP000965 | Nss | |
| | | | AP000950 | Nss | |
| | | | AP000961 | Nss | |
| | | | AP000960 | Nss | |
| | | | AP000474 | Nss | |
| | | | AP000477 | Nss | |
| | 22.1 | | AP000470 | Nss | |
| | | | AP000469 | Nss | |
| | | | AP000458 | Nss | |
| | | | AP000954 | Nss | |
| | | | AP000476 | Bit | |
| | | | AP001079 | Nss | |
| | | | AP000964 | Nss | |
| | | | AP000948 | Nss | |
| | | | AP000402 | Nss | |
| | | | AP000146 | Bit | |
| | | | AP001342 | Nss | |
| | | | AP000235 | Nss | |
| | | | AP000234 | Nss | |
| | | | AP000233 | Nss | |
| | | | AP000232 | Nss | |
| | | | AP001341 | Nss | |
| | | | AP001340 | Nss | |
| | | | AP001348 | Nss | |
| | | | AP000220 | Bits | |
| | | | AP000221 | Nss | |
| | | | AL109616 | Nss | |
| | | | AP000223 | Nss | |
| | | | AP000224 | Nss | |
| | | | AP000225 | Nss | |
| | | | AP000226 | Nss | |
| | | | AP000227 | Nss | |
| | | | AP000228 | Nss | |
| | | | AP001443 | Nss | APP |
| | 23.9 | M | AP001442 | 97cds | APP |
| | | | | 7857 | |
| | | | AP001440 | Nss | APP |
| | | | AP001441 | Nss | APP |
| | | | AP001439 | bit | APP |
| | | | AP000229 | Nss | |
| | | | AP000230 | Nss | |
| | | | AP001595 | Nss | |
| | | | AP001596 | Nss | |
| | | | — | | |
| | 33.9 | | AF020802 | Nss | |
| | | | AP000687 | Nss | |
| | | | AP000688 | Nss | |
| | | | AP000689 | Nss | |
| | | | AP000690 | Nss | |
| | | | AP000691 | Nss | |
| | | | AP000692 | 80 | |
| | | | AP000693 | Nss | |
| | | | AP000694 | Nss | |
| | | | AP000695 | Nss | |
| | | | AP000696 | 86 | |
| | 34.6 | | AP000697 | Nss | |
| | | | AP000698 | Nss | |
| | | | AP000699 | Nss | |
| | | | AP000700 | Nss | |
| | | | AP000701 | Nss | |
| | | | AP000702 | Nss | |
| | | | AP000703 | Nss | |
| | | | AP001418 | Nss | |
| | | | AP000704 | Nss | |
| | | | AP001435 | Nss | |
| | | | AP001431 | Nss | |
| | | | AP001429 | 90 | TTC3 |
| | 35.1 | | AP001432 | Nss | TTC3 |
| | | | | | DSCR3 |
| | | | AP001412 | Nss | DSCR3 |
| | | | AP001437 | Nss | |
| | | | AP001428 | Nss | |
| | | | AP001413 | Nss | |
| | | | AP001421 | Nss | |
| | | | AP001414 | Nss | |
| | | | AP001419 | Nss | |
| | | | AP001407 | Nss | |
| | | | AP001436 | Nss | |
| | 35.7 | | AP001416 | Nss | |
| | | | AP001430 | Nss | |
| | | | AP001408 | Nss | |
| | | | AP001411 | Nss | |
| | | | AP001424 | Nss | |
| | | | AP001433 | Nss | |
| | | | AP001427 | Nss | |
| | | | AP001415 | Nss | |
| | | | AP001410 | Nss | |
| | | | AP001420 | Nss | |
| | | | AP001409 | Nss | |
| | | | AP001417 | Nss | |
| | | | AP001425 | Nss | |
| | | | AP001434 | Nss | |
| | 36.4 | | AP001438 | Nss | |
| | | | AP001422 | Nss | |
| | | | AP001423 | Nss | |
| | | | AP001426 | Nss | |
| | | | AP001035 | Nss | |
| | | | AP001036 | Nss | |
| | | | AP001037 | Nss | |
| | | | AP001038 | Nss | |
| | | | AP001039 | Nss | |
| | 36.72 | | AP001040 | Nss | |
| | | | AP001041 | Nss | |
| | | | AP001042 | Nss | |
| | | | AP001043 | Nss | |
| | | | AP001044 | Nss | |
| | | | AP001045 | Nss | |
| | | | AF064858 | Nss | |
| | | | AF064859 | Nss | |
| | | | AF129408 | Nss | |
| | 37.3 | | AF064861 | Nss | |
| | | | AF121781 | Nss | |
| | | | AF121897 | Nss | |
| | 37.6 | | AF064860 | Bit | |
| | | | AF121782 | nss | |
| | | | AF064857 | Nss | |
| | | | AF045449 | Nss | |
| | | | AF064862 | 93, 87, 96 | DSCAM |
| | | | AF064865 | 87 | ″ |
| | | | AF042091 | Nss | ″ |
| | 38.3 | | AF042090 | Nss | ″ |
| | | | AF064863 | Nss | |
| | | | AF165176 | Nss | |
| | | | AF064864 | Nss | |
| | | | AF064866 | Nss | |
| | | | AF043945 | | |
| | | | AL442166 | Nss | |
| | | | AL442167 | Nss | |
| | 39.5 | | AP001610 | nss | |
| | 39.6 | M | — | Nss | |
| | | | NT_030187 | | |
| | 40.5 | | NT_030188 | Bit | |
| | 43 | | NT_011515 | bit | |

What is claimed is:

1. A method of identifying a gene involved in a complex disease comprising the steps of:
(i) identifying a region of the genome neighboring a disease-associated marker;
(ii) comparing the sequence of the 5′ regulatory region of a consensus L1 sequence to the intronic region of genes or predicted genes or to the 5′ or 3′ regulatory region of genes or predicted genes; and
(iii) identifying genes containing a full-length L1 element in their intronic region or containing a full-length L1 element with high sequence fidelity to the L1 consensus sequence in their 5′ or 3′ regulatory region,
wherein said genes identified in step (iii) are involved in a complex disease.

2. The method of claim 1, wherein the high fidelity L1 sequence is at least about 95% similar to the sequence of nucleotides 1-884 of SEQ ID NO: 1.

3. The method of claim 1, wherein the complex disease is selected from the group consisting of systemic lupus erythematosus, multiple sclerosis, insulin-dependent diabetes mellitus, rheumatoid arthritis, phemphigus, psoriasis, autoimmune thyroid disease, scleroderma, mixed connective tissue disease, polymyositis, dermatomyositis, Sjögren's syndrome, pemphigoid, vitiligo, primary biliary cirrhosis, chronic active hepatitis, Crohn's disease, ulcerative colitis, pernicious anemia, schizophrenia, and Alzheimer disease.

4. A method of identifying an individual at risk for or suffering from a complex disease comprising the steps of:
(i) providing a sample from said individual;
(ii) identifying intronic regions of genes containing full-length L1 elements or in 5′ or 3′ regulatory regions of genes containing a full-length high fidelity consensus L1 sequence of the individual's DNA from said sample; and
(iii) comparing said intronic regions of genes or said 5′ or 3′ regulatory regions of step (ii) with a control sample of DNA taken from an individual not susceptible to or at risk for or currently suffering from a complex disease
wherein said genes identified in step (ii) are involved in a complex disease

5. The method of claim 4, wherein the complex disease is selected from the group consisting of systemic lupus erythematosus, multiple sclerosis, insulin-dependent diabetes mellitus, rheumatoid arthritis, phemphigus, psoriasis, autoimmune thyroid disease, scleroderma, mixed connective tissue disease, polymyositis, dermatomyositis, Sjögren's syndrome, pemphigoid, vitiligo, primary biliary cirrhosis, chronic active hepatitis, Crohn's disease, ulcerative colitis, pernicious anemia, schizophrenia, and Alzheimer disease.

6. The method of claim 4, wherein said sample is blood, serum, saliva, urine, tears, sweat, synovial fluid, cerebrospinal fluid, or a solid tissue.

7. A method of identifying an individual at risk for or suffering from a complex disease comprising the steps of:
a. providing a sample from said individual suffering from a complex disease;
b. detecting the amount of L1 DNA, mRNA or a protein encoded by an L1 element in said sample;
c. comparing the amount of step (ii) with an amount of L1DNA, mRNA or a protein obtained from an individual not susceptible to or at risk for or suffering from a complex disease,
wherein if the amount detected in said sample obtained from said individual is greater than the amount of said control, said individual is at risk for or suffering from a complex disease.

8. The method of claim 7, wherein said sample is blood, serum, saliva, urine, tears, sweat, synovial fluid, cerebrospinal fluid, or a solid tissue.

9. The method of claim 7, wherein the complex disease is selected from the group consisting of systemic lupus erythematosus, multiple sclerosis, insulin-dependent diabetes mellitus, rheumatoid arthritis, phemphigus, psoriasis, autoimmune thyroid disease, scleroderma, mixed connective tissue disease, polymyositis, dermatomyositis, Sjögren's syndrome, pemphigoid, vitiligo, primary biliary cirrhosis, chronic active hepatitis, Crohn's disease, ulcerative colitis, pernicious anemia, schizophrenia, and Alzheimer disease.

10. A method of treating or preventing a complex disease, which method comprises administering a therapeutically effective amount of an agent selected from the group consisting of an L1 antisense oligonucleotide, an antibody directed against L1 mRNA, and an antibody directed against a protein encoded by an L1 element.

11. The method of claim 10, wherein the complex disease is selected from the group consisting of systemic lupus erythematosus, multiple sclerosis, insulin-dependent diabetes mellitus, rheumatoid arthritis, phemphigus, psoriasis, autoimmune thyroid disease, scleroderma, mixed connective tissue disease, polymyositis, dermatomyositis, Sjögren's syndrome, pemphigoid, vitiligo, primary biliary cirrhosis, chronic active hepatitis, Crohn's disease, ulcerative colitis, pernicious anemia, schizophrenia, and Alzheimer disease.

12. A method for identifying an individual at risk for or suffering from a complex disease comprising the steps of:
(i) providing a sample obtained from said individual;
(ii) detecting antibodies directed against ribonucleo-protein particles having L1 mRNA complements in said sample
wherein said individual is at risk for or is suffering from a complex disease if said antibodies are present in said sample.

13. The method of claim 12, wherein the complex disease is selected from the group consisting of systemic lupus erythematosus, multiple sclerosis, insulin-dependent diabetes mellitus, rheumatoid arthritis, phemphigus, psoriasis, autoimmune thyroid disease, scleroderma, mixed connective tissue disease, polymyositis, dermatomyositis, Sjögren's syndrome, pemphigoid, vitiligo, primary biliary cirrhosis, chronic active hepatitis, Crohn's disease, ulcerative colitis, pernicious anemia, schizophrenia, and Alzheimer disease.

14. The method of claim 12, wherein said sample is blood, serum, saliva, urine, tears, sweat, synovial fluid, cerebrospinal fluid, or a solid tissue.

15. A method of identifying an individual at risk for or suffering from a complex disease comprising the steps of:
(i) providing a sample obtained from said individual;
(ii) analyzing said sample for the presence of auto antibodies directed against L1 DNA, nRNA or protein products
wherein said individual is at risk for or suffering from a complex disease if said antibodies are present in said sample.

16. The method of claim 15, wherein said sample is blood, serum, saliva, urine, tears, sweat, synovial fluid, cerebrospinal fluid, or a solid tissue.

17. The method of claim 15, wherein the complex disease is selected from the group consisting of systemic lupus erythematosus, multiple sclerosis, insulin-dependent diabetes mellitus, rheumatoid arthritis, phemphigus, psoriasis, autoimmune thyroid disease, scleroderma, mixed connective tissue disease, polymyositis, dermatomyositis, Sjögren's syndrome, pemphigoid, vitiligo, primary biliary cirrhosis, chronic active hepatitis, Crohn's disease, ulcerative colitis, pernicious anemia, schizophrenia, and Alzheimer disease.

18. The method of claim 15, wherein the complex disease is systemic lupus erythematosus.


SEQUENCE LISTING (15 sequences)

SEQ ID NO: 1 - DNA, 6539 bp, Homo sapiens
  ref <308>: DATABASE ACCESSION NUMBER: GenBank Accession No. U09116
  ref <309>: DATABASE ENTRY DATE: 1995-02-02
  ref <313>: RELEVANT RESIDUES: (1)..(6539)
gaataggaacagctccggtctacagctcccagcgtgagcgacgcagaagacggtgatttctgcatttccatctgaggtaccgggttcatctcactagggagtgccagacagtgggcgcaggccagtgtgtgtgcgcaccgtgcgcgagccgaagcagggcgaggcattgcctcacctgggaagcgcaaggggtcagggagttccctttccgagtcaaagaaaggggtgatggacgcacctggaaaatcgggtcactcccacccgaatattgcgcttttcagaccggcttaagaaacggcgcaccacgagactatatcccacacctggctcagagggtcctacgcccacggaatctcgctgattgctagcacagcagtctgagatcaaactgcaaggcggcaacgaggctgggggaggggcgcccgccattgcccaggcttgcttaggtaaacaaagcagccgggaagctcgaactgggtggagcccaccacagctcaaggaggcctgcctgcctctgtaggctccacctctgggggcagggcacagacaaacaaaaaggcagcagtaacctctgcagacttaagtgtccctgtctgacagctttgaagagagcagtggttctcccagcacgcagctggagatctgagaacgggcagactgcctcctcaagtgggtccctgacccctgacccccgagcagcctaactgggaggcaccccccagcaggggcacactgacacctcacacggcagggtattccaacagacctgcagctgagggtcctgtctgttagaaggaaaactaacaaccagaaaggacatctacaccgaaaacccatctgtacatcaccatcatcaaagaccaaaagtagataaaaccacaaagatggggaaaaaacagaacagaaaaactggaaactctaaaacgcagagcgcctctcctcctccaaaggaacgcagttcctcaccagcaacagaacaaagctggatggagaatgattttgacgagctgagagaagaaggcttcagacgatcaaattactctgagctacgggaggacattcaaaccaaaggcaaagaagttgaaaactttgaaaaaaatttagaagaatgtataactagaataaccaatacagagaagtgcttaaaggagctgatggagctgaaaaccaaggctcgagaactacgtgaagaatgcagaagcctcaggagccgatgcgatcaactggaagaaagggtatcagcaatggaagatgaaatgaatgaaatgaagcgagaagggaagtttagagaaaaaagaataaaaagaaatgagcaaagcctccaagaaatatgggactatgtgaaaagaccaaatctacgtctgattggtgtacctgaaagtgatgtggagaatggaaccaagttggaaaacactctgcaggatattatccaggagaacttccccaatctagcaaggcaggccaacgttcagattcaggaaatacagagaacgccacaaagatactcctcgagaagagcaactccaagacacataattgtcagattcaccaaagttgaaatgaaggaaaaaatgttaagggcagccagagagaaaggtcgggttaccctcaaagggaagcctatcagactaacagcagatctctcggcagaaaccctacaagccagaagagagtgggggccaatattcaacattcttaaagaaaagaattttcaacccagaatttcatttccagccaaactaagcttcataagtgaaggagaaagaaaatactttacagacaagcaaatgctgagagattttgtcaccaccaggcctaccctaaaagagctcctgaaggaagcactaaacatggaaaggaacaaccggtaccagccgctgcaaaatcatgccaaaatgtaaagaccatcgagactaggaagaaactgcatcaactaatgagcaaaatcaccagctaacatcataatgacaggatcaaattcacacataacaatattaactttaaatataaatggactaaattctgcaattaaaagacacagactggcaagttggataaagagtcaagacccatcagtgtgctgtattcaggaaacccatctcatgtgcagagacacacataggctcaaaataaaaggatggaggaagatctaccaagcaaatggaaaacaaaaaaaggcaggggttgcaatcctagtctctgataaaacagactttaaaccaacaaagatcaaaagagacaaagaaggccattacataatggtaaagggatcaattcaacaagaggagctaactatcctaaatatttatgcacccaatacaggagcacccagattcataaagcaagtcctgagtgacctacaaagagacttagactcccacacattaataatgggagactttaacaccccactgtcaatattagacagatcaacgagacagaaagtcaacaaggatacccaggaattgaactcagctctgcaccaagcagacctaatagacatctacagaactctccaccccaaatcaacagaatatacatttttttcagcaccacaccacacctattccaaaatcgaccacatagttggaagtaaagctctcctcagcaaatgtaaaagaacagaaattataacaaactatctctcagaccacagtgcaatcaaactagaactcaggattaagaatctcactcaaagccgctcaactacatggaaactgaacaacctgctcctgaatgactactgggtacataacgaaatgaaggcagaaataaagatgttctttgaaaccaacgagaacaaagacaccacataccagaatctctgggacgcattcaaagcagtgtgtagagggaaatttatagcactaaatgcctacaagagaaagcaggaaagatccaaaattgacaccctaacatcacaattaaaagaactagaaaagcaagagcaaacacattcaaaagctagcagaaggcaagaaataactaaaatcagagcagaactgaaggaaatagagacacaaaaaacccttcaaaaaatcaatgaatccaggagctggttttttgaaaggatcaacaaaattgatagaccgctagcaagactaataaagaaaaaaagagagaagaatcaaatagacacaataaaaaatgataaaggggatatcaccaccgatcccacagaaatacaaactaccatcagagaatactacaaacacctctacgcaaataaactagaaaatctagaagaaatggatacattcctcgacacatacactctcccaagactaaaacaggaagaagttgaatctctgaatggaccaataacaggctctgaaattgtggcaataatcaatagtttaccaaccaaaaagagtccaggaccagatggattcacagccgaattctaccagaggtacaaggaggaactggtaccattccttctgaaactattccaatcaatagaaaaagagggaatcctccctaactcattttatgaggccagcatcattctgataccaaagccgggcagagacacaaccaaaaaagagaattttagaccaatatccttgatgaacattgatgcaaaaatcctcaataaaatactggcaaaccgaatccagcagcacatcaaaaagcttatccaccatgatcaagtgggcttcatccctgggatgcaaggctggttcaatatacgcaaatcaataaatgtaatccagcatataaacagagccaaagacaaaaaccacatgattatctcaatagatgcagaaaaagcctttgacaaaattcaacaacccttcatgctaaaaactctcaataaattaggtattgatgggacgtatttcaaaataataagagctatctatgacaaacccacagccaatatcatactgaatgggcaaaaactggaagcattccctttgaaaactggcacaagacagggatgccctctctcaccgctcctattcaacatagtgttggaagttctggccagggcaatcaggcaggagaaggaaataaagggtattcaattaggaaaagaggaagtcaaattgtccctgtttgcagacgacatgattgtttatctagaaaaccccattgtctcagcccaaaatctccttaagctgataagcaacttcagcaaagtctcaggatacaaaatcaatgtacaaaaatcacaagcattcttatacaccaacaacagacaaacagagagccaaatcatgggtgaactcccattcacaattgcttcaaagaggataaaatacctaggaatccaacttacaagggatgtgaaggacctcttcaaggagaactacaaaccactgctcaaggaaataaaagaggacacaaacaaatggaagaacattccatgctcatgggtaggaagaatcaatatcgtgaaaatggccatactgcccaaggtaatttacagattcaatgccatccccatcaagctaccaatgactttcttcacagaattggaaaaaactactttaaagttcatatggaaccaaaaaagagcccgcattgccaagtcaatcctaagccaaaagaacaaagctggaggcatcacactaccttacttcaaactatactacaaggctacagtaaccaaaacagcatggtactggtaccaaaacagagatatagatcaatggaacagaacagagccctcagaaataatgccacatatctacaactatctgatctttgacaaacctgagaaaaacaagcaatggggaaaggattccctatttaataaatggtgctgggaaaactggctagccatatgtagaaagctgaaactggatctcttccttacaccttatacaaaaatcaattcaagatggattaaagatttaaacgttaaacctaaaaccataaaaaccctagaagaaaacctaggcattaccattcaggacataggcgtgggcaaggacttcatgtccaaaacaccaaaagcaatggcaacaaaagacaaaattgacaaatgggatctaattaaactaaagagcttctgcacagcaaaagaaactaccatcagagtgaacaggcaacctacaacatgggagaaaattttcgcaacctactcatctgacaaagggctaatatccagaatctacaatgaactcaaacaaatttacaagaaaaaaacaaacaaccccatcaaaaagtgggcgaaggacatgaacagacacttctcaaaagaagacatttatgcagccaaaaaacacatgaagaaatgctcatcatcactggccatcagagaaatgcaaatcaaaaccactatgagatatcatctcacaccagttagaatggcaatcattaaaaagtcaggaaacaacaggtgctggagaggatgcggagaaataggaacacttttacactgttggtgggactgtaaactagttcaaccattgtggaagtcagtgtggcgattcctcagggatctagaactagaaataccatttgacccagccatcccattactgggtatatacccagaggactataaatcatgctgctataaagacacatgcactcgtatgtttattgcggcactattcacaatagcaaaaacttggaaccaacccaaatgtccaacaatgatagactggattaagaaaatgtggcacatatacaccatggaatattatgcagccataaaaaatgatgagttcatatcctttgtagggacatggatgaaattggaaaccatcattctcagtaaactatcgcaagaacaaaaaaccaaacaccgcatattctcactcataggtgggaattgaacaatgagatcacatggacacaggaaggggaatatcacactctggggactgtggtggggtcgggggaggggggaggggtagcattgggagatatacctaatgctagatgacacattagtgggtgcagcgcaccagcatggcacatgtatacatatgtaactaacctgcacaatgtgcacatgtaccctaaaacttagagtataattaaaaaaaaaaaaaaaaaaaaaaaaaaaaaaaaaaaaagatcacaccactgcactccagcctgggtgtcaaagcgagaccctgtctcaggaaaaaaaaaaaaaaaaaaaaaaaaggcttaattgattgaaccagattcgagaaaacagtgctaaattataattttctcaatactgtaaatatttttcaatcttcagcttcattaacttctataattgaaattatcccaattattacctgacatgtactaaaattccctaaaatggatcttgagtaacattttcacagtacgataatttttctctctgtatatatttatatagtcacatatatgcacatacattatacaagcattacttttctataactgtaaggtcagaatttgaagttgtgttttctttatctttttatttccaatacttggcatcaagttgatattcattagaagtaaaggaggaaggaaatgaataatcttcagatactaagaacattacacttaaattattattaaatctaatttgcattctcatatatggcttagct

SEQ ID NO: 2 - protein, 338 aa, Homo sapiens
  ref <308>: DATABASE ACCESSION NUMBER: GenBank Accession No. U09116
  ref <309>: DATABASE ENTRY DATE: 1995-02-02
  ref <313>: RELEVANT RESIDUES: (1)..(338)
MGKKQNRKTGNSKTQSASPPPKERSSSPATEQSWMENDFDELREEGFRRSNYSELREDIQTKGKEVENFEKNLEECITRITNTEKCLKELMELKTKARELREECRSLRSRCDQLEERVSAMEDEMNEMKREGKFREKRIKRNEQSLQEIWDYVKRPNLRLIGVPESDVENGTKLENTLQDIIQENFPNLARQANVQIQEIQRTPQRYSSRRATPRHIIVRFTKVEMKEKMLRAAREKGRVTLKGKPIRLTADLSAETLQARREWGPIFNILKEKNFQPRISFPAKLSFISEGERKYFTDKQMLRDFVTTRPTLKELLKEALNMERNNRYQPLQNHAKM

SEQ ID NO: 3 - protein, 1275 aa, Homo sapiens [sequence omitted]

SEQ ID NO: 4 - DNA, 830 bp, Homo Sapiens
agttttttcttaaatggtataatatccagtatgaagtattactgaatttgagtaatcattaacaaatatatttcactgccatactgtataccaggctttcttctagggcccagaaatataagctggttaagatccttgattgattgagattacattctaacaggtacagtagacttaatagctaatatcagaaaagattagcagatttattcactgtgttatttgtacttttattctccatttgccttaccctgtatttgaagaaagttttgccttgctttttgatgtgaatgaaattaagcttggatttcacaaccgtggttgaatttaagaaatgttctatttttacatggggaagacggtgctcaagtaatacttgcaggtactagcacccaggatttaggagtccagtccagttttagctacacaaaagtcttaagtacacaaattgccaatagagcagaactatataattcatagatttgctcattattaatctcaaggaaatcagctctttaaatatatgtatttaatgaatgtgaaatttttgggaaggggaactactatgtattaagccataatatttattttacttaaaaaatttttaaacaaagtaatactagtcattgtgagaatgctattctaaaaaaaaaaaaagtcccctggccaccttctctttccatccctagagaccgaacattttcaaaatttgtagctacttcttctacttagcctccatgtattaaactaatatgtgtaataagaataatccgggggaggagccaagatggccgaataggaacagctccggtctacagctcccagcgtga

SEQ ID NO: 5 - DNA, 1103 bp, Homo sapiens
gggaggagccaagatggccgaataggaacagctccggtctacagctcccagcgtgagcgacgcagaagacgggtgatttctgcatttccatctgaggtaccgggttcatctcactagggagtgccagacagtgggcgcaggccagtgtgtgtgcgcaccgtgcgcgagccgaagcagggcgaggcattgcctcacctgggaagcgcaaggggtcagggagttccctttccgagtcaaagaaaggggtgacggacgcacctggaaaatcgggtcactcccacccgaatattgcgcttttcagaccggcttaagaaacggcgcaccacgagactatatcccacacctggctcagagggtcctacgcccacggaatctcgctgattgctagcacagcagtctgagatcaaacggcaaggcggcaacgaggctgggggaggggcgcccgccattgcccaggcttgcttaggcaaacaaagcagctgggaagctcgaactgggtggagcccaccacagctcaaggaggcctgcctgcctctgtaggctccacctctgggggcagggcacagacaaacaaaaagacagcagtaacctctgcagacttaagtgtccctgtctgacagctttgaagagagcagtggttctcccagcacgcagctggagatctgagaacgggcagactgcctcctcaagtgggtccctgacccctgacccccgagcagcctaactgggaggcaccccccagcagggcacactgacacctcacacagcagggtattccaacagacctgcagctgagggtcctgtctgttagaaggaaaactaacaaccagaaaggacatctacaccgaaaacccatctgtacatcaccatcatcaaagaccaaaagtagataaaaccacaaagatggggaaaaaacagaacagaaaaactggaaactctaaaacgcagagcgcctctcctcctccaaaggaacgcagttcctcaccagcaacagaacaaagctggatggagaatgattttgacgagctgagagaagaaggcttcagacgatcaaattactctgagctacgggaggacattcaaaccaaaggcaaagaagttga

SEQ ID NO: 6 - DNA, 1104 bp, Homo sapiens
gggaggagccaagatggccgaataggaacagctccggtctacagctcccagcgtgagcgacgcagaagacgggtgatttctgcatttccatctgaggtaccgggttcatctcactagggagtgccagacagtgggcgcaggccagtgtgtgtgcgcaccgtgcgcgagccgaagcagggcgaggcattgcctcacctgggaagcgcaaggggtcagggagttcccttttcgagtcaaagaaaggggtgacggacgcacctggaaaatcgggtcactcccacccgaatattgcgcttttcagaccggcttaagaaacggcgcaccacgagactatatcccacacctggctcagagggtcctacgcccacggaatctcgctgattgctagcacagcagtctgagatcaaacggcaaggcggcaacgaggctgggggaggggcgcccgccattgcccaggcttgcttaggcaaacaaagcagctgggaagctcgaactgggtggagcccaccacagctcaaggaggcctgcctgcctctgtaggctccacctctgggggcagggcacagacaaacaaaaagacagcagtaacctctgcagacttaagtgtccctgtctgacagctttgaagagagcagtggttctcccagcacgcagctggagatctgagaacgggcagactgcctcctcaagtgggtccctgacccctgacccccgagcagcctaactgggaggcaccccccagcaggggcacactgacacctcacacagcagggtattccaacagacctgcagctgagggtcctgtctgttagaaggaaaactaacaaccagaaaggacatctacaccgaaaacccatctgtacatcaccatcatcaaagaccaaaagtagataaaaccacaaagatggggaaaaaacagaacagaaaaactggaaactctaaaacgcagagcgcctctcctcctccaaaggaacgcagttcctcaccagcaacagaacaaagctggatggagaatgattttgacgagctgagagaagaaggcttcagacgatcaaattactctgagctacgggaggacattcaaaccaaaggcaaagaagttga

SEQ ID NO: 7 - DNA, 600 bp, Homo Sapiens
ctcgctctccacgggttggacccactgcctaaccatttccagtgagatgaactggttacctcagttggagatacagaaatcaccagccttctgagttggtctcgctgggagctgcagaccagagctgttcctatttagccatcttggcccctccccccttgaaaattccatttctttaatagatatagggctattgaggctatttctccttaaatgaacctagatagtttgtgtgcagctgtcaaggaatttgtccattttatctaagttgtcatatttatctatataaagtttttcataatattcgtttattatctatttaccgtctatagcagtactgatggcttttgaatactagcacggctaattgcaaatctatagtcatgtcacctgtctcattcctaagatttaaaaatgcactgcaggacacaaagttattccacacacctcgacttagcttatttgtgtatttcttccaagagaaaaaaaaaaaagaggccaggcatggtggctcacgcctgtaatcccagcactttgggaggctgaggcaggtggatcactttaggtcaggagtttgagatcagcctggccaacatggcg

SEQ ID NO: 8 - DNA, 20 bp, Artificial Sequence
  PCR primer
ctgccatactgtataccagg

SEQ ID NO: 9 - DNA, 20 bp, Artificial Sequence
  PCR primer
ctgttcctattcggccatct

SEQ ID NO: 10 - DNA, 20 bp, Artificial Sequence
  PCR primer
ctagggcccagaaatataag

SEQ ID NO: 11 - DNA, 20 bp, Artificial Sequence
  PCR primer
ccccggattattcttattac

SEQ ID NO: 12 - DNA, 20 bp, Artificial Sequence
  PCR primer
ctggttacctcagttggaga

SEQ ID NO: 13 - DNA, 20 bp, Artificial Sequence
  PCR primer
atgttggccaggctgatctc

SEQ ID NO: 14 - DNA, 20 bp, Artificial Sequence
  PCR primer
agccttctgagttggtctcg

SEQ ID NO: 15 - DNA, 20 bp, Artificial Sequence
  PCR primer
agtgatccacctgcctcagc